(19) United States
(12) Patent Application Publication
(10) Pub. No.: US 2022/0193137 A1
(43) Pub. Date: Jun. 23, 2022

(54) MODIFIED IMMUNE RECEPTOR CONSTRUCTS

(71) Applicants: UMC UTRECHT HOLDING B.V., Utrecht (NL); MILTENYI BIOTEC B.V. & CO. KG, Bergisch Gladbach (DE)

(72) Inventors: Guido Joris Jan Kierkels, Utrecht (NL); Jürgen Herbert Ernst Kuball, Utrecht (NL); Dominik Lock, Bergisch Gladbach (DE); Andrew Didier Mathew Kaiser, Bergisch Gladbach (DE)

(21) Appl. No.: 17/601,384

(22) PCT Filed: Apr. 3, 2020
(86) PCT No.: PCT/EP2020/059606

§ 371 Date: Oct. 4, 2021

(30) Foreign Application Priority Data
Apr. 4, 2019 (EP) 19167423.3

(51) Int. Cl. A61K 35/17; C07K 14/725; C12N 5/0783

(52) U.S. Cl. CPC A61K 35/17; C12N 5/0636; C07K 14/7051

(57) ABSTRACT

The current disclosure provides for immune cells expressing immune receptors comprising i) a polypeptide having an antigen binding domain, a TCR α-chain constant domain, and a TCR δ-chain transmembrane domain; and/or ii) a polypeptide having an antigen binding domain, a TCR β-chain constant domain, and a TCR γ-chain transmembrane domain. The current disclosure also provides for immune cell expressing immune receptors comprising an antigen binding domain, and a transmembrane domain, wherein the antigen binding domain specifically binds a binding moiety that in turn specifically binds a target, for example, a cancer cell. The immune cells, optionally in combination with the binding moiety, can be used in medical treatment, preferably the treatment of a cancer.

### CROSS-REFERENCE TO RELATED APPLICATIONS

This application is a national phase entry under 35 U.S.C. § 371 of International Patent Application PCT/EP2020/059606, filed Apr. 3, 2020, designating the United States of America and published as International Patent Publication WO 2020/201527 A1 on Oct. 8, 2020, which claims the benefit under Article 8 of the Patent Cooperation Treaty to European Union Patent Application Serial No. 19167423.3, filed Apr. 4, 2019.

### TECHNICAL FIELD

The application is in the field of medicine. In particular in the field of gene therapy. It relates to immunology and to cell therapy, particularly for the treatment of cancer. The disclosure further relates to engineered immune cells with modified exogenous immune receptors, and to the use of engineered immune cells in medical treatments.

### STATEMENT ACCORDING TO 37 C.F.R. § 1.821(C) OR (E)—SEQUENCE LISTING SUBMITTED AS A TXT AND PDF FILES

Pursuant to 37 C.F.R. § 1.821(c) or (e), files containing a TXT version and a PDF version of the Sequence Listing have been submitted concomitant with this application, the contents of which are hereby incorporated by reference.

### BACKGROUND

Adoptive transfer of T cells with engineered anti-tumor specificity or anti-pathogen specificity are under development. In such strategies, an exogenous immune receptor such as an alpha beta T cell receptor (TCR), or a gamma delta TCR or a chimeric antigen receptor having a particular anti-tumor specificity, or a particular anti-pathogen specificity is transferred to either autologous T cells from a patient, or, e.g., in case of an allogeneic stem cell transplantation into a patient, in corresponding allogeneic T cells. For example, a leukemic patient that is undergoing blood stem cell transplantation will during the treatment also be lymphodepleted. Hence, such a patient may also benefit from e.g., infusion of donor T cells that have been engineered to express a specific anti-leukemic T cell receptor.

Different engineered TCR concepts have been described previously, for example, in WO2016187349(A1), WO2018026953(A1), WO2018067993(A1), WO2018098365(A2), WO2018119298(A1), and WO2018232020(A1).

Although clinical trials have established the value of adoptive transfer of TCR-engineered cells in cancer patients, clinical benefit of such strategies is generally observed only in some of the patients. One explanation for the observed limited efficacy of TCR-engineered T cells is a suboptimal surface expression of therapeutic TCRs, e.g., caused by competition for CD3 components between the newly introduced and endogenous TCRs. Another limitation of this approach is that the exogenous TCR can recombine with the endogenous TCR naturally expressed in the T cells, forming so called “mixed dimer” formations that can in effect create a new repertoire of T cells potentially capable of reacting with self, and therefore constitute a safety risk for the patient. Moreover, application of such strategies in an allogeneic setting, for example, allogeneic stem cell transplantation, is hampered by serious safety concerns, since non-engineered T cells that express endogenous alpha beta T cell receptors may induce unwanted side effects such as e.g., graft-versus-host disease in an allogeneic stem cell transplantation setting. CARs have another means to restrict T cell targeting to their cognate antigen expressed at the target cell surface (non-MHC dependent) and therefore present advantages over the indicated limitations. “Universal” CAR systems or “adapterCARs” further allow for addressing different antigens using the same CAR. In WO2012082841A2, a universal, yet adaptable, anti-tag chimeric antigen receptor (AT-CAR) system is disclosed that provides T cells with the ability and specificity to recognize and kill target cells, such as tumor cells, that have been marked by tagged antibodies, e.g., FITC- or biotin labeled antibodies. Further concepts of adapterCARs followed e.g., in WO2013044225A1, WO2014100615A1, WO2015057834A1, WO2015058018A1, and WO2016030414A1. However, there is a need for a new and/or improved adapter CAR.

### BRIEF SUMMARY

It was found that the expression of exogenous αβT-cell receptors (αβTCR) on engineered immune cells can be enhanced, while maintaining their functionality, if the α-chain transmembrane domain and/or the β-chain transmembrane domain are replaced by a δ-chain transmembrane domain and/or a γ-chain transmembrane domain respectively, of the human γδTCR counterpart. Also, the optional introduction of an additional Cys bridge was found to lead to enhanced expression and avoid mispairing with endogenous TCR chains.

Accordingly, the disclosure provides for an extracellular and transmembrane frame that not only outcompetes the endogenous (αβ)TCR, but also allows formation of a complex with CD3 that will allow cytoplasmic signaling upon antigen encounter. It was additionally found that the introduction of specific murine derived residues in the amino acid sequence of the receptor may improve expression of the exogenous immune receptor even further. FIGS. 7 and 8 show exemplary embodiments.

A method was also devised for enriching engineered T cells involving the use of a negative selection step. From a mixture of T cells comprising engineered T cells with an exogenous immune receptor and non-engineered T cells with an endogenous alpha beta T cell receptor, the non-engineered alpha beta T cells can be specifically removed from the mixture. The method uses selective antibodies that specifically bind to the endogenous alpha beta T cell receptor, for example, the anti-human αβTCR antibody BW242/412 that is commercially available from Miltenyi (Miltenyi Biotec GmbH, Friedrich-Ebert-Straße 68, 51429 Bergisch Gladbach, Germany).

A modification of only two specific amino acid residues was found in the TCRβ constant domain of the exogenous immune receptor, i.e., T110P and D112G as shown in SEQ ID NO:9 and/or 10 allows for the untouched isolation of αβTCR immune cells with exogenous immune receptors.

The minimal amount of murine residues needed to disrupt binding of anti-human αβTCR was aimed to be mapped, in order to avoid immunogenic effects and a decreased persistence of the engineered immune cells when administered to a patient (see reference 26).

It was found that the T110P and D112G modifications in the TCRβ constant domain of the exogenous immune receptor abrogates binding of an antibody that specifically binds to the human endogenous alpha beta T cell receptor, i.e., the BW242/412 antibody. In addition, it was found that the modification also leads to a further enhanced expression of the exogenous immune receptor, and that the occurrence of mispairing with endogenous immune receptors was decreased.

Accordingly, the BW242/412 antibody can be used to selectively remove immune cells expressing endogenous immune receptors in order to enrich a mixture that comprises immune cells expressing exogenous immune receptors. The BW242/412 antibody can then selectively bind a human endogenous alpha beta T cell receptor, while not substantially binding to an exogenous immune receptor with the specific modification, allowing for enrichment of immune cells expressing exogenous receptors by negative selection.

The preparations of engineered immune cells as obtained with the disclosure are in particular useful in a medical treatment. Such a medical treatment may be the treatment of a cancer. For example, in the treatment of leukemia, a patient undergoing an allogeneic stem cell transplantation may also benefit from an infusion of a preparation of engineered T cells, i.e., allogenic engineered T cells, that are provided by the disclosure, and that are engineered T cells that are provided with enhanced expression of exogenous immune receptors having specificity e.g., for the leukemic cells of the patient. This way, elimination of leukemia may be further promoted in the treatment while the risk of inducing unwanted side effects due to the presence of T cells expressing endogenous alpha beta T cell receptors may be substantially reduced or even avoided altogether.

Similarly, in a different aspect of the disclosure, engineered lymphocytes, i.e., engineered T cells or engineered NK or NKT cells may also be provided with an exogenous immune receptor, e.g., a CAR or an engineered T cell receptor as disclosed herein.

In addition, engineered immune cells with an exogenous immune receptor that can be differentiated from endogenous T cell receptor can be eliminated, i.e., depleted, with a selective antibody via specifically targeting the exogenous immune receptor. The same modification that was used in the enrichment process may be used in the depletion process. A first antibody selectively binds the endogenous alpha beta T cell receptor, while not binding to a modified sequence of the engineered alpha beta T cell receptor in the enrichment method. Conversely, a second antibody now does bind to the modified sequence of the engineered alpha beta T cell receptor but not to the endogenous alpha beta T cell receptor. This way, a minimally modified alpha beta T cell receptor may be provided as an exogenous immune receptor that allows both enrichment and in vivo depletion in combination with two different selective antibodies. All that is required are exogenous immune receptors and selective antibodies that are specific for an endogenous alpha beta T cell receptor, and/or antibodies that are specific for the exogenous immune receptor.

In this regard, it was found that an additional modification of up to seven amino acid residues in the TCRβ constant domain, i.e., in total up to 9 amino acid modifications being Q88H, Y101H, E108K, T110P, Q111E, D112G, R113S, I120N, and V121I as shown in SEQ ID NO:10 allows binding of an anti-murine TCR β antibody, for example, the H57-597 antibody available from BD Pharmingen (BD, 1 Becton Drive, Franklin Lakes, N.J. USA), which then selectively recognizes engineered immune cells with exogenous immune receptors. This can allow for further enhancement, efficacy and reduction of adverse effects caused by non- and poorly-engineered T cells, for example, cytokine release syndrome and/or off-target toxicities. With the additional safety switch, engineered T cells can be depleted at a later time point when needed.

Surprisingly, the up to 9 amino acid modifications render a stronger binding to the anti-murine TCR β antibody than a fully murinized TCRβ constant domain. Without being bound by any theory, it is considered that this differential binding could be a consequence of the fact that the 9/11 sequence contains one less negatively charged residue and therefore results in a more focused electrostatic potential to attract the lysine on CDR1 of anti-MuTCRβ (FIG. 6B).

Finally, the combination of the (humanized) anti-αβTCR antibody and its binding epitope was considered, i.e., the extended mutated region of 9 amino acid modifications in the exogenous TCR β chain constant domain, allows for the use of an adapter concept, wherein an immune cell expressing an immune receptor comprising an antigen binding domain derived from the anti-αβTCR antibody is combined with a polypeptide that can specifically bind a target, for example, a cancer cell, and wherein the polypeptide comprises the extended mutated region of specifically nine amino acids in the exogenous TCR β chain constant domain, such that it can be recognized by the immune cell. See e.g., FIG. 8.

### BRIEF DESCRIPTION OF THE DRAWINGS

FIG. 1. Partial murinization of the TCRβ chain constant domain abrogates binding of the anti-human αβTCR antibody clone BW242/412. (A) Jurma cells were transduced with fully murine (αMuMu/βMuMu), fully human NY-ESO-1 specific (αHuHu/βHuHu) or chimeric αβTCR, in which the α- and β- constant domains were murine, and the variable domains were human NY-ESO-1 specific. Binding of anti-human αβTCR, anti-MuTCRβ and Vβ4 was assessed by flow cytometry. (B) Schematic representation of the constructed αβTCRs that cover all amino acid differences in the TCRα chain and (C) TCRβ chain (upper panels). Jurma cells were transduced with the different murinized αβTCRs after which anti-human αβTCR antibody binding was assessed by flow cytometry (B&C lower panels). Untransduced Jurma cells served as a negative control.

FIG. 2. A combination of two specific murine amino acids in the TM chain constant domain is sufficient to abrogate binding of the anti-human αβTCR antibody clone BW242/412. (A) Jurma cells were transduced with αβTCRs containing single murine amino acid substitutions in the 3^{rd} domain of the β chain after which binding of anti-human αβTCR antibody was assessed using flow cytometry. Untransduced Jurma cells served as a negative control while fully human αβTCR transduced Jurma cells served as a positive control. (B) Jurma cells were transduced with αβTCRs containing combinations of murine amino acids in the 3^{rd} domain of the β chain, after which binding of anti-human αβTCR antibody was assessed using flow cytometry. (C) Visualization of the eleven non-homologous amino acids between human and mouse β chain 3^{rd} domain in cyan using SWISS-MODEL (48) on the modeled template of the β chain of the human JKF6 T-cell receptor (PDB entry code: 4ZDH). Effective single murine amino acid substitutions are displayed in red.

FIG. 3. Primary αβT cells engineered with murinized αβTCRs can be successfully depleted from non- and poorly-engineered immune cells by using anti-human αβTCR antibody clone BW242/412. (A) Primary αβT cells were transduced with minimally murinized αβTCRs with (middle panel) and without (left panel) the “TPDG” mutations. Primary αβT cells with the “TPDG” mutations were MACS-depleted (right-panel). Endogenous αβTCR expression and expression of the introduced αβTCR without the “TPDG” mutations were determined by flow cytometry using anti-human αβTCR antibody. Expression of the introduced βTCR chain was assessed with an anti-Vβ4 antibody (CD4/CD8+) and expression of correctly paired αβTCR chains by NY-ESO-1 pentamers (CD8+) (B) directly after purification and (C) 2 weeks after expansion.

FIG. 4. Efficacy of different strategies to induce preferential pairing of introduced α and βTCR chains. (A) Schematic representation of the three different methods for creating preferential pairing between the introduced α and βTCR chains. TM indicates the transmembrane domain. (B) Primary αβT cells were transduced with the 3 differentially modified αβTCRs as indicated in (A) and expression of the introduced βTCR was determined by an anti-Vβ4 antibody. Pairing of the introduced α and βTCR chains were assessed by NY-ESO-1 pentamers.

FIG. 5. Depletion of non- and poorly-engineered T cells within the context of different preferential αβTCR pairing strategies. Primary αβT cells were transduced with the 3 differently modified αβTCRs as indicated in FIG. 4A and depleted with the anti-human αβTCR antibody clone BW242/412. (A) Directly after depletion, expression of the introduced βTCR was determined by an anti-Vβ4 antibody. (B) Expression of appropriately paired introduced α and βTCR chains were determined by NY-ESO-1 pentamers. (C) Functionality of purified or non-purified engineered immune cells was assessed in a stimulation assay after co-incubation with NY-ESO-1157-165 peptide pulsed T2 cells. IFNγ production was measured in the supernatant by ELISA.

FIG. 6. Depletion of engineered T cells by using a mutation-specific antibody. (A) Jurkat-76 cells were transduced with 5 different murinized αβTCRs to assess binding of anti-MuTCRβ. Wild-type (WT) αβTCR transduced Jurkat-76 cells served as a negative control, while Jurkat-76 transduced with a TCR containing a complete murine constant domain served as a positive control. (B) The structure of the murinized constant domains (βHumm 11/11 and βHumm 9/11) when binding of H57-597 was modeled on the template of the β chain of the murine N15 T-cell receptor (PDB entry code: 1NFD) (49). (C) Primary αβT cells were transduced with 3 different murinized αβTCRs to assess binding of wild-type and chimeric anti-MuTCRβ. anti-Vβ4 and anti-Human IgG1-AF488 isotype were included as positive and negative control respectively. (D) Jurkat-76 were transduced with 4 different murinized αβTCRs and incubated with chimeric H57-MC-VC-PAB-MMAE for 24 hours and then stained with an anti-Vβ4 antibody. (E) Primary αβT cells were transduced with 2 differently murinized αβTCRs, depleted for poorly and non-engineered T cells, expanded using this REP protocol and subsequently incubated with the mutation-specific chimeric antibody H57-MC-VC-PAB-MMAE for 24 hours. Surviving engineered immune cells were determined by NY-ESO-1 pentamer staining.

FIG. 7. TCR design according to an embodiment of the disclosure. At the top, a wild type TCR is compared with a modified TCR according to the disclosure, having a Cyc-Cys bridge, delta and gamma chain transmembrane domains, as well as the 2/11 murinized residues.

FIG. 8. Different chimeric antigen receptor (CAR) designs according to the disclosure. A wild type TCR is compared with a CAR based on a stabilized TCR framework that optionally has the 9/11 murinized residues as described herein. Also shown is an adapter CAR wherein the ScFv(s) can be specific for an antigen capable of working as a linker, for example, a polypeptide including the 9/11 murinized beta-chain constant domain, which can be bound by ScFv's based on the variable domains of an anti-murine Domain 3 antibody, e.g., the H57-597 antibody.

FIG. 9. Graphical representation of different Adapter TCRs.

FIG. 10. 3E5 SupT1 cells were transduced with 10 μl of lentiviral supernatant encoding the different Adapter TCR versions aBioDoc2, aBioDoc3 or aBioDoc11. Three days post transduction, cells were stained with Biotin-PE and analyzed using flow cytometry.

FIG. 11. 2E6 T cells were transduced with an MOI of ˜4 using lentiviral particles encoding the different Adapter TCR versions aBioDoc2, aBioDoc3, aBioDoc11. Cells were stained with Biotin-PE and analyzed using flow cytometry on d5 and d15.

FIG. 12. 5E5 Adapter TCR-expressing T cells (aBioDoc2, aBioDoc3, aBioDoc11) or Mock T-cells and 5E5 GFP+-Rajis or biotinylated GFP+-Rajis (E:T 1:1) were co-cultured with or w/o Rituxifab or Rituximab (1 μg/ml) for 18 h on day 15 and subsequently analyzed using flow cytometry.

FIG. 13. Representative example for assessing intracellular cytokine production. 5E4 Adapter TCR-expressing T cells (aBioDoc11) or Mock T cells and 2.5E5 GFP+-Rajis (E:T 0.25:1) were co-cultured with Rituximab (1 μg/ml) or without Rituximab (neg. ctrl) for 4 h. Subsequently, T cells were stained with CD3-Vioblue, Biotin-PE and IFNg-APC-Vio770 and analyzed using flow cytometry.

FIG. 14A. 5E4 Adapter TCR-expressing T cells (aBioDoc2, aBioDoc3, aBioDoc11) or Mock T-cells and 2.5E5 GFP+-Rajis or biotinylated GFP+-Rajis (E:T 0.25:1) were co-cultured in the absence or presence of either Rituxifab or Rituximab (1 μg/ml, respectively) for 4 h. Subsequently, T cells were stained with CD3-Vioblue, Biotin-PE, TNFa-APC and IFNg-APC-Vio770 and analyzed using flow cytometry.

FIG. 14B. 5E4 Adapter TCR-expressing T cells (aBioDoc2, aBioDoc3, aBioDoc11) or Mock T-cells and 2.5E5 GFP+-Rajis or biotinylated GFP+-Rajis (E:T 0.25:1) were co-cultured in the absence or presence of either Rituxifab or Rituximab (1 μg/ml, respectively) for 4 h. Subsequently, T cells were stained with CD3-Vioblue, Biotin-PE, TNFa-APC and IFNg-APC-Vio770 and analyzed using flow cytometry.

FIG. 15. Alignment of human and murine TCR α and β chains. (A) There is extensive homology between human and murine TCR chains. (B) The differences between the eleven non-homologous amino acids in the 3^{rd} domain of the β chain (βM3).

FIG. 16. Attempting to raise an antibody specific for the T110P+D112G murinized variant of the αβTCR by immunizing 3 Wistar rats with a human-mouse chimeric peptide. (A) Determining the presence of peptide-specific antibodies in the serum of the immunized rats. (B) Assessing the ability of the generated antibodies to bind surface-expressed TCRs. αHumm/βHumm TPDG transduced or non-transduced Jurkat-76 cells were incubated with the indicated percentage of rat serum, after which flow cytometry using anti-RatIgG-FITC was performed. In the controls panel, the functionality of this secondary antibody was confirmed by staining the Jurkat-76 cells with rat anti-HuCD8 followed by anti-RatIgG-FITC. Expression of the TCR was confirmed using anti-Vβ4-FITC. (C) Sequence alignment of the human and murine 3^{rd} domain of the TCRβ chain and the constructed 2/11 and 9/11 murinized variants.

FIG. 17. Chimeric anti-MuTCRβ antibody binds to primary T cells expressing the murinized TCR containing 9 out of 11 murine residues in the 3^{rd} domain of the β chain. Jurkat-76 cells were transduced with 2 different αβTCRs, containing 0/11 or 9/11 murine residues in the 3^{rd} domain of the β chain, to assess binding of the newly generated chimeric and CDR grafted anti-MuTCRβ antibodies. As negative controls, unstained and secondary antibody only conditions were used. As a positive control, wild-type PE-conjugated anti-MuTCRβ was used.

### DETAILED DESCRIPTION

Modified Exogenous Immune Receptor

The disclosure provides for an immune receptor comprising

i) a polypeptide having
an antigen binding domain;
a TCR α-chain constant domain;
a TCR δ-chain transmembrane domain or a TCR γ-chain transmembrane domain;
   and/or

ii) a polypeptide having
an antigen binding domain;
a TCR β-chain constant domain;
a TCR γ-chain transmembrane domain or a TCR δ-chain transmembrane domain.

For example, under i) a TCR δ-chain transmembrane domain may be chosen and under ii) a TCR γ-chain transmembrane domain. Alternatively, under i) a TCR γ-chain transmembrane domain may be chosen and under ii) a TCR δ-chain transmembrane domain.

The immune receptor may be expressed by an immune cell.

It was surprisingly found that an immune receptor design according to the disclosure provides for an enhanced expression of the immune receptor as compared to alternative designs, in particular because it better competes with endogenous TCR, but also because mispairing with endogenous TCR chains can be significantly reduced. Moreover, the instant design can hijack CD3 signaling pathways in the immune cell, which may ultimately lead to a desired therapeutic effect.

In contrast, a natural or endogenous immune receptor (TCR) consists of complete alpha (α) and beta (β) chains, and a minority of immune cells express an alternate receptor, formed by complete gamma (γ) and delta (δ) chains. TCR chains are typically composed of two extracellular domains: a Variable (V) domain and a Constant (C) domain, both of Immunoglobulin superfamily (IgSF) forming antiparallel β-sheets. The Constant domain is proximal to the cell membrane, followed by a transmembrane domain and a short cytoplasmic tail, while the Variable domain can bind an antigen. Accordingly, the Variable domains of both the TCR α-chain and β-chain, or both the TCR γ-chain and δ-chain, each may have three hypervariable or complementarity determining regions (CDRs).

In the immune receptor according to the disclosure
the TCR α-chain constant domain, preferably a human TCR α-chain constant domain, may comprise an amino acid sequence having at least 50, 60, 70, 80, 85, 90, 95, 96, 97, 98, 99, 100% sequence identity with SEQ ID NO:3;
the TCR δ-chain transmembrane domain, preferably a human TCR δ-chain transmembrane domain, may comprise an amino acid sequence having at least 50, 60, 70, 80, 85, 90, 95, 96, 97, 98, 99, 100% sequence identity with SEQ ID NO:4;
the TCR β-chain constant domain, preferably a human TCR β-chain constant domain, may comprise an amino acid sequence having at least 50, 60, 70, 80, 85, 90, 95, 96, 97, 98, 99, 100% sequence identity with SEQ ID NO:5; and/or
the TCR γ-chain transmembrane domain, preferably a human TCR γ-chain transmembrane domain, may comprise an amino acid sequence having at least 50, 60, 70, 80, 85, 90, 95, 96, 97, 98, 99, 100% sequence identity with SEQ ID NO:6.

Additionally or alternatively, the polypeptide under i) and/or the polypeptide under ii) of the immune receptor according to the disclosure may have a cytoplasmic signaling domain, preferably a CD3 signaling domain, more preferably a CD3 gamma, delta, epsilon, or zeta signaling domain, most preferably a CD3 zeta signaling domain. Additionally or as a further alternative, the polypeptide under i) and/or the polypeptide under ii) of the immune receptor according to the disclosure may have a co-stimulatory domain, for example, 4-1BB.

In a preferred variant of the immune receptor according to the disclosure, the polypeptide under i) and the polypeptide under ii) are linked by a Cys bridge, i.e., cysteine bridge, also referred to as S—S bond, or Cys-Cys bridge. It was found that a Cys bridge dramatically stabilizes the design, particularly when the first Cys is located in the TCR α constant domain, preferably located between positions 40-60, more preferably 45-55, or 45-50, most preferably at position 48 as shown in SEQ ID NO:3; and the second Cys is located in the TCR β constant domain, preferably between positions 50-70, more preferably 55-65, 55-60, most preferably at position 57 as shown in SEQ ID NO:5.

The immune receptor may be a T cell receptor or chimeric antigen receptor as described in more detail herein. Alternatively or additionally, the antigen binding domain under i) may be a TCR α-chain variable domain, a receptor or epitope binding molecule/peptide (e.g., a cytokine, a chemokine or a receptor, or a protein binding moiety), preferably an scFv, V-Nar (i.e., (humanized) shark variable domain) or VhH; and/or the antigen binding domain under ii) may be a TCR β-chain variable domain, a receptor or epitope binding molecule/peptide, preferably an scFv, V-Nar or VhH. The antigen binding domain under i) and the antigen binding domain of ii) may be the same or different, i.e., they may bind the same or different antigen.

In a preferred embodiment, the disclosure provides for an (immune cell expressing an) immune receptor according to the disclosure in combination with a tagged polypeptide, for example, together in a (pharmaceutical) composition. Preferably, the antigen binding domain under i) of the immune receptor and/or the antigen binding domain under ii) of the immune receptor is specific for a tag of the tagged polypeptide.

In turn, the tagged polypeptide may be able to specifically bind a target, such as an antigen expressed on a cancer cell. The tag may be a hapten, preferably biotin or FITC or a (recombinant) peptide and/or the tagged polypeptide may comprise an antibody or antigen binding fragment thereof.

Therefore, in a preferred embodiment, the disclosure provides a composition comprising

a) an immune cell expressing an immune receptor comprising
i) a polypeptide having
   an antigen binding domain;
   a TCR α-chain constant domain;
   a TCR δ-chain transmembrane domain or a TCR γ-chain transmembrane domain;
      and/or
ii) a polypeptide having
   an antigen binding domain;
   a TCR β-chain constant domain;
   a TCR γ-chain transmembrane domain or a TCR δ-chain transmembrane domain,
wherein the antigen binding domain under i) and/or ii) specifically binds a tag of a tagged polypeptide, wherein preferably the polypeptide binds specifically to an antigen expressed on the surface of a target cell, and

b) the tagged polypeptide.

For example, under i) a TCR δ-chain transmembrane domain may be chosen and under ii) a TCR γ-chain transmembrane domain. Alternatively, under i) a TCR γ-chain transmembrane domain may be chosen and under ii) a TCR δ-chain transmembrane domain.

The composition, wherein in the immune cell expressing the immune receptor the polypeptide under i) and the polypeptide under ii) are preferably linked by a Cysteine bridge.

The antigen binding domain under i) may be an epitope binding peptide, preferably an scFv, V-Nar, or VhH; and/or the antigen binding domain under ii) may be an epitope binding peptide, preferably an scFv, V-Nar or VhH,
wherein preferably the antigen binding domain under i) and the antigen binding domain of ii) may be the same or different.

The tag of the tagged polypeptide may be a hapten such a biotin that may be coupled to the polypeptide covalently (a biotinylated polypeptide). In this embodiment of the disclosure, the antigen binding domain under i) or ii) may comprise an amino acid sequence, e.g., a svFc, that specifically may bind to biotin. The antigen binding domain may be derived from an anti-biotin antibody.

In a particularly preferred embodiment, the (tagged) polypeptide and/or the tag comprises a murine derived epitope amino acid sequence.

The murine derived amino acid sequence as referred to herein may be an amino acid sequence having at least 50, 60, 70, 80, 85, 90, 95, 96, 97, 98, 99, 100% sequence identity with SEQ ID NO:35 and/or SEQ ID NO:36, and/or may be characterized in that the amino acid sequence comprises:
an amino acid other than Threonine at a position corresponding to position 110 as shown in SEQ ID NO:9 and/or SEQ ID NO:10; and
an amino acid other than Aspartic acid at a position corresponding to position 112 as shown in SEQ ID NO:9 and/or SEQ ID NO:10. Preferably, but not essential, the amino acid sequence is not an amino acid sequence according to SEQ ID NO:8.

The amino acid other than Threonine at a position corresponding to position 110 preferably is Proline or conservative substitution thereof. The amino acid other than Aspartic acid at a position corresponding to position 112 preferably is Glycine or conservative substitution thereof.

Preferably, the murine derived amino acid sequence as referred herein is an amino acid sequence having at least 50, 60, 70, 80, 85, 90, 95, 96, 97, 98, 99, 100% sequence identity with SEQ ID NO:35 and/or SEQ ID NO:36 that, in addition to the amino acids at positions 110 and 112, further comprises:
an Histidine or conservative substitution thereof at a position corresponding to position 88 as shown in SEQ ID NO:10;
an Histidine or conservative substitution thereof at a position corresponding to position 101 as shown in SEQ ID NO:10.
a Lysine or conservative substitution thereof at a position corresponding to position 108 as shown in SEQ ID NO:10;
a Glutamic acid or conservative substitution thereof at a position corresponding to position 111 as shown in SEQ ID NO:10;
a Serine or conservative substitution thereof at a position corresponding to position 113 as shown in SEQ ID NO:10;
an Asparagine or conservative substitution thereof at a position corresponding to position 120 as shown in SEQ ID NO:10; and/or
an Isoleucine or conservative substitution thereof at a position corresponding to position 121 as shown in SEQ ID NO:10.

The disclosure further provides that the (murine derived epitope) amino acid sequence is an amino acid sequence that (additionally) has:
an Asparagine or conservative substitution thereof at a position corresponding to position 106 as shown in SEQ ID NO:9 and/or SEQ ID NO:10 (or position 19 as shown in SEQ ID NO:7); and/or
an Alanine or conservative substitution thereof at a position corresponding to position 114 as shown in SEQ ID NO:9 and/or SEQ ID NO:10 (or position 27 in SEQ ID NO:7).

To be able to bind the above (murine derived epitope) amino acid sequence, the antigen binding domain under i) and/or the antigen binding domain under ii) of the immune receptor according to the disclosure preferably comprises:
an amino acid sequence having at least 50, 60, 70, 80, 85, 90, 95, 96, 97, 98, 99, 100% sequence identity with SEQ ID NO:1; and/or
an amino acid sequence having at least 50, 60, 70, 80, 85, 90, 95, 96, 97, 98, 99, 100% sequence identity with SEQ ID NO:2.

Alternatively, the (immune cell expressing the) immune receptor according to the disclosure may itself comprise the murine derived epitope amino acid sequence as disclosed herein. The murine derived epitope amino acid sequence may be comprised in a TCR Cβ domain, preferably domain 3 thereof.

Adapter Concept

The disclosure also provides for an immune receptor comprising
an antigen binding domain comprising an amino acid sequence having at least 50, 60, 70, 80, 85, 90, 95, 96, 97, 98, 99, 100% sequence identity with SEQ ID NO:1; and/or an amino acid sequence having at least 50, 60, 70, 80, 85, 90, 95, 96, 97, 98, 99, 100% sequence identity with SEQ ID NO:2;
a transmembrane domain,
   wherein the immune cell preferably is in combination with a polypeptide that comprises the murine derived epitope amino acid sequence as disclosed herein.

The adapter concept provides for new and advantageous applications. Specifically, the antigen binding domain may be specific for the polypeptide, wherein preferably the polypeptide can specifically bind a target. Alternatively, the immune receptor has a different antigen binding domain that is capable of binding a (different) antigen that can act as a linker, wherein, for example, the linker (e.g., a polypeptide) can specifically bind to a cancer cell epitope.

The immune receptor may be expressed by an immune cell and may be a T cell receptor or chimeric antigen receptor (CAR) as described in more detail herein. The immune cell may be a human immune cell, preferably a human T cell or human NK cell.

Immune cell(s) expressing the immune receptor can be used in a medical treatment, preferably for use in the treatment of a cancer. The immune cells can be administered separately, sequentially or simultaneously to administration of the polypeptide.

In a preferred embodiment, the antigen binding domain is a (human) antigen binding domain, preferably an scFv and/or the transmembrane domain is (human) transmembrane domain, preferable a CD8 or CD8α transmembrane domain.

In a particularly preferred embodiment, the immune receptor according to the disclosure comprises an amino acid sequence that has at least 50, 60, 70, 80, 85, 90, 95, 96, 97, 98, 99, 100% sequence identity with SEQ ID NO:11.

Additionally or alternatively, the immune receptor has a cytoplasmic signaling domain, preferably a CD3 zeta cytoplasmic signaling domain and/or a 4-1BB cytoplasmic signaling domain.

Below, exemplary embodiments of the adapter concept as disclosed herein are described.

1. Immune cell expressing an immune receptor (CAR) comprising
an antigen binding domain comprising an amino acid sequence having at least 70, 80, 90, 95, 99 or 100% sequence identity with SEQ ID NO:1; and/or an amino acid sequence having at least 70, 80, 90, 95, 99 or 100% sequence identity with SEQ ID NO:2;
optionally a transmembrane domain,
   wherein the antigen binding domain can specifically bind or recognize a polypeptide, preferably wherein the polypeptide is bound to a different (soluble) immune receptor; the polypeptide comprising an amino acid sequence having at least 70, 80, 90, 95, 99 or 100% sequence identity with SEQ ID NO:36, characterized in that the amino acid sequence comprises:
an Histidine or conservative substitution thereof at a position corresponding to position 88 as shown in SEQ ID NO:10;
an Histidine or conservative substitution thereof at a position corresponding to position 101 as shown in SEQ ID NO:10.
a Lysine or conservative substitution thereof at a position corresponding to position 108 as shown in SEQ ID NO:10;
an amino acid other than Threonine (preferably Proline) at a position corresponding to position 110 as shown in SEQ ID NO:9 and/or SEQ ID NO:10;
a Glutamic acid or conservative substitution thereof at a position corresponding to position 111 as shown in SEQ ID NO:10;
an amino acid other than Aspartic acid (preferably Glycine) at a position corresponding to position 112 as shown in SEQ ID NO:9 and/or SEQ ID NO:10.
a Serine or conservative substitution thereof at a position corresponding to position 113 as shown in SEQ ID NO:10;
an Asparagine or conservative substitution thereof at a position corresponding to position 120 as shown in SEQ ID NO:10; and/or
an Isoleucine or conservative substitution thereof at a position corresponding to position 121 as shown in SEQ ID NO:10.

The different (soluble) immune receptor that is bound to the polypeptide may be e.g., an antibody or antigen binding fragment thereof, or any other peptide that may bind specifically to an antigen expressed on a target cell.

The immune cell may be in combination with the polypeptide, for example, in a (pharmaceutical) composition.

2. Immune cell expressing an immune receptor according to embodiment 1, wherein
the amino acid other than Threonine at a position corresponding to position 110 is Proline or conservative substitution thereof; and/or
the amino acid other than Aspartic acid at a position corresponding to position 112 is Glycine or conservative substitution thereof.

3. Immune cell expressing an immune receptor according to any one of the previous embodiments, wherein the amino acid sequence having at least 70% sequence identity with SEQ ID NO:36 further comprises:
an Asparagine or conservative substitution at a position corresponding to position 106 as shown in SEQ ID NO:10; and/or
an Alanine or conservative substitution at a position corresponding to position 114 as shown in SEQ ID NO:10.

4. Immune cell expression an immune receptor according to any one of the previous embodiments, wherein the amino acid sequence having at least 70, 80, 80, 95, 99, or 100% sequence identity with SEQ ID NO:36 is not an amino acid sequence according to SEQ ID NO:8.

5. Immune cell expressing an immune receptor according to any one of the previous embodiments, wherein the antigen binding domain is specific for the polypeptide, wherein preferably the polypeptide can specifically bind a target.

6. Immune cell expressing an immune receptor according to any one of the previous embodiments, wherein the immune receptor is a chimeric antigen receptor.

7. Immune cell expressing an immune receptor according to any one of the previous embodiments,

wherein
the antigen binding domain is an scFv and/or
the transmembrane domain is a CD8 or CD8α transmembrane domain.

8. Immune cell expressing an immune receptor according to any one of the previous embodiments, wherein the immune receptor comprises an amino acid sequence that has at least 80%, 90%, 100% sequence identity with SEQ ID NO:11.

9. Immune cell expressing an immune receptor according to any one of the previous embodiments, wherein the immune receptor further has a cytoplasmic signaling domain, preferably a CD3 zeta cytoplasmic signaling domain and/or a 4-1BB cytoplasmic signaling domain.

10. Immune cell expressing an immune receptor according to any one of the previous embodiments, wherein the immune cell is human immune cell, preferably a human T cell or human NK cell or human NK T cells.

11. Immune cell(s) expressing exogenous immune receptors according to any one of the previous embodiments for use in a medical treatment, preferably for use in the treatment of a cancer and/or wherein the immune cells are administered separately, sequentially or simultaneously to the polypeptide according to any one of the previous embodiments.

In another aspect, provided is a composition comprising

a) an immune cell expressing a CAR comprising
i) an antigen binding domain specific for a tag of a tagged polypeptide
ii) a transmembrane domain
iii) an intracellular signaling domain
wherein the antigen binding domain specifically binds a tag of a tagged polypeptide, wherein the polypeptide binds specifically to an antigen expressed on the surface of a target cell, wherein the target cell is a cancer cell, and

b) the tagged polypeptide,
wherein the tag comprises an amino acid sequence having at least 70, 80, 90, 99, or 100% sequence identity with SEQ ID NO:36, characterized in that the amino acid sequence comprises:
   an Histidine or conservative substitution thereof at a position corresponding to position 88 as shown in SEQ ID NO:10;
   an Histidine or conservative substitution thereof at a position corresponding to position 101 as shown in SEQ ID NO:10.
   a Lysine or conservative substitution thereof at a position corresponding to position 108 as shown in SEQ ID NO:10;
   an amino acid other than Threonine (preferably Proline) at a position corresponding to position 110 as shown in SEQ ID NO:9 and/or SEQ ID NO:10;
   a Glutamic acid or conservative substitution thereof at a position corresponding to position 111 as shown in SEQ ID NO:10;
   an amino acid other than Aspartic acid (preferably Glycine) at a position corresponding to position 112 as shown in SEQ ID NO:9 and/or SEQ ID NO:10.
   a Serine or conservative substitution thereof at a position corresponding to position 113 as shown in SEQ ID NO:10;
   an Asparagine or conservative substitution thereof at a position corresponding to position 120 as shown in SEQ ID NO:10; and/or
   an Isoleucine or conservative substitution thereof at a position corresponding to position 121 as shown in SEQ ID NO:10.

The antigen binding domain specific for the tag may comprise an amino acid sequence having at least 70, 80, 90, or 100% sequence identity with SEQ ID NO:1; and/or an amino acid sequence having at least 70, 80, 90, or 100% sequence identity with SEQ ID NO:2.

The immune cell may be a T cell or a NK cell.

The CAR, wherein the intracellular signaling domain comprises at least a primary signaling domain such as CD3zeta and at least a co-stimulatory signaling domain such as CD137 or CD28.

The tagged polypeptide that binds to an antigen expressed on the surface of a cell may be an antibody or antigen binding fragment thereof.

In another aspect, provided is a pharmaceutical composition comprising

a) an immune cell expressing a CAR comprising
i) an antigen binding domain specific for a tag of a tagged polypeptide
ii) a transmembrane domain
iii) an intracellular signaling domain
wherein the antigen binding domain specifically binds a tag of a tagged polypeptide, wherein the polypeptide binds specifically to an antigen expressed on the surface of a target cell, wherein the target cell is a cancer cell, and

b) the tagged polypeptide,
wherein the tag comprises an amino acid sequence having at least 70, 80, 90, 95, 99 or 100% sequence identity with SEQ ID NO:36, characterized in that the amino acid sequence comprises:
   an Histidine or conservative substitution thereof at a position corresponding to position 88 as shown in SEQ ID NO:10;
   an Histidine or conservative substitution thereof at a position corresponding to position 101 as shown in SEQ ID NO:10.
   a Lysine or conservative substitution thereof at a position corresponding to position 108 as shown in SEQ ID NO:10;
   an amino acid other than Threonine (preferably Proline) at a position corresponding to position 110 as shown in SEQ ID NO:9 and/or SEQ ID NO:10;
   a Glutamic acid or conservative substitution thereof at a position corresponding to position 111 as shown in SEQ ID NO:10;
   an amino acid other than Aspartic acid (preferably Glycine) at a position corresponding to position 112 as shown in SEQ ID NO:9 and/or SEQ ID NO:10.
   a Serine or conservative substitution thereof at a position corresponding to position 113 as shown in SEQ ID NO:10;
   an Asparagine or conservative substitution thereof at a position corresponding to position 120 as shown in SEQ ID NO:10; and/or
   an Isoleucine or conservative substitution thereof at a position corresponding to position 121 as shown in SEQ ID NO:10.

The antigen binding domain specific for the tag may comprise an amino acid sequence having at least 70, 80, 90, 95, 99 or 100% sequence identity with SEQ ID NO:1; and/or an amino acid sequence having at least 70, 80, 90, 95, 99 or 100% sequence identity with SEQ ID NO:2.

The pharmaceutical composition optionally may comprise a pharmaceutical acceptable carrier together with the immune cells and/or together with the tagged polypeptide.

In another aspect, provided is a composition for the use in the treatment of a subject suffering from cancer, wherein the cancer cells express an antigen, the composition comprising

a) an immune cell expressing a CAR comprising
i) an antigen binding domain specific for a tag of a tagged polypeptide
ii) a transmembrane domain
iii) an intracellular signaling domain
wherein the antigen binding domain specifically binds a tag of a tagged polypeptide, wherein the polypeptide binds specifically to the antigen expressed on the surface of a target cell, wherein the target cell is a cancer cell, and

b) the tagged polypeptide,
wherein the tag comprises an amino acid sequence having at least 70, 80, 90, 95, 99 or 100% sequence identity with SEQ ID NO:36, characterized in that the amino acid sequence comprises:
   an Histidine or conservative substitution thereof at a position corresponding to position 88 as shown in SEQ ID NO:10;
   an Histidine or conservative substitution thereof at a position corresponding to position 101 as shown in SEQ ID NO:10.
   a Lysine or conservative substitution thereof at a position corresponding to position 108 as shown in SEQ ID NO:10;
   an amino acid other than Threonine (preferably Proline) at a position corresponding to position 110 as shown in SEQ ID NO:9 and/or SEQ ID NO:10;
   a Glutamic acid or conservative substitution thereof at a position corresponding to position 111 as shown in SEQ ID NO:10;
   an amino acid other than Aspartic acid (preferably Glycine) at a position corresponding to position 112 as shown in SEQ ID NO:9 and/or SEQ ID NO:10.
   a Serine or conservative substitution thereof at a position corresponding to position 113 as shown in SEQ ID NO:10;
   an Asparagine or conservative substitution thereof at a position corresponding to position 120 as shown in SEQ ID NO:10; and/or
   an Isoleucine or conservative substitution thereof at a position corresponding to position 121 as shown in SEQ ID NO:10.

The antigen binding domain specific for the tag may comprise an amino acid sequence having at least 70, 80, 90, 95, 99, 100% sequence identity with SEQ ID NO:1; and/or an amino acid sequence having at least 70, 80, 90, 95, 100% sequence identity with SEQ ID NO:2.

Another aspect of the disclosure provides a method for treatment of a subject suffering from cancer, wherein the cancer cells express an antigen, the method comprising the step of applying a CAR as disclosed herein comprising at least one antigen binding domain specific for the antigen to the patient, or of applying a composition as disclosed herein comprising

a) an immune cell expressing a CAR comprising
i) an antigen binding domain specific for a tag of a tagged polypeptide
ii) a transmembrane domain
iii) an intracellular signaling domain
wherein the antigen binding domain specifically binds a tag of a tagged polypeptide, wherein the polypeptide binds specifically to the antigen expressed on the surface of a target cell, wherein the target cell is a cancer cell, and

b) the tagged polypeptide,
wherein the tag comprises an amino acid sequence having at least 70, 80, 90, 95, 99 or 100% sequence identity with SEQ ID NO:36, characterized in that the amino acid sequence comprises:
   an Histidine or conservative substitution thereof at a position corresponding to position 88 as shown in SEQ ID NO:10;
   an Histidine or conservative substitution thereof at a position corresponding to position 101 as shown in SEQ ID NO:10.
   a Lysine or conservative substitution thereof at a position corresponding to position 108 as shown in SEQ ID NO:10;
   an amino acid other than Threonine (preferably Proline) at a position corresponding to position 110 as shown in SEQ ID NO:9 and/or SEQ ID NO:10;
   a Glutamic acid or conservative substitution thereof at a position corresponding to position 111 as shown in SEQ ID NO:10;
   an amino acid other than Aspartic acid (preferably Glycine) at a position corresponding to position 112 as shown in SEQ ID NO:9 and/or SEQ ID NO:10.
   a Serine or conservative substitution thereof at a position corresponding to position 113 as shown in SEQ ID NO:10;
   an Asparagine or conservative substitution thereof at a position corresponding to position 120 as shown in SEQ ID NO:10; and/or
   an Isoleucine or conservative substitution thereof at a position corresponding to position 121 as shown in SEQ ID NO:10.

The antigen binding domain specific for the tag may comprise an amino acid sequence having at least 70, 80, 90, 95, 99 or 100% sequence identity with SEQ ID NO:1; and/or an amino acid sequence having at least 70, 80, 90, 95, 99 or 100% sequence identity with SEQ ID NO:2.

In another embodiment, the CAR system (composition comprising the CAR specific for a tag of a tagged polypeptide (“anti-tag CAR”) and the polypeptide specifically binding to a target antigen as disclosed herein) may be for use in the treatment in a subject suffering from cancer. Cells such as immune cells, e.g., T cells or NK cells of a subject, may be isolated or established immune cell lines may be used. The subject may suffer from the cancer (a patient) or may be a healthy subject. These immune cells are genetically modified in vitro to express the CAR specific for a tag of a tagged polypeptide as disclosed herein. These engineered cells may be activated and expanded in vitro to a therapeutically effective population of expressing cells. In cellular therapy these engineered cells may be infused to a recipient in need thereof as a pharmaceutical composition (or a formulation of a therapeutically effective population of anti-tag CAR expressing cells), in addition to a second pharmaceutical composition, a formulation of the tagged polypeptide as disclosed herein. The infused cells in the recipient may be able to kill (or at least stop growth of) cancerous cells expressing the antigen that is recognized by the CAR system as disclosed herein. The recipient may be the same subject from which the cells were obtained (autologous cell therapy) or may be from another subject of the same species (allogeneic cell therapy).

The therapeutically effective population of anti-tag CAR expressing cells may be administered to the patient before the administration of the formulation of the tagged polypeptide to the subject. Alternatively, the formulation of the tagged polypeptide may be administered to the subject before or at the same time as the administration the therapeutically effective population of anti-tag CAR expressing cells to the subject.

Populations of anti-tag-CAR-expressing (immune) cells may be formulated for administered to a subject using techniques known to the skilled artisan.

Formulations comprising therapeutically effective population(s) of anti-tag expressing CAR cells may include pharmaceutically acceptable excipient(s) (carrier or diluents). Excipients included in the formulations will have different purposes depending, for example, on the nature of the tag-binding domain of the anti-tag-CAR, the (sub)population of immune cells used, and the mode of administration. Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents.

A formulation of a therapeutically effective population(s) of anti-tag expressing CAR cells may include one population of anti-tag CAR-expressing (immune) cells, or more than one population of anti-tag-CAR-expressing (immune) cells. The different populations of anti-tag-CAR (immune) cells may vary based on the identity of the tag-binding domain, the identity of the activation domain, the identity of the (sub)population of immune cells, or a combination thereof.

The formulations comprising therapeutically effective population(s) of anti-tag expressing CAR cells may be administered to a subject using modes and techniques known to the skilled artisan. Exemplary modes include, but are not limited to, intravenous injection. Other modes include, without limitation, intratumoral, intradermal, subcutaneous (s.c, s.q., sub-Q, Hypo), intramuscular (i.m.), intraperitoneal (i.p.), intra-arterial, intramedulary, intracardiac, intra-articular (joint), intrasynovial (joint fluid area), intracranial, intraspinal, and intrathecal (spinal fluids).

The formulations comprising therapeutically effective population(s) of anti-tag expressing CAR cells that are administered to a subject comprise a number of anti-tag-CAR-expressing cells such immune cells that is effective for the treatment of the specific indication or disorder.

In general, formulations may be administered that comprise between about 1×10⁴ and about 1×10¹⁰ anti-tag-CAR-expressing cells such as immune cells. In most cases, the formulation may comprise between about 1×10⁵ and about 1×10⁹ anti-tag-CAR-expressing cells such as immune cells, from about 5×10⁵ to about 5×10⁸ anti-tag-CAR-expressing cells such as immune cells, or from about 1×10⁶ to about 1×10⁷ anti-tag-CAR-expressing cells such as immune cells. However, the number of anti-tag-CAR-expressing cells such as immune cells administered to a subject may vary between wide limits, depending upon the location, source, identity, extent and severity of the disorder, the age and condition of the individual to be treated, etc. A physician may ultimately determine appropriate dosages to be used.

The tagged polypeptides as disclosed herein may be formulated for administered to a subject using techniques known to the skilled artisan. Formulations of the tagged polypeptides may include pharmaceutically acceptable excipient(s) (carriers or diluents). Excipients included in the formulations will have different purposes depending, for example, on the nature of the tag, the antigen binding domain of the tagged polypeptide, and the mode of administration. Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents.

A formulation of tagged polypeptide may include one type of tag polypeptide, or more than one type of tagged polypeptides. The different types of tagged polypeptides may vary based on the identity of the tag, the antigen binding moiety of the tagged polypeptide, or a combination thereof.

The tagged polypeptides may be administered to a subject using modes and techniques known to the skilled artisan. Exemplary modes include, but are not limited to, intravenous, intraperitoneal, and intratumoral injection. Other modes include, without limitation, intradermal, subcutaneous (s.c, s.q., sub-Q, Hypo), intramuscular (i.m.), intra-arterial, intramedulary, intracardiac, intra-articular (joint), intrasynovial (joint fluid area), intracranial, intraspinal, and intrathecal (spinal fluids).

Formulations comprising the polypeptide are administered to a subject in an amount that is effective for treating the specific indication or disorder. In general, formulations comprising at least about 1 μg/kg to about 100 mg/kg body weight of the tagged polypeptide may be administered to a subject in need of treatment. In most cases, the dosage may be from about 100 μg/kg to about 10 mg/kg body weight of the tagged polypeptide daily, taking into account the routes of administration, symptoms, etc. The amount of tagged polypeptide in formulations administered to a subject may vary between wide limits, depending upon the location, source, identity, extent and severity of the disorder, the age and condition of the individual to be treated, etc. A physician may ultimately determine appropriate dosages to be used.

The timing between the administration of the CAR expressing cell formulation and the tag polypeptide-formulation may range widely depending on factors that include the type of (immune) cells being used, the binding specificity of the CAR, the identity of the tag, the antigen binding moiety of the tagged polypeptide, the identity of the target cell, e.g., cancer cell to be treated, the location of the target cell in the subject, the means used to administer the formulations to the subject, and the health, age and weight of the subject being treated. Indeed, the tagged polypeptide formulation may be administered prior to, simultaneous with, or after the genetically engineered (immune) cell formulation.

Depending on the disorder being treatment the step of administering the CAR expressing cell formulation, or the step of administering the tagged polypeptide formulation, or both, can be repeated one or more times. When two or more formulations of tagged polypeptides may be applied to a subject, the formulations applied may comprise the same or different tagged polypeptides. When two or more formulations of engineered cells such as immune cells expressing the CAR of the disclosure are applied to a subject, the engineered cells may be of the same cell type or of different cell types, e.g., T cells and/or NK cells. A formulation of cells such as immune cells may also comprise more than one cell type, each expressing the CAR of the disclosure.

### Further Embodiments

The disclosure also provides for a nucleic acid encoding any of the (exogenous) immune receptors as disclosed herein. The nucleic acid may be comprised in a vector and/or comprised in a host cell that may or may not be an immune cell.

The immune cells according to the disclosure may be immune cells that are engineered to express an exogenous immune receptor. The immune cell according to the disclosure may be a human immune cell, preferably a human T cell or human NK cell. The exogenous immune receptor may have the same function as an endogenous T cell receptor with regard to antigen recognition and T cell action. Non-engineered immune cells are cells that express an endogenous immune receptor, i.e., T cell receptor. Endogenous T cell receptors are either of the γδ T cell receptor type or αβ T cell receptor type.

An exogenous immune receptor according to the disclosure is preferably defined as not being an endogenous T cell receptor. For example, an exogenous immune receptor may be a particular selected γδ T cell receptor that is useful in the treatment of a cancer. The sequence may be similar to an endogenous γδ T cell receptor. The difference being that the exogenous immune receptor has been purposively selected for a specific target. The exogenous immune receptor is e.g., expressed from a transgene construct and not from endogenous loci. An exogenous immune receptor according to the disclosure may be of a different origin, i.e., from another species, as compared to the origin of the T cells that were engineered to provide for the engineered T cells with exogenous immune receptors. An exogenous immune receptor may be of the same origin, i.e., from the same species, as compared to the origin of the T cells that were engineered to provide for the engineered T cells with exogenous immune receptors. An exogenous immune receptor may also be an engineered γδ T cell receptor or an engineered αβ T cell receptor.

An engineered T cell receptor is a T cell receptor of which the amino acid sequence has been modified such that it has a different amino acid sequence as compared to the corresponding amino acid sequence of an endogenous T cell receptor, i.e., at least not taking into account the CDRs thereof. Hence, the modification as present in engineered T cell receptors should not interfere with the original antigen specificity. Such engineering involves modifying the amino acid sequence of e.g., the constant region of one or both of the T cell receptor chains.

Any of the immune receptors according to the disclosure may also be a chimeric antigen receptor (CAR). Chimeric antigen receptors (CARs) are recombinant receptors that combine the specificity of an antigen-specific antibody with the T-cell's activating functions (as reviewed Shi et al., Mol Cancer. 2014 Sep. 21; 13:219). A CAR may be a fusion molecule between an antibody and a trans-membrane domain allowing expression of an antibody at the cell surface of an immune cell as well as signaling into the cell.

In one aspect of the disclosure, any of the immune receptors according to the disclosure may be selected from the group consisting of an (engineered) γδ T cell receptor, an (engineered) αβ T cell receptor, or a chimeric antigen receptor (CAR).

Enrichment of Engineered Immune Cells

Also provided is a negative selection method, i.e., a method for obtaining a preparation of immune cells with exogenous immune receptors, comprising the steps of:

a) providing a mixture of immune cells comprising
immune cells expressing exogenous immune receptors according to the disclosure, wherein preferably expression of endogenous immune receptors is suppressed; and
immune cells with endogenous immune receptors;

b) contacting the mixture of immune cells with an antibody that specifically binds to the endogenous immune receptor, preferably a BW242/412 antibody, to allow formation of an antibody-immune cell with endogenous immune receptor complex; and

c) removing the antibody-immune cell with endogenous immune receptor complex from the mixture of immune cells to thereby obtain a preparation of immune cells expressing exogenous immune receptors.

As an alternative, also provided is a positive selection method, i.e., a method for obtaining a preparation of immune cells with exogenous immune receptors, comprising the steps of:

a) providing a mixture of immune cells comprising
immune cells expressing exogenous immune receptors according to the disclosure, wherein preferably expression of endogenous immune receptors is suppressed; and
immune cells with endogenous immune receptors;

b) contacting the mixture of immune cells with an antibody that specifically binds to the exogenous immune receptor, preferably an H57-597 antibody, to allow formation of an antibody-immune cell with exogenous immune receptor complex; and

c) obtaining the antibody-immune cell with exogenous immune receptor complex from the mixture of immune cells to thereby obtain a preparation of immune cells expressing exogenous immune receptors.

An advantage of positive selection can be in release approaches (combinations and several positive selections one after another). Also, it could allow selection of high transgene expressers, which may have an advantage toward purity and recovery.

However, the negative selection method is particularly preferred because it allows to select for the engineered T cells without requiring any interference with the engineered T cell. The engineered T cell can remain untouched. It was realized that by selecting engineered T cells using positive selection methods, e.g., using a selection marker, still, subpopulations of engineered T cells may exist that express functional levels of endogenous alpha beta T cell receptors in addition to expressing the exogenous immune receptor of the desired specificity and the separate selection marker. Such subpopulations will be selected in any positive selection strategy and can limit the therapeutic efficacy and safety of engineered cell products.

Furthermore, positive selection methods for selecting engineered T cells using e.g., an antibody that binds to the exogenous immune receptor may induce apoptosis in a substantial number of transduced cells. In addition, positive selection methods that include selection markers requires the addition of genes that can induce an unwanted immune response as such selection markers typically are non-host (e.g., non-human) and therefore may be recognized as being foreign resulting in elimination of transduced cells by the host. More importantly, ligation of an antibody to the transgene product in vitro can lead to T cell activation and possibly activation induced cell death (as well as the complement mediated killing and Fc receptor expressing cell killing).

The disclosure thus provides for methods that allow for enrichment of engineered T cells without requiring the addition of any additional genes.

A novel strategy was developed, that in addition to selecting the engineered T cells, also may eliminate unwanted subpopulations as described above, do not require any additional genes to be included in the engineered T cell except for the exogenous immune receptor, and that allow the engineered T cells to remain untouched.

Hence, in contrast to any of the selection methods as used in the prior art that use e.g., selection markers, or any of the selective killing methods used in the prior art that use e.g., suicide genes, the current disclosure now provides for modified exogenous immune receptors that do not require any additional selection marker genes and/or any additional suicide genes. The disclosure now allows for the production of engineered T cells that can be enriched for in an untouched manner, i.e., in the negative selection method, the engineered T cells do not require any binding or interaction with any outside agent such as e.g., an antibody.

In the first step of the negative selection method, a mixture of T cells is provided that comprises engineered T cells with exogenous immune receptors and T cells that express an endogenous αβ T cell receptor. Such a mixture of T cells can be prepared as described further below. This mixture of T cells is contacted with an antibody that specifically binds to the endogenous alpha beta T cell receptor, to allow formation of an antibody-non-engineered T cell complex. The antibody that specifically binds to the endogenous alpha beta T cell receptor does not bind specifically to the exogenous immune receptor. Hence, the antibody is selective for the endogenous alpha beta T cell receptor.

An antibody that specifically binds to an alpha beta T cell receptor binds, for example, to the alpha chain of the T cell receptor, the beta chain of the T cell receptor, particularly Domain 3 thereof, or both the alpha and beta chain of the T cell receptor. Examples of the extracellular domains of alpha and beta chains of human origin are respectively listed in SEQ ID NO:12-13. As said, alpha beta T cell receptors have variable domains, with the most variable regions constituted by the CDRs of the alpha and beta chains. As said, endogenous alpha beta T cell receptors of the non-engineered T cells are heterogeneous with regard to specificity, the antibody that specifically binds to the endogenous alpha beta T cell receptor binds with heterogeneous populations of alpha beta T cell receptors. Hence, the antibody specifically binds to regions of the alpha beta T cell receptor that are found in heterogeneous populations of alpha beta T cell receptors. Preferably, the antibody specifically binds to the constant regions of the alpha beta T cell receptor. Preferably, the antibody specifically binds to the constant region of the human alpha chain, and/or to the constant region of the human beta chain. Preferably, the antibody preferably binds to the constant region of the human alpha chain as listed for SEQ ID NO:12, and/or to the constant region of the human beta chain, as listed for SEQ ID NO:13.

Binding of an antibody that specifically binds to the alpha beta T cell receptor can be detected e.g., via FACS analysis. For example, non-engineered T cells are contacted with either a control antibody or an antibody that specifically binds to the alpha beta T cell receptor. An antibody that specifically binds to the alpha beta T cell receptor according to the disclosure can be defined as being an antibody that results in an increase of mean-fluorescence intensity (MFI), relative to the control antibody, as determined by flow cytometry. The MFI is the mean of the fluorescence intensity in the fluorescence channel that is chosen (FITC, PE, PerCP, etc.). As a negative control antibody, an antibody that does not bind to immunoglobulins (or to a very different immunoglobulin) may be used. Hence, the skilled person is well capable of selecting appropriate conditions to determine specific binding of an antibody to the alpha beta T cell receptor. Antibody binding can be expressed in terms of specificity and affinity. The specificity determines which antigen or epitope thereof is bound by the antibody. The affinity is measure of the strength of the binding between an antibody and the antigen (Kₐ).

The person skilled in the art is thus well capable of selecting an antibody that specifically binds to the endogenous alpha beta T cell receptor. For example, an antibody that specifically binds to the human endogenous alpha beta T cell receptor is available commercially from Miltenyi (Miltenyi Biotec GmbH, Friedrich-Ebert-Straße 68, 51429 Bergisch Gladbach, Germany). This antibody is from cell clone BW242/412, which is of the mouse isotype IgG2b. A FITC labelled BW242/412 antibody is available from Miltenyi under order no. 130-098-688. The BW242/412 cell clone and the antibody expressed by BW242/412 is described in detail i.a. EP0403156B1. In particular, such an antibody is an antibody as encoded by the BMA031 heavy and light chain sequence as listed for clone BMA031 in EP0403156B1. Other suitable antibodies are e.g., anti-αβTCR antibodies as available from Beckman Coulter, Marseille Cedex, France, for example, pan-αβTCR-PE (#A39499) or pan-αβTCR-PC5 (#A39500). A further suitable antibody for mouse alpha beta chains or the 9/11 modified Domain 3 as described herein, may be the murine TCRβ-PE (clone H57-597) available from BD Pharmingen (BD, 1 Becton Drive, Franklin Lakes, N.J. USA)

After formation of the antibody-non-engineered T cell complex, next the antibody-non engineered T cell complex is separated from the mixture of T cells to thereby obtain a preparation enriched in engineered T cells. This way, the non-engineered T cells with endogenous alpha beta T cell receptors are removed from the mixture of T cells. Suitable separation steps using specific antibodies are well known in the art. For example, magnetic activated cell sorting (MACS), fluorescent activated cell sorting (FACS), or immunoaffinity chromatography are methods that may be used. The antibody that specifically binds to the alpha beta T cell receptor may be coupled to magnetic beads for MACS, or fluorescently labelled for FACS, or coupled to a suitable chromatography resin. With MACS or immunoaffinity chromatography, the cells that do not bind to the resin are obtained thereby obtaining a preparation enriched in engineered T cells. In FACS, the cells that are not labelled are obtained, thereby obtaining a preparation enriched in engineered T cells. As an alternative to using only the antibody that specifically binds to the alpha beta T cell receptor, instead, secondary antibodies may be used that are specific for the antibody. For example, when the antibody is a mouse antibody, a goat-anti-mouse antibody coupled to a resin or magnetic bead may be used. The antibody-non-engineered T cell complex will bind to the resin or magnetic bead via the goat-anti-mouse antibody. Or, the antibody that specifically binds to the alpha beta T cell receptor may carry a biotin label such as described in the examples, and an anti-biotin antibody coupled to a resin or magnetic bead may be used. Hence, many separation methods are available and well known to the skilled person that may be suitable for separating the antibody-non engineered T cell complex from the mixture of T cells to thereby obtain a preparation enriched in engineered T cells.

As said, the mixture of T cells may also comprise engineered T cells that have a suboptimal expression of the exogenous immune receptor and that may still have a substantial amount of endogenous alpha beta T cell receptor expressed. Hence, the mixture of T cells that is provided may comprise engineered T cells with exogenous immune receptors, non-engineered T cells with endogenous alpha beta T cell receptors, and engineered T cells with exogenous immune receptors and endogenous alpha beta T cell receptors. Thus, in the negative selection method, non-engineered T cells with endogenous alpha beta T cell receptors, and engineered T cells with exogenous immune receptors and endogenous alpha beta T cell receptors may also be separated from the mixture. Hence, the separation step is not limited to only separating endogenous alpha beta T cells from the mixture. Thus, when in step a) of the negative selection method, a mixture of T cells is provided, this mixture may also comprise such engineered T cells with exogenous immune receptors and endogenous alpha beta T cell receptors. In step b) an antibody-engineered T cell complex may than be formed via the endogenous alpha beta T cell receptor to allow for separation of these cells in step c) in addition to the non-engineered T cell cells.

As said, in the methods of the disclosure a mixture of immune cells is provided comprising engineered immune cells with exogenous immune receptors and non-engineered immune cells with endogenous alpha beta T cell receptors. In one embodiment of the disclosure, providing the mixture comprises the steps of
i. providing immune cells, preferably T cells;
ii. providing a nucleic acid or nucleic acids encoding the exogenous immune receptor;
iii. introducing the nucleic acid or nucleic acids into the immune cells to thereby provide a mixture of immune cells comprising immune cells with exogenous immune receptors and immune cells with endogenous immune receptors.

The step of providing T cells may comprise providing alpha beta T cells, e.g., via selecting cells using MACS selection using e.g., an alpha beta T cell receptor antibody such as BW242. The step of providing T cells may also comprise providing PBMCs that comprise T cells including gamma and delta T cells and alpha beta T cells. The step of providing T cells may also comprise providing a mixture of cells comprising alpha beta T cells and gamma delta T cells, e.g., T lymphocytes via MACS selection with a CD3 antibody. The T cells may be primary cells, for example, from a subject, such as a human subject. Any cell type, being a primary cell or any other cell line will suffice, as long as the cell population, or a substantial part thereof, comprises cells expressing an alpha beta T cell receptor, i.e., being positive for the αβT-cell receptor in e.g., a FACS sorting.

An (exogenous) immune receptor according to the disclosure may comprise a first chain and a second chain. These may be provided on a single nucleic acid or on two separate nucleic acids. A first nucleic acid encoding the first chain, and a second nucleic acid encoding the second chain, or a single nucleic acid encoding both the first and second chains. The nucleic acid or nucleic acids may be DNA or RNA. As long as when it is introduced in a cell and expressed such that the amino acid sequence of the exogenous immune receptor it encodes is expressed on the surface of the cell.

Preferably, in one embodiment, the nucleic acid encoding the exogenous immune receptor encodes an exogenous immune receptor wherein the different chains are expressed as a single translated protein product that comprising the F2A or T2A peptide linker sequence in between the encoding sequences of the both chains resulting in self-cleavage of the translated protein such that separate chains are formed.

The nucleic acid or nucleic acids that encode the exogenous immune receptor may be mRNA that can be translated directly in the exogenous immune receptor when introduced in the cytoplasm of a T cell, e.g., via transfection. Preferably, the nucleic acid (or nucleic acids) encoding e.g., a T cell receptor chain is comprised in a genetic construct. The genetic construct (or constructs) allows the expression of mRNA that encodes the exogenous immune receptor such that it is expressed on the surface of the engineered T cell. A genetic construct may be comprised in a DNA vector or in a viral vector. Introduction of the nucleic acid or nucleic acids may be via transfection or transduction methods depending on what type of nucleic acid or nucleic acids are used. It is understood that depending on what type of genetic construct or constructs are used, the genetic construct may consist of DNA or RNA. For example, when a genetic construct is incorporated in a retroviral or lentiviral vector the genetic construct is comprised in an RNA vector genome (i.e., the sequence that encodes the genetic construct). Retroviral and lentiviral vectors are well known in the art having an RNA genome that, when entered in a cell, is reverse transcribed into DNA that is subsequently integrated into the host genome. Reverse transcription thus results in the genetic information, i.e., the genetic construct, being transformed from RNA into double stranded DNA thereby allowing expression therefrom. Integration is advantageous as it allows proliferation of transduced cells while maintaining the viral vector genome comprising the genetic construct. A genetic construct may also be comprised in a DNA vector, e.g., plasmid DNA. A suitable DNA vector may be a transposon. Suitable transposon systems (e.g., class I or class II based) are well known in the art. As said, when an exogenous immune receptor comprises two chains, e.g., a gamma and delta T cell receptor chain, two separate genetic constructs can be provided e.g., on a single or two separate retroviral or DNA vectors. Alternatively, a single genetic construct may also express a single mRNA encoding the two chains, such as described in the example section. Such an mRNA may encode the two chains separately, e.g., via an IRES, or via using self-cleavable peptide sequences as described herein.

The nucleic acid or nucleic acids that are used provide for expression of the encoded exogenous immune receptor. This is achieved e.g., via high levels of expression of the exogenous immune receptor by using e.g., a strong promoter. Using high expression levels results in suppression of endogenous T cell receptor expression as exemplified in the example section. Endogenous T cell expression may also be suppressed via alternative and additional methods such as e.g., RNAi via shRNA expression, zinc fingers, CRISPR, or TALENS.

In any case, introducing the nucleic acid or nucleic acids into the T cells may be efficient but may provide for a mixture of T cells comprising engineered T cells with exogenous immune receptors and non-engineered T cells with endogenous alpha beta T cell receptors. The non-engineered T cells with endogenous alpha beta T cell receptors representing T cells in which no nucleic acid or nucleic acids was introduced. Also, as said, the engineered T cells may also comprise a subpopulation of engineered T cells that is also present in the mixture of T cells wherein the introduction did not result in (sufficient) suppression of endogenous alpha beta T cell receptors. Such a subpopulation of T cells that do not have (sufficient) suppression of endogenous alpha beta T cell receptors may also be efficiently removed from the mixture of T cells because the anti-alpha beta T cell receptor antibody may bind thereto. Such a population of engineered T cells may be positively stained for the exogenous immune receptor and the endogenous alpha beta T cell receptor in e.g., a FACS analysis.

The engineered T cells comprising exogenous immune receptor may also comprise selectable markers. A selectable marker may be defined as any nucleic acid sequence and/or amino acid sequence in addition to the exogenous immune receptor that allows cells that are provided therewith to be selected. For example, selectable markers may be neomycin or puromycin resistance genes. Selection of cells to which the genetic construct and/or vector has been transferred may than be performed by incubating in the presence of neomycin or puromycin. Other selectable markers may be, for example, any one of green, red and yellow fluorescent proteins. Selection may then be performed by using e.g., FACS. As said above, non-engineered T cells that are the result of insufficient suppression of endogenous alpha beta T cell receptors may comprise the genetic construct and thus also a selectable marker. Such cells are not desirable and removing these will also result in an enrichment of engineered T cells. Hence, the present enrichment method is also of benefit to engineered T cells of the prior art that have been selected with a positive selection method e.g., by inclusion of an additional selection marker and/or by selecting cells with an antibody directed against the exogenous immune receptor.

However, it is not required to have a selectable marker, as the negative selection method of the disclosure allows to remove non-engineered cells without using any selectable marker. It is understood that according to the disclosure, the selectable marker is not the exogenous immune receptor. Thus, in one aspect of the disclosure, the engineered T cells do not separately express a selectable marker. Accordingly, the nucleic acid or nucleic acids according to the disclosure do(es) not require to encode a separately expressed selection marker in addition to encoding the exogenous immune receptor. Hence, in one embodiment, the nucleic acid or acids, or DNA vectors, retroviral vectors, lentiviral vectors, transposons or the like, that encode the exogenous immune receptor do not comprise a selectable marker. It is understood selectable marker are to be functional in the engineered T cells.

In another aspect of the disclosure, the mixture of T cells comprising non-engineered and engineered T cells are human cells. Hence, this means that nucleic acid or nucleic acids encoding an exogenous immune receptor are introduced in human T cells to provide for such mixture of T cells. This means that the antibody used in the negative selection method specifically binds to the human alpha beta T cell receptor. In a further aspect of the disclosure, the antibody that specifically binds to the human alpha beta T cell receptor is a BW242/412 antibody. As said, the antibody is commercially available from Miltenyi (Miltenyi Biotec GmbH, Friedrich-Ebert-Straße 68, 51429 Bergisch Gladbach, Germany) and described in detail i.a. in EP0403156B1.

As is clear from the above, in the negative selection method, the antibody that specifically binds to the endogenous alpha beta T cell receptor does not specifically bind to the exogenous immune receptor. Hence, these selection criteria apply for any antibody that may be selected for the negative selection method. The exogenous immune receptor can therefore preferably not correspond to an alpha beta T cell receptor that is endogenous to the T cells used, albeit provided as a transgene. This is because otherwise in steps b) and c) of the negative selection method not only non-engineered T cells are removed but engineered T cells are removed as well. In case it is desirable to use an alpha beta T cell receptor as an exogenous immune receptor it is thus preferred to modify the sequence thereof according to the disclosure such that the antibody no longer binds specifically to the exogenous immune receptor. Hence, in one aspect of the disclosure, the exogenous immune receptor is an engineered alpha beta T cell receptor.

In one aspect of the disclosure, the exogenous immune receptor is an engineered gamma delta T cell receptor or an engineered alpha beta T cell receptor. In one aspect of the disclosure, the exogenous immune receptor is of the same origin of the mixture of T cells. In another aspect, the exogenous immune receptor is a human engineered gamma delta T cell receptor or a human engineered alpha beta T cell receptor. In contrast to the alpha beta T cell receptor, the gamma delta T cell receptor has a sequence that is different from the alpha beta T cell receptor. Hence, an antibody specifically binding to the endogenous alpha beta T cell receptor normally does not specifically bind any endogenous gamma delta T cell receptor. Hence, it is not be required to modify a gamma delta T cell receptor that is used as an exogenous immune receptor. Nevertheless modifying a gamma delta T cell receptor may be contemplated for other reasons, e.g., when the engineered T cells are used in vivo and are to be differentiated from endogenous gamma delta T cells, as further explained below.

In one embodiment, the exogenous immune receptor is an engineered gamma delta T cell receptor comprising part of the gamma and delta chain sequences as listed in SEQ ID NO:14 and SEQ ID NO:15. These sequences correspond to G115 and δ5. Engineered T cells with this exogenous immune receptor may be enriched for by negative selection using e.g., the BW242 antibody.

In another aspect of the disclosure, the engineered alpha beta T cell receptor or engineered gamma delta T cell receptor comprises a modified constant region, i.e., a modified sequence according to the disclosure. Modifying the constant region may be advantageous as any risk of affecting the variable region and thus antigen specificity and/or affinity may be avoided.

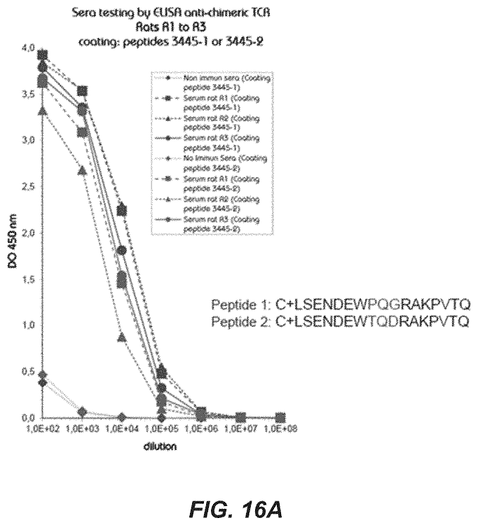

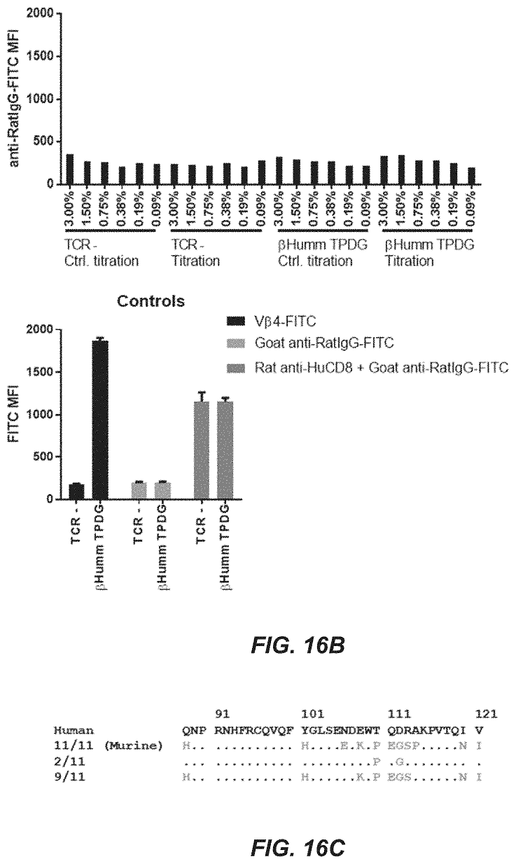

In one embodiment, in the negative selection method according to the disclosure, the antibody that specifically binds to the human alpha beta T cell receptor is a BW242/412 antibody and the exogenous immune receptor is an engineered human alpha beta T cell receptor. Preferably, the engineering comprises modification of the constant region of the human alpha beta T cell receptor. More preferably, the modification constant region comprises modification of the Domain 3 of the T cell receptor beta chain, wherein preferably the modification comprises murinization of Domain 3. The binding site of the BW242/412 antibody was mapped to Domain 3 of the T cell receptor beta chain. Hence, modifying only this region will allow to the BW242/412 antibody to be selective for the human endogenous alpha beta T cell receptor while not substantially binding to the exogenous immune receptor, i.e., the modified human alpha beta T cell receptor chain. Preferably, the alpha beta T cell receptor chain comprises the specific murine amino acid modifications according to the murine Domain 3 in the human Domain 3 of the human beta T cell receptor as depicted in FIG. FIG. 16C (see amino acids of the human sequence as aligned with the corresponding mouse sequence).

Accordingly, by mapping the binding site of the antibody that binds to the endogenous alpha beta T cell receptor, the modification of the corresponding human alpha beta T cell receptor may be minimized according to the disclosure. The binding site of the BW242/412 antibody is now mapped to Domain 3, and selectively modifying the amino acids of Domain 3 has identified the minimum amino acid modifications of Domain 3 to abrogate binding of a BW242/412 antibody. This way, a minimally modified engineered human alpha beta T cell receptor may be provided differing only in a few amino acids thereby minimizing immunogenic effects and/or obtaining improved persistence of engineered immune cells when administered to a patient, in comparison with a more or fully murinized sequence. Likewise, the same approach may be followed when antibodies other than BW242 are to be selective between an endogenous alpha beta T cell receptor and a corresponding engineered alpha beta T cell receptor.

Enriched Engineered Immune Cells and Their Uses

In another embodiment, the methods according to the disclosure as described above provide for a preparation of engineered immune cells obtainable by any one of the negative of positive selection methods. Such a preparation will comprise a higher percentage of engineered immune cells as compared to a preparation not subjected to the method, for example, at least 30, 40, 50, 60, 70, 80, 90, 95, 99 or 100% of the immune cells in the preparation are engineered immune cells. A preparation of enriched engineered T cells as obtainable by the negative selection method may also be defined as a preparation of enriched engineered T cells from which non- and poorly engineered T cells with endogenous alpha beta T cell receptors have been separated using an antibody specifically binding to the endogenous alpha beta T cell receptor. Such a preparation may also be defined as a preparation of enriched engineered T cells wherein the enriched engineered T cells do not substantially comprise an endogenous alpha beta T cell receptor. Such a preparation may also be defined as a preparation of enriched engineered T cells wherein the enriched engineered T cells do not substantially comprise an endogenous alpha beta T cell receptor and also do not comprise a selectable marker. Such a preparation may also be defined as a preparation of enriched engineered T cells wherein the enriched engineered T cells do not substantially comprise an endogenous alpha beta T cell receptor and also do not comprise a selectable marker and have not been selected with an antibody that binds with the exogenous immune receptor.

The preparations of enriched engineered T cells show an enhanced killing of cancer cells when compared with preparations that are enriched using positive selection methods using selectable marker(s). When T cells are provided with an exogenous immune receptor that provides specificity to a particular cancer such cells will be selectively killed when a subject is provided with the preparation enriched in the engineered T cells. Such preparations enriched in engineered T cells according to the disclosure are therefor in particularly useful in medical treatments. Medical treatments that can be contemplated are e.g., the treatment of a cancer. As the engineered T cells no longer require the expression of a selection marker, any adverse event relating to the expression of a selection marker can be avoided. Furthermore, the enriched engineered T cells will have most, if not all, of the T cells expressing endogenous alpha beta T cell receptors removed and therefore any risk of endogenous alpha beta T cell receptors causing unwanted targeting may be avoided in the negative selection method. The enriched engineered T cells will also not suffer from any cell death that is associated with binding of an antibody to the exogenous immune receptor that may also be detrimental to the quality of the enriched engineered T cell product that is administered.

Depletion of (Enriched) Engineered T Cells In Vivo

In another embodiment, an antibody that specifically binds to a modified exogenous immune receptor, according to the disclosure, is provided for use in the treatment of subjects that suffer from adverse events when treated with a preparation enriched in engineered T cells with the exogenous immune receptor obtainable by any one of the methods above. As explained above, enriched engineered T cells obtainable by any one of the methods of the disclosure are useful in medical treatments. Nevertheless, such a treatment may in some cases lead to adverse side effects due to the enriched engineered T cells that were administrated. Side effects may be uncontrolled proliferation or activation, or activation against unpredicted antigens on healthy cells e.g., of the subject. Hence, in such a scenario, it is desirable to selectively eliminate (deplete) the engineered T cells that were administered to the subject. This can be achieved by administering an antibody that specifically targets the engineered T cells, i.e., comprising the exogenous immune receptor. The antibody does not target endogenous T cells, such as endogenous alpha beta T cells or endogenous gamma delta T cells. This way, it is no longer required to include in engineered T cells in addition to the exogenous immune receptor a further genetic construct encoding e.g., a suicide gene or other gene that allows for selectively killing engineered T cells.

As said, antibody is not to target the endogenous T cells, in case engineered alpha beta T cell receptors or engineered gamma delta T cell receptors are used having an origin corresponding to endogenous T cell receptors, the exogenous immune receptors can be modified according to the disclosure, i.e., engineered, such that the antibody only targets the exogenous immune receptor. For example, when an exogenous immune receptor is used that has the human Domain 3 region replaced with the mouse Domain 3 region, the antibody e.g., derived from H57-597 derived from HB-218 (ATCC) is to target the mouse Domain 3 region. This way, in a human subject, the antibody will selectively target the engineered exogenous immune receptor and will not target the endogenous T cell receptor. Hence, mapping the binding sites of e.g., antibodies that bind mouse alpha beta T cell receptors or mouse gamma delta T cell receptors is useful as it will provide for the specific regions (or even specific antibodies) of the respective T cell receptors that can be transferred to a corresponding human T cell receptor. Optimally, as exemplified with the mentioned antibodies BW242/412 (used for enrichment) and H57-597 (used for depletion) the modified region used for enrichment is identical in sequence with the sequence used for depletion such as the region derived from mouse Domain 3 region. This region may be in particular interesting due to its prominent location in the T cell receptor as well as potential to be immunogenic, Likewise, when a chimeric antigen receptor is used, which may be built from components that may be identical to host proteins (e.g., derived from host antibodies and/or derived from host CD3) the antibody is selected not to target the corresponding host proteins but only the chimeric antigen receptor. In a same approach as described for the alpha beta TCR such host sequences may be modified, i.e., engineered, as well, such that the antibody administered does not target the host proteins. Hence, in case a chimeric antigen receptor is used, it may be an engineered chimeric antigen receptor in the sense that parts of the host sequences may be modified such that the antibody that is used can differentiate between the engineered CAR and corresponding host protein sequences. Hence, by aligning, for example, mouse and human sequences, and by comparing mouse and human immune receptors with regard to binding of an antibody such as described in the examples, a human immune receptor may be murinized, i.e., parts of the human immune receptor may be exchanged for a corresponding part of the murine receptor as proposed by the disclosure. Murinization may thus involve replacing a part of the sequence of an immune receptor by a corresponding part of murine origin, such a part may e.g., be a stretch of 10-50 amino acids, but a part (or parts) may also comprise one or more amino acids that are part of the regions that are corresponding and that differ between the two sequences, preferably the specific modifications as disclosed herein.

Preferably, the treatment of subjects involves the treatment of humans, wherein preferably, the antibody is a human antibody or e.g., variable domains derived from non-human antibodies such as the H57-597 antibody, are engineered into a human antibody backbone via humanization. Preferably, a human antibody is used because non-human sequences may invoke unwanted responses, e.g., in case of a mouse antibody, a human-anti-mouse response may be triggered, which is not desirable. It is understood that the term human antibody also includes humanized antibodies.

Preferably, the antibody for use according to the disclosure is in combination with a drug conjugate, preferably a cell cycle inhibitor, more preferably monomethyl auristatin E (MMAE). The antibody-drug conjugate (ADC) may be the antibody combined or linked to a cytotoxic agent or cytotoxin (e.g., anti-cancer agent). The antibody and/or the drug conjugate may induce cell death of the immune cells with the exogenous immune receptor.

As said, the administering of the antibody, or alternatively, an immune cell expressing a CAR according to the disclosure (i.e., having an antigen binding domain comprising an amino acid sequence having at least 70, 80, 90, or 100% sequence identity with SEQ ID NO:1; and/or an amino acid sequence having at least 70, 80, 90, or 100% sequence identity with SEQ ID NO:2) may induce selective killing of the engineered T cells (expressing a murine derived epitope as described herein). Persisting engineered CAR T cells that have been used to eliminate engineered T cells can be also used as adaptor cells for soluble fragments (comprising a murine derived epitope as described herein) binding to the same epitope as the engineered T cells, e.g., an epitope on a target (cancer) cell. By modulating affinities of adaptors, affinities can be later exchanged between two different adaptors (e.g., adaptor of 9 versus 11 murine derived AA as described herein). As an example: a construct bearing the CAR T format H57-597 can eliminate abTCR cells harboring the murine epitope and at the very same time be used as docking side for novel CAR T. So that one can infuse the cells to eliminate existing CAR T and then later on take advantage of the persisting cell and infuse a novel adaptor.

Additionally, one can generate different adaptors with different affinities that replace each other using different length of 9 and 11 murine AA to generate different affinities. For example, it is possible to first use an adaptor with the 11 murine derived amino acids as disclosed herein and later an adaptor with the 9 amino acids as disclosed herein, which should outcompete the previous one.

Such selective killing may be inducing death after binding of the antibody to the exogenous immune receptor. Such selective killing may be induced directly or indirectly. Human derived sequences of the antibody backbone is preferred in the treatment of humans because selective killing may include antibody-dependent cell-mediated cytotoxicity (ADCC), complement-dependent cytotoxicity (CDC), or direct apoptosis.

With regard to the medical uses as described above with regard to the use of antibodies that target engineered T cells, such a medical use is not restricted to preparations enriched in engineered T cells as obtainable by the methods of the disclosure as described above. Such a medical use may also be applied to any engineered T cell, provided that the antibody that is used specifically binds to the exogenous immune receptor and not to immune receptors of the host or host protein sequences. Such engineered T cells may also be enriched for by using prior art methods that use e.g., a selection marker. Furthermore, as the method for selecting the modified T cells is not required, the use is also applicable in engineered NK cells with exogenous immune receptors.

Hence, in another embodiment, an antibody is provided that specifically binds to an exogenous immune receptor, for use in the treatment of subjects that suffer from adverse events when being treated with engineered lymphocytes with the exogenous immune receptor. Preferably, the exogenous immune receptor is an engineered immune receptor. Preferably in combination with a drug conjugate, preferably a cell cycle inhibitor, more preferably monomethyl auristatin E (MMAE). The antibody-drug conjugate (ADC) may be the antibody combined or linked to a cytotoxic agent or cytotoxin (e.g., anti cancer agent). The antibody and/or the drug conjugate may induce cell death of the immune cells with the exogenous immune receptor.

Preferably, the subjects are human. Preferably, the engineered lymphocytes are human engineered lymphocytes. Preferably, the engineered lymphocytes are engineered NK cells or engineered T cells. The antibody most preferably is a human antibody or a humanized antibody, which preferably induces cell death of the engineered lymphocytes with the exogenous immune receptor as described above.

Definitions

In the disclosure, a number of terms are used. In order to provide a clear and consistent understanding of the specifications and claims, including the scope to be given to such terms, the following definitions are provided. Unless otherwise defined herein, all technical and scientific terms used have the same meaning as commonly understood by one of ordinary skill in the art to which this disclosure belongs.

Methods of carrying out the conventional techniques used in methods of the disclosure will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N.Y., 1989; Ausubel et al., Current Protocols in Molecular Biology, John Wiley & Sons, New York, 1987 and periodic updates; and the series Methods in Enzymology, Academic Press, San Diego.

In this document and in its claims, the verb “to comprise” and its conjugations is used in its non-limiting sense to mean that items following the word are included, but items not specifically mentioned are not excluded. It encompasses the verbs “consisting essentially of” as well as “consisting of.”

As used herein, the singular forms “a,” “an” and “the” include plural referents unless the context clearly dictates otherwise. For example, a method for isolating “a” DNA molecule, as used above, includes isolating a plurality of molecules (e.g., 10s, 100s, 1000s, 10s of thousands, 100s of thousands, millions, or more molecules).

Aligning and alignment: With the term “aligning” and “alignment” is meant the comparison of two or more nucleotide sequences based on the presence of short or long stretches of identical or similar nucleotides. Several methods for alignment of nucleotide sequences are known in the art, as will be further explained below. With the term “aligning” and “alignment” is also meant the comparison of two or more amino acid sequences based on the presence of short or long stretches of identical or similar amino acids. Several methods for alignment of amino acid sequences are known in the art, as will be further explained below.

“Expression of a gene” (or protein or immune receptor) refers to the process wherein a DNA region, which is operably linked to appropriate regulatory regions, particularly a promoter, is transcribed into an RNA, which is biologically active, i.e., which is capable of being translated into a biologically active protein or peptide (or active peptide fragment) or which is active itself (e.g., in posttranscriptional gene silencing or RNAi). An active protein in certain embodiments refers to a protein being constitutively active. The coding sequence is preferably in sense-orientation and encodes a desired, biologically active protein or peptide, or an active peptide fragment.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary join two or more protein encoding regions, contiguous and in reading frame.

The term “genetic construct” means a DNA sequence comprising a region (transcribed region) that is transcribed into an RNA molecule (e.g., an mRNA) in a cell, operably linked to suitable regulatory regions (e.g., a promoter). A genetic construct may thus comprise several operably linked sequences, such as a promoter, a 5′ leader sequence comprising e.g., sequences involved in translation initiation, a (protein) encoding region, splice donor and acceptor sites, intronic and exonic sequences, and a 3′ non-translated sequence (also known as 3′ untranslated sequence or 3′UTR) comprising e.g., transcription termination sequence sites.

“Identity” is a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER; Gribskov, M. and Devereux, J., eds., M Stockton Press, New York, 1991). While a number of methods exist to measure identity between two polynucleotide or polypeptide sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in GUIDE TO HUGE COMPUTERS, Martin J. Bishop, ed., Academic Press, San Diego, 1994, and Carillo, H., and Lipton, D., SIAM J. Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J. Molec. Biol. (1990) 215:403). Similarly, by a polypeptide having an amino acid sequence having at least, for example, 95% “identity” to a reference amino acid sequence of SEQ ID NO:1 is intended that the amino acid sequence of the polypeptide is identical to the reference sequence except that the polypeptide sequence may include up to five amino acid alterations per each 100 amino acids of the reference amino acid of SEQ ID NO:1. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence.

As used herein, the term “promoter” refers to a nucleic acid sequence that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. Optionally the term “promoter” includes herein also the 5′ UTR region (5′ Untranslated Region) (e.g., the promoter may herein include one or more parts upstream (5′) of the translation initiation codon of a gene, as this region may have a role in regulating transcription and/or translation).

The terms “amino acid sequence” or “protein” or “peptide” refer to molecules consisting of a chain of amino acids, without reference to a specific mode of action, size, 3 dimensional structure or origin. A “fragment” or “portion” of thereof may thus still be referred to as an “amino acid sequence” or “protein” or “peptide.”

With the term “conservative substitution” of a specific amino acid is meant a substitute amino acid that does not change the desired activity of the polypeptide, or immune receptor, e.g., with respect to abrogation of binding to the BW242/412 antibody and/or binding to the H57-597 antibody. The skilled person knows that polypeptides having a different amino acid sequence can have the same activity. It is common general knowledge that it may be possible to substitute a certain amino acid by another one, without loss of activity of the polypeptide e.g., immune receptor. For example, the following amino acids may be exchanged for one another:

Ala, Ser, Thr, Gly (small aliphatic, nonpolar or slightly polar residues)

Asp, Asn, Glu, Gln (polar, negatively charged residues and their amides)

His, Arg, Lys (polar, positively charged residues)

Met, Leu, Ile, Val (Cys) (large aliphatic, nonpolar residues)

Phe, Ty, Trp (large aromatic residues)
(refer, for example, to Schulz, G. E. et al, Principles of Protein Structure, Springer-Verlag, New York, 1979, and Creighton, T. E., Proteins: Structure and Molecular Principles, W.H. Freeman & Co., San Francisco, 1984). Preferred substitutions are those that are conservative, i.e., wherein the residue is replaced by another of the same general type. In making changes, the hydropathic index of amino acids may be considered (See, e.g., Kyte et al., J. Mol. Biol. 157, 105-132 (1982). It is known in the art that certain amino acids may be substituted by other amino acids having a similar hydropathic index or score and still result in a polypeptide having similar biological activity. In making such changes, the substitution of amino acids whose hydropathic indices are within ±2 is preferred, those that are within ±1 are more preferred, and those within ±0.5 are even more preferred. Similarly, select amino acids may be substituted by other amino acids having a similar hydrophilicity, as set forth in U.S. Pat. No. 4,554,101. In making such changes, as with the hydropathic indices, the substitution of amino acids whose hydrophilicity indices are within ±2 is preferred, those that are within ±1 are more preferred, and those within ±0.5 are even more preferred.

“Engineered cells” refers herein to cells having been engineered, e.g., by the introduction of an exogenous nucleic acid sequence or specific alteration of an endogenous gene sequence. An exogenous nucleic acid sequence that is introduced may comprise a wild type sequence of any species that may be modified. An engineered cell may comprise genetic modifications such as one or more mutations, insertions and/or deletions in an endogenous gene and/or insertion of an exogenous nucleic acid (e.g., a genetic construct) in the genome. An engineered cell may refer to a cell in isolation or in culture. Engineered cells may be “transduced cells” wherein the cells have been infected with e.g., an engineered virus. For example, a retroviral vector may be used, such as described in the examples, but other suitable viral vectors may also be contemplated such as lentiviruses. Non-viral methods may also be used, such as transfections or electroporation of DNA vectors. DNA vectors that may be used are transposon vectors. Engineered cells may thus also be “stably transfected cells” or “transiently transfected cells.” Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient, but may also be a stable transfection wherein cells can be selected that have the gene construct integrated in their genome.

The term “selectable marker” is a term familiar to one of ordinary skill in the art and is used herein to describe any genetic entity that, when expressed, can be used to select for a cell or cells containing the selectable marker. Selectable marker gene products confer, for example, antibiotic resistance, or another selectable trait or a nutritional requirement. Selectable markers such as well-known in the art include green fluorescent protein (GFP), eGFP, luciferase, GUS and the like.

“αβT cells” or “alpha beta T cells” may be defined with respect of function as T lymphocytes that express an αβTCR, which recognizes peptides bound to MHC molecules (major histocompatibility complex), which are expressed on the surface of various cells. MHCs present peptides derived from the proteins of a cell. When, for example, a cell is infected with a virus, the MHC will present viral peptides, and the interaction between the αβTCR and the MHC-complex activates specific types of T-cells that initiate and immune responses to eliminate the infected cell. Hence, αβT cells may be functionally defined as being cells capable of recognizing peptides bound to MHC molecules. αβT-cells may be identified using an antibody specific for the αβ T-cell receptor such as described below (e.g., the BW242 antibody that is specific for a human αβ TCR). αβT cells may be selected from peripheral blood, for example, via the CD3 antigen, as the large majority of T cells have the αβTCR. Such a selection will also include γδT-cells. From such selected cells, the nucleic acid (or amino acid) sequence corresponding to the αT-cell receptor chain and the βT-cell receptor chain may be determined. Hence, αβT-cells may also be defined as being cells comprising a nucleic acid (or amino acid) sequence corresponding to the αT-cell receptor chain and/or the βT-cell receptor chain.

“γδT cells” or “gamma delta T cells” represent a small subset of T cells for which the antigenic molecules that trigger their activation is largely unknown. Gamma delta T cells may be considered a component of adaptive immunity in that they rearrange TCR genes to produce junctional diversity and will develop a memory phenotype. However, various subsets may also be considered part of the innate immunity where a restricted TCR is used as a pattern recognition receptor. For example, Vγ9/Vδ2 T cells are specifically and rapidly activated by a set of non-peptidic phosphorylated isoprenoid precursors, collectively named phosphoantigens. γδT-cells may be identified using an antibody specific for the γδ T-cell receptor. Antibodies suitable for FACS are widely available. Conditions are selected, such as provided by the antibody manufacturer that allows the selection of negative and/or positive cells. Examples of antibodies that may be suitable are available from BD Pharmingen (BD, 1 Becton Drive, Franklin Lakes, N.J. USA), γδTCR-APC (clone B1, #555718) or as available from Beckman Coulter, pan-γδTCR-PE (clone IMMU510, # IM1418U). Also, from such selected cells, the nucleic acid (or amino acid sequence) sequence corresponding to the γT cell receptor chain and/or the δT cell receptor chain may be determined. Hence, γδT cells may also be defined as being cells comprising a nucleic acid (or amino acid) sequence corresponding to a γT-cell receptor chain and/or a 62T-cell receptor chain.

T cells, or T lymphocytes, belong to a group of white blood cells named lymphocytes, which play a role in cell-mediated immunity. T cells originate from hematopoietic stem cells in the bone marrow, mature in the thymus (that is where the T is derived from), and gain their full function in peripheral lymphoid tissues. During T-cell development, CD4⁻CD8⁻ T-cells (negative for both the CD4 and CD8 co-receptor) are committed either to an αβ (alpha beta) or γδ (gamma delta) fate as a result of an initial β or δ TCR gene rearrangement. Cells that undergo early β chain rearrangement express a pre-TCR structure composed of a complete β chain and a pre-TCRα chain on the cell surface. Such cells switch to a CD4⁺CD8⁺ state, rearrange the TCRα chain locus, and express an αβTCR on the surface. CD4⁻CD8⁻ T cells that successfully complete the y gene rearrangement before the β gene rearrangement express a γδTCR and remain CD4⁻CD8⁻. (Claudio Tripodo et al. Gamm delta T cell lymphomas Nature Reviews Clinical Oncology 6, 707-717 (December 2009). The T cell receptor associates with the CD3 protein to form a T cell receptor complex. T cells, i.e., expressing an αβTCR or a γδTCR, express the T cell receptor complex on the cell surface. The γδT-cells constitute about 1-5% of the total population of T cells. The extracellular region of a T cell receptor chain comprises a variable region. The variable region of a T cell receptor chain three complementarity determining regions (CDR1, CDR2, CDR3) are located. These regions are in general the most variable and contribute to diversity among TCRs. CDR regions are composed during the development of a T-cell where so-called Variable-(V), Diverse-(D), and Joining-(J)-gene segments are randomly combined to generate diverse TCRs. The constant region of a T cell receptor chain, i.e., being either an alpha, beta, gamma or delta chain, does not substantially vary. Similarly, the framework regions of a T cell receptor chain, i.e., being either an alpha, beta, gamma or delta chain, do not substantially vary either.

Natural Killer cells (NK cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation. NK cells do not express T-cell antigen receptors (TCR) or Pan T marker CD3 or surface immunoglobulins (Ig) B cell receptors, but they usually express the surface markers CD16 (FcγRIII) and CD56 in humans, NK1.1 or NK1.2 in C57BL/6 mice. Up to 80% of human NK cells also express CD8.

The term “antibody” as used herein and as known in the art refers to any polypeptide comprising an antigen-binding site with complementarity determining regions (CDR). The term includes, but is not limited to antibodies, monoclonal antibodies, monospecific antibodies, multispecific antibodies, humanized antibodies, chimeric antibodies, human antibodies, single chain antibodies, heavy chain only antibodies, llama antibodies, single domain antibodies and nanobodies (e.g., VHH). The term “antibody” may also include immunoglobulin fragments such Fab, F(ab′)2, Fv, scFv, Fd, dAb, and other antibody fragments or other constructs comprising CDRs that retain antigen-binding function. Typically, such fragments comprise an antigen-binding domain. The antibodies or fragments thereof may comprise any of the known antibody isotypes and their conformations, for example, IgA, such as IgA1 or IgA2, IgD, IgE, IgG, such as IgG1, IgG2a, IgG2b, IgG3, IgG4, or IgM class.

In general, a CAR as disclosed herein may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the disclosure, the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell.

Such a CAR may be also named “anti-tag” CAR or “adapterCAR” or “universal CAR” as disclosed e.g., in U.S. Pat. No. 9,233,125B2.

The haptens or tags may be coupled directly or indirectly to a polypeptide (the tagged polypeptide), wherein the polypeptide may bind to the disease associated antigen expressed on the (cell) surface of a target. The tag may be e.g., a hapten such as biotin or fluorescein isothiocyanate (FITC) or phycoerythrin (PE), but the tag may also be a peptide sequence e.g., chemically or recombinantly coupled to the polypeptide part of the tagged polypeptide. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

A “signal peptide” may be incorporated and refers to a peptide sequence that directs the transport and localization of the protein within a cell, e.g., to a certain cell organelle (such as the endoplasmic reticulum) and/or the cell surface.

Generally, an “antigen binding domain” refers to the region of the immune receptor, e.g., CAR that specifically binds to an antigen, e.g., to a tumor associated antigen (TAA) or tumor specific antigen (TSA) or the tag of a tagged polypeptide. The immune receptor e.g., CARs of the disclosure may comprise one or more antigen binding domains (e.g., a tandem CAR). Generally, the targeting regions on the CAR are extracellular. The antigen binding domain may comprise an antibody or an antigen binding fragment thereof. The antigen binding domain may comprise, for example, immunoglobulin full length heavy and/or light chains, Fab fragments, single chain Fv (scFv) fragments, divalent single chain antibodies, diabodies, single variable new antigen receptor domain antibody fragments (V-NARs) or heavy-chain antibodies found in camelids (VhH). Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be, for example, the “(G4/S)₃-linker.”

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

There may be a “Spacer” or “hinge,” referring to the hydrophilic region that is between the antigen binding domain and the transmembrane domain. The CARs of the disclosure may comprise an extracellular spacer domain but is it also possible to leave out such a spacer. The spacer may include e.g., Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the immune receptor, e.g., CAR may be derived from any desired natural or synthetic source for such domain. When the source is natural, the domain may be derived from any membrane-bound or transmembrane protein. The transmembrane domain may be derived, for example, from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g., WO2014127261A1) due to small molecule-dependent heterodimerizing domains in each polypeptide of the CAR.

The cytoplasmic signaling domain (or the intracellular signaling domain) of the CAR is responsible for activation of at least one of the normal effector functions of the immune cell in which the CAR is expressed. “Effector function” means a specialized function of a cell, e.g., in a T cell an effector function may be cytolytic activity or helper activity including the secretion of cytokines. The intracellular signaling domain refers to the part of a protein that transduces the effector function signal and directs the cell expressing the CAR to perform a specialized function. The intracellular signaling domain may include any complete, mutated or truncated part of the intracellular signaling domain of a given protein sufficient to transduce a signal that initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRzeta (CD3zeta), FcRgamma, FcRbeta, CD3gamma, CD3delta, CD3epsilon, CD5, CD22, CD79a, CD79b, and CD66d. Most prominent is sequence derived from CD3zeta.

The cytoplasmic domain of the CAR may be designed to comprise the CD3zeta signaling domain by itself or combined with any other desired cytoplasmic domain(s). The cytoplasmic domain of the CAR can comprise a CD3zeta chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3zeta and the signaling domain of CD28. In another example, the cytoplasmic domain may comprise the signaling domain of CD3zeta and the signaling domain of CD137. In a further example, the cytoplasmic domain may comprise the signaling domain of CD3zeta, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD).

In some embodiments, the endodomain may contain a primary cytoplasmic signaling domain or a co-stimulatory region, but not both. In these embodiments, an immune effector cell containing the disclosed CAR is only activated if another CAR containing the missing domain also binds its respective antigen.

In some embodiment of the disclosure, the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g., to an scFv region to render the SUPRA CAR T cell tumor specific. This approach would be particularly useful for generating universal CAR T cells for various tumors; adaptor molecules could be designed for tumor specificity and would provide options for altering specificity post-adoptive transfer, key for situations of selection pressure and antigen escape.

The CARs of the disclosure may be designed to comprise any portion or part of the above-mentioned domains as described herein in any order and/or combination resulting in a functional CAR, i.e., a CAR that mediated an immune effector response of the immune effector cell that expresses the CAR as disclosed herein.

The term “allogeneic” as used herein refers to any material derived from a different subject of the same species as the subject to who the material is re-introduced.

The embodiments of the disclosure may be “isolated,” which means altered or removed from the natural state. For example, an isolated population of cells means an enrichment of such cells and separation from other cells that are normally associated in their naturally occurring state with the isolated cells. An isolated population of cells means a population of substantially purified cells that are a homogenous population of cells.

The terms “specifically binds” or “specific for” with respect to an antigen-binding domain of an antibody, of a fragment thereof or of a CAR, refer to an antigen-binding domain that recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

The terms “engineered cell” and “genetically modified cell” as used herein can be used interchangeably. The terms mean containing and/or expressing a foreign gene or nucleic acid sequence that, in turn, modifies the genotype or phenotype of the cell or its progeny. Especially, the terms refer to the fact that cells, preferentially T cells or NK cells can be manipulated by recombinant methods well known in the art to express stably or transiently peptides or proteins that are not expressed in these cells in the natural state. For example, T cells or NK cells, preferentially human T cells or NK cells are engineered to express an artificial construct such as a chimeric antigen receptor on their cell surface. For example, the CAR sequences may be delivered into cells using a retroviral or lentiviral vector.

The term “tagged polypeptide” as used herein refers to a polypeptide that has bound thereto directly or indirectly at least one additional component, i.e., the tag. The polypeptide may be an antibody or antigen binding fragment thereof that binds to an antigen expressed on the surface of a target cell such as a tumor associated antigen on a cancer cell. The tag may be a hapten such as FITC, biotin, PE, or streptavidin and the hapten may be bound by the anti-hapten (anti-tag) binding domain of the CAR or TCR.

Haptens are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

Alternatively, the tag may also be a peptide sequence e.g., chemically or recombinantly coupled to the polypeptide part of the tagged polypeptide. Tags for “anti-tagCAR systems” are well known in the art and any tag suitable for such a system of anti-tagCAR and tagged polypeptide may be used herein.

Sequence Listing
SEQ ID NO: 1 Light chain variable region of humanized anti-MuTCRβ
antibody:
YELIQPSSASVTVGETVKITCSGDQLPKNFAYWFQQKSDKNILLLIYMDNKRPSGIPERFSGS
TSGTTATLTISGAQPEDEAAYYCLSSYGDNNDLVFGSGTQLTVL
SEQ ID NO: 2 Heavy chain variable region of humanized anti-MuTCRβ
antibody:
EVYLVESGGDLVQPGSSLKVSCAASGFTFSDFWMYWVRQAPGKGLEWVGRIKNKPNNYAT
EYADSVRGRFTISRDDSRNSIYLQMNRLRVDDTAIYYCTRAGRFDHFDYWGQGTMVTVSS
SEQ ID NO: 3 TCR α-chain constant domain:
IQNPDPAVYQLRDSKSSDKSVCLFTDFDSQTNVSQSKDSDVYITDKCVLDMRSMDFKSNS
AVAWSNKSDFACANAFNNSIIPEDTFFPSPESSCDVKLVEKSFETDTNLNFQNLS
SEQ ID NO: 4 TCR δ-chain transmembrane domain:
LGLRMLFAKTVAVNFLLTAKLFF
SEQ ID NO: 5 TCR β-chain constant domain:
EDLKNVFPPKVAVFEPSEAEISHTQKATLVCLATGFYPDHVELSWWVNGKEVHSGVCTDPQ
PLKEQPALNDSRYCLSSRLRVSATFWQNPRNHFRCQVQFYGLSENDEWTQDRAKPVTQIV
SAEAWGRADCGFTSESYQQGVLSATILYEILLGKATLYAVLVSALVLMAMVKRKDSRG
SEQ ID NO: 6 TCR γ-chain transmembrane domain:
YYMYLLLLLKSVVYFAIITCCLL
SEQ ID NO: 7: Human TCRβ chain Domain 3
QNPRNHFRCQVQFYGLSENDEWTQDRAKPVTQIV
SEQ ID NO: 8: Murine TCRβ chain Domain 3 (differences with SEQ
ID NO: 7 underlined)
HNPRNHFRCQVQFHGLSEEDKWPEGSPKPVTQNI
SEQ ID NO: 9 Human TCRβ chain comprising Domain 3 with 2 murinized
amino acid residues (differences with SEQ ID NO: 7 underlined)
EDLKNVFPPKVAVFEPSEAEISHTQKATLVCLATGFYPDHVELSWWVNGKEVHSGVSTDPQ
PLKEQPALNDSRYCLSSRLRVSATFWQNPRNHFRCQVQFYGLSENDEWPQGRAKPVTQIV
SAEAWGRADCGFTSESYQQGVLSATILYEILLGKATLYAVLVSALVLMAMVKRKDSRG
SEQ ID NO: 10: Human TCRβ chain comprising Domain 3 with 9
murinized amino acid residues (differences with SEQ ID NO: 7
underlined; differences with SEQ ID NO: 8 in bold)
EDLKNVFPPKVAVFEPSEAEISHTQKATLVCLATGFYPDHVELSWWVNGKEVHSGVSTDPQ
PLKEQPALNDSRYCLSSRLRVSATFWHNPRNHFRCQVQFHGLSENDKWPEGSAKPVTQNI
SAEAWGRADCGFTSESYQQGVLSATILYEILLGKATLYAVLVSALVLMAMVKRKDSRG
SEQ ID NO: 11: Complete CAR sequence:
MALPVTALL LPLALLLHAA RPYELIQPSS ASVTVGETVK ITCSGDQLPK NFAYWFQQKS
DKNILLLIYM DNKRPSGIPE RFSGSTSGTT ATLTISGAQP EDEAAYYCLS
SYGDNNDLVF GSGTQLTVLG GGGSGGGGSG GGGSEVYLVE SGGDLVQPGS
SLKVSCAASG FTFSDFWMYW VRQAPGKGLE WVGRIKNKPN NYATEYADSV
RGRFTISRDD SRNSIYLQMN RLRVDDTAIY YCTRAGRFDH FDYWGQGTMV
TVSSTTTPAP RPPTPAPTIAS QPLSLRPEAC RPAAGGAVHT RGLDFACDIY
IWAPLAGTCG VLLLSLVITLYCKRGRKKLL YIFKQPFMRP VQTTQEEDGC SCRFPEEEEG
GCELRVKFSR SADAPAYKQG QNQLYNELNL GRREEYDVLD KRRGRDPEMG
GKPRRKNPQE GLYNELQKDK MAEAYSEIGM KGERRRGKGH DGLYQGLSTA
TKDTYDALHM QALPPR

SEQ ID NO:12-15 lists amino acid sequences of TCRs. The variable region is not underlined. The italics sequence in the variable regions corresponds to the CDR3 region. Constant regions of the chains are listed underlined.

SEQ ID NO: 12: Human TCRα chain (clone RA14):
MEKNPLAAPLLILWTHLDCVSILNVEQSPQSLHVQEGDSTNFTCSFPSSNFYALHWYRWETA
KSPEALFVMTLNGDEKKKGRISATLNTKEGYSYLYIKGSQPEDSATYLCARNTGNQFYFGTG
TSLTVIPNIQNPDPAVYQLRDSKSSDKSVCLFTDFDSQTNVSQSKDSDVYITDKCVLDMRSM
DFKSNSAVAWSNKSDFACANAFNNSIIPEDTFFPSPESSCDVKLVEKSFETDTNLNFQNLSVI
GFRILLLKVAGFNLLMTLRLWSS
SEQ ID NO: 13: Human TCRβ chain (clone RA14):
MGIGLLCCAALSLLWAGPVNAGVTQTPKFQVLKTGQSMTLQCAQDMNHEYMSWYRQDPG
MGLRLIHYSVGAGITDQGEVPNGYNVSRSTTEDFPLRLLSAAPSQTSVYFCASSPVTGGIYG
YTFGSGTRLTVVEDLKNVFPPEVAVFEPSEAEISHTQKATLVCLATGFYPDHVELSWWVNGK
EVHSGVCTDPQPLKEQPALNDSRYCLSSRLRVSATFWQNPRNHFRCQVQFYGLSENDEWT
QDRAKPVTQIVSAEAWGRADCGFTSESYQQGVLSATILYEILLGKATLYAVLVSALVLMAMVK
RKDSRG
SEQ ID NO: 14 Human TCRγ chain (clone G115):
MVSLLHASTLAVLGALCVYGAGHLEQPQISSTKTLSKTARLECVVSGITISATSVYWYRERPG
EVIQFLVSISYDGTVRKESGIPSGKFEVDRIPETSTSTLTIHNVEKQDIATYYCALWEAQQELG
KKIKVFGPGTKLIITDKQLDADVSPKPTIFLPSIAETKLQKAGTYLCLLEKFFPDVIKIHWEEK
KSNTILGSQEGNTMKTNDTYMKFSWLTVPEKSLDKEHRCIVRHENNKNGVDQEIIFPPIKTDVI
TMDPKDNCSKDANDTLLLQLTNTSAYYMYLLLLLKSVVYFAIITCCLLRRTAFCCNGEKS
SEQ ID NO: 15 Human TCRδ chain (clone G115):
MERISSLIHLSLFWAGVMSAIELVPEHQTVPVSIGVPATLRCSMKGEAIGNYYINWYRKTQGNT
MTFIYREKDIYGPGFKDNFQGDIDIAKNLAVLKILAPSERDEGSYYCACDTLGMGGEYTDKLI
FGKGTRVTVEPRSQPHTKPSVFVMKNGTNVACLVKEFYPKDIRINLVSSKKITEFDPAIVISPS
GKYNAVKLGKYEDSNSVTCSVQHDNKTVHSTDFEVKTDSTDHVKPKETENTKQPSKSCHKP
KAIVHTEKVNMMSLTVLGLRMLFAKTVAVNFLLTAKLFFL

Listed below are (parts of) sequences as available from the Genbank database from NCBI under numbers AAT27465, AAK49780, AAT27464, X02883, M64239, M12887, M12888, X02384(AH002088), M26057, M22148, M23381, M14996, M15002, M17323, M13340, M12834, M12837, AF021335.

TABLE 1
Amino acid sequences of (parts of) T cell receptors of human
or mouse origin.

| SEQ | | |
|---|---|---|
| ID | | |
| NO. | Description | Sequence |
| 16 | AAT27465 306 aa T-cell receptor beta chain precursor Mus musculus | matrllcytv lcllgariln skviqtpryl vkgqgqkakm rcipekghpv vfwyqqnknn efkflinfqn qevlqqidmt ekrfsaecps nspcsleiqs seagdsalyl casslsgggt evffgkgtrl tvvedlrnvt ppkvslfeps kaeiankqka tlvclargff pdhvelswwv ngkevhsgvs tdpqaykesn ysyclssrlr vsatfwhnpr nhfrcqvqfh glseedkwpe gspkpvtqni saeawgradc gitsasyhqg vlsatilyei llgkatlyav lvsglvlmam vkkkns |
| 17 | AAK49780 306 aa T-cell receptor beta chain precursor Mus musculus | mnkwvfcwvt lclltvetth gdggiitqtp kfligqegqk ltlkcqqnfn hdtmywyrqd sgkglrliyy sitendlqkg dlsegydasr ekkssfsltv tsaqknemav flcasgdwgy eqyfgpgtrl tvledlrnvt ppkvslfeps kaeiankqka tlvclargff pdhvelswwv ngkevhsgvs tdpqaykesn ysyclssrlr vsatfwhnpr nhfrcqvqfh glseedkwpe gspkpvtqni saeawgradc gitsasyhqg vlsatilyei llgkatlyav lvsglvlmam vkkkns |
| 18 | AAT27464 269 aa T-cell receptor alpha chain precursor Mus musculus | mvlallpvlg ihfvlrdaqa qsvtqpdarv tvsegaslql rckysysgtp ylfwyvqypr qglqlllkyy sgdpvvqgvn gfeaefsksn ssfhlrkasv hwsdsavyfc vlsedsnyql iwgsgtklii kpdiqnpepa vyqlkdprsq dstlclftdf dsqinvpktm esgtfitdkt vldmkamdsk sngaiawsnq tsftcqdifk etnatypssd vpcdatltek sfetdmnlnf qnlsvmglri lllkvagfnl lmtlrlwss |
| 19 | AAK49779 268 aa T-cell receptor alpha chain precursor Mus musculus | mkrllcsllg llctqvcwvk gqqvqqspas lvlqegenae lqcnfsstat rlqwfyqrpg gslvsllynp sgtkhtgrlt sttvtkerrs slhisssqtt dsgtyfcats svntgnykyv fgagtrlkvi ahiqnpepav yqlkdprsqd stlclftdfd sqinvpktme sgtfitdktv ldmkamdsks ngaiawsnqt sftcqdifke tnatypssdv pcdatlteks fetdmnlnfq nlsvmglril llkvagfnll mtlrlwss |
| 20 | X02883 | DIQNPDPAVYQLRDSKSSDKSVCLFTDFDSQTNVSQSKDSDVYI TDKTVLDMRSMDFKSNSAVAWSNKSDFACANAFNNSIIPEDTFFPSPESSCDVKLV EK SFETDTNLNFQNLSVIGFRILLLKVAGFNLLMTLRLWSS |
| 21 | M64239 | IQNPEPAVYQLKDPRSQDSTLCLFTDFDSQINVPKTMESGTFITDKTVL DMKAMDSKSNGAIAWSNQTSFTC |
| 22 | M12887 | DLNKVFPPEVAVFEPSEAEISHTQKATLVCLATGFFPDHVELSW WVNGKEVHSGVSTDPQPLKEQPALNDSRYCLSSRLRVSATFWQNPRNHFRCQVQFY GL SENDEWTQDRAKPVTQIVSAEAWGRADCGFTSVSYQQGVLSATILYEILLGKATLY AV LVSALVLMAMVKRKDF |
| 23 | M12888 | DLKNVFPPEVAVFEPSEAEISHTQKATLVCLATGFYPDHVELSW WVNGKEVHSGVSTDPQPLKEQPALNDSRYCLSSRLRVSATFWQNPRNHFRCQVQFY GL SENDEWTQDRAKPVTQIVSAEAWGRADCGFTSESYQQGVLSATILYEILLGKATLY AV LVSALVLMAMVKRKDSRG |
| 24 | AH002088 (X02384) | DLRNVTPPKVSLFEPSKAEIANKQKATLVCLARGFFPDHVELSW WVNGKEVHSGVSTDPQAYKESNYSYCLSSRLRVSATFWHNPRNHFRCQVQFHGLSE ED KWPEGSPKPVTQNISAEAWGRADCGITSASYQQGVLSATILYEILLGKATLYAVLV ST LVVMAMVKRKNS |
| 25 | M26057 | DLRNVTPPKVSLFEPSKAEIANKQKATLVCLARGFFPDHVELSW WVNGKEVHSGVSTDPQAYKESNYSYCLSSRLRVSATFWHNPRNHFRCQVQFHGLSE ED KWPEGSPKPVTQNISAEAWGRADCGITSASYHQGVLSATILYEILLGKATLYAVLV SG LVLMAMVKKKNS |
| 26 | M22148 | PSYTGGYADKLIFGKGTRVTVEPRSQPHTKPSVFVMKNGTNVAC LVKEFYPKDIRINLVSSKKITEFDPAIVISPSGKYNAVKLGKYEDSNSVTCSVQHD NK TVHSTDFEVKTDSTDHVKPKETENTKQPSKSCHKPKAIVHTEKVNMMSLTVLGLRM LF AKTVAVNFLLTAKLFFL |
| 27 | M23381 | SQPPAKPSVFIMKNGTNVACLVKDFYPKEVTISLRSSKKIVEFD PAIVISPSGKYSAVKLGQYGDSNSVTCSVQHNSETVHSTDFEPYANSFNNEKLPEP EN DTQISEPCYGPRVTVHTEKVNMMSLTVLGLRLLFAKTIAINFLLTVKLFF |
| 28 | M14996 | KQLDADVSPKPTIFLPSIAETKLQKAGTYLCLLEKFFPDVIKIH WQEKKSNTILGSQEGNTMKTNDTYMKFSWLTVPEKSLDKEHRCIVRHENNKNGVDQ EI IFPPIKTDVITMDPKDNCSKDANDTLLLQLTNTSAYYMYLLLLLKSVVYFAIITCC LL RRTAFCCNGEKS |
| 29 | M15002 | KQLDADVSPKPTIFLPSIAETKLQKAGTYLCLLEKFFPDIIKIH WQEKKSNTILGSQEGNTMKTNDTYMKFSWLTVPEESLDKEHRCIVR HENNKNGIDQEIIFPPIKT |
| 30 | M17323 | KQLDADVSPKPTIFLPSIAETKLQKAGTYLCLLEKFFPDIIKIH WQEKKSNTILGSQEGNTMKTNDTYMKFSWLTVPEESLDKEHRCIVRHENNKNGIDQ EI IFPPIKTDVTTVDPKDSYSKDANDVTTVDPKYNYSKDANDVITMDPKDNWSKDAND TL LLQLTNTSAYYMYLLLLLKSVVYFAIITCCLLGRTAFCCNGEKS |
| 31 | M13340 | KRLDADISPKPTIFLPSVAETNLHKTGTYLCLLEKFFPDVIRVY WKEKDGNTILDSQEGDTLKTNDTYMKFSWLTVPERAMGKEHRCIVKHENNKGGADQ EI FFPSIKKVAVSTKPTTCWQDKNDVLQLQFTITSAYYTYLLLLLKSVIYLAIISFSL LR RTSVCGNEKKS |
| 32 | M12834 | KRLDADISPKPTIFLPSVAETNLHKTGTYLCLLEKFFPDVIRVY WKEKNGNTILDSQEGDTLKTKGTYMKFSWLTVPERAMGKEHSCIVKHENNKGGADQ EI FFPSIKKVATTCWQDKNDVLQFQFTSTSAYYTYLLLLLKSVIYLAIISFSLLRRTS VC GNEKKS |
| 33 | M12837 | KKLDADISPKPTIFLPSVAETNLHKTGTYLCVLEKFFPDVIRVY WKEKKGNTILDSQEGDMLKTNDTYMKFSWLTVPERSMGKEHRCIVKHENNKGGADQ E IFFPTIKKVAVSTKPTTCWQDKNDVLQLQFTITSAYYTYLLLLLKSVIYLAIISFS LLR RTSVCCNEKKS |
| 34 | AF021335 | KRLDADISPKPTIFLPSVAETNLHKTGTYLCLLEKFFPDVIRVY WKEKNGNTILDSQEGDTLKTKGTYMKFSWLTVPERAMGKEHSCIVKHENNKGGADQ EI FFPSIKKVATTCWQDKNDVLQFQFTSTSAYYTYLLLLLKSVIYLAIISFSLLRRTS VC GNEKKS |

SEQ ID NO: 35 Human TCRβ chain Domain 3 with 2
murinized amino acid residues (differences with
SEQ ID NO: 7 underlined)
QNPRNHFRCQVQFYGLSENDEWPQGRAKPVTQIV
SEQ ID NO: 36: Human TCRβ chain Domain 3 with 9
murinized amino acid residues (differences with
SEQ ID NO: 7 underlined; differences with SEQ ID
NO: 8 in bold)
HNPRNHFRCQVQFHGLSENDKWPEGSAKPVTQNI
SEQ ID NO: 37:
EVQLVESGGGLVQPGGSLRLSCAASGFTFSNYAMSWVRQAPGKGLEWVATI
TGGGSYTYYPDSVKGRFTISRDNSKNTLYLQMNSLRAEDTAVYYCVRQRVG
DYVSSLLGYWGQGTLVTVSSGGGGSGGGGSGGGGSDVVMTQSPLSLPVTLG
QPASISCRSSQSLVHSNGNTYLHWFLQRPGQSPRLLIYKVSNRFSGVPDRF
SGSGSGTDFTLKISRVEAEDVGVYYCSQSTHVPYTFGQGTKLEIK

Experimental Part

### EXAMPLE 1

T cell engineering strategies, which incorporate a method for the purification of genetically modified T cells, as well as engineered T cell deletion after transfer into patients, are needed to increase efficacy, reduce potential side effects, and improve safety. By characterizing the antigen binding site of a GMP-grade anti-αβTCR antibody, usually used for clinical grade depletion of αβT cells from stem cell transplantation products, a strategy was developed that allows for the negative selection, i.e., untouched purification of αβTCR engineered immune cells by changing specifically two amino acids in the TCR β chain constant domain of introduced exogenous TCR chains. Vice versa, a humanized anti-αβTCR antibody was engineered that targets an extended mutated region of specifically nine amino acids in the exogenous TCR chain constant domain, in order to allow for later depletion of engineered immune cells. This strategy can be applied to any T cell engineering strategy that interferes with the endogenous αβTCR chains.

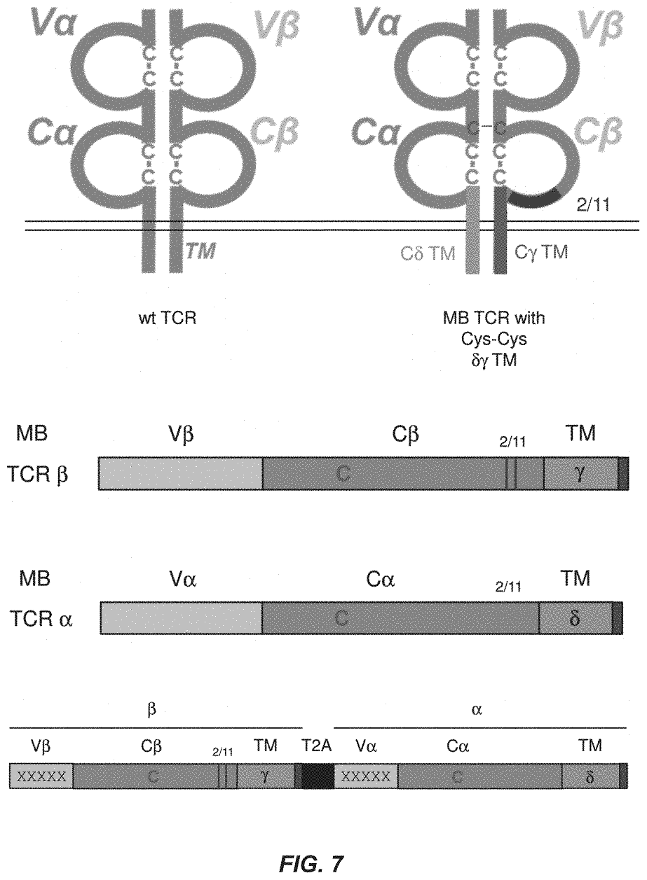

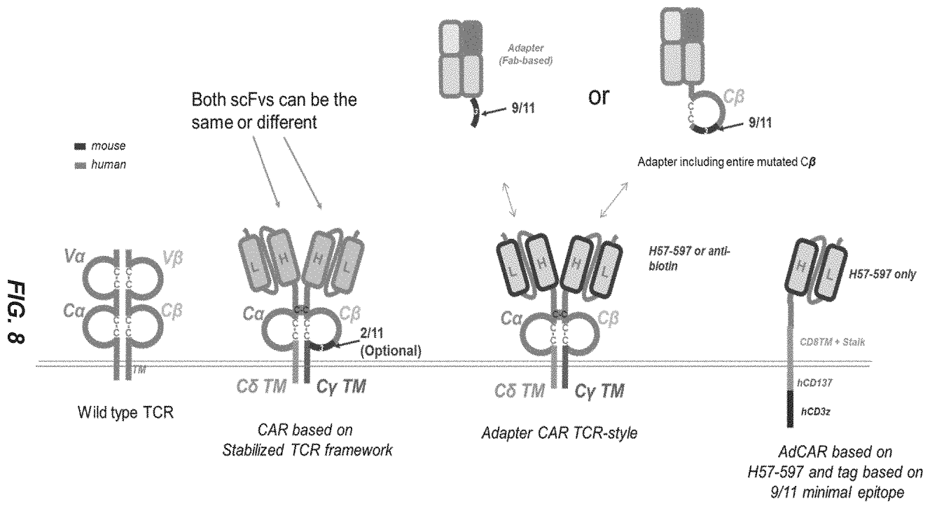

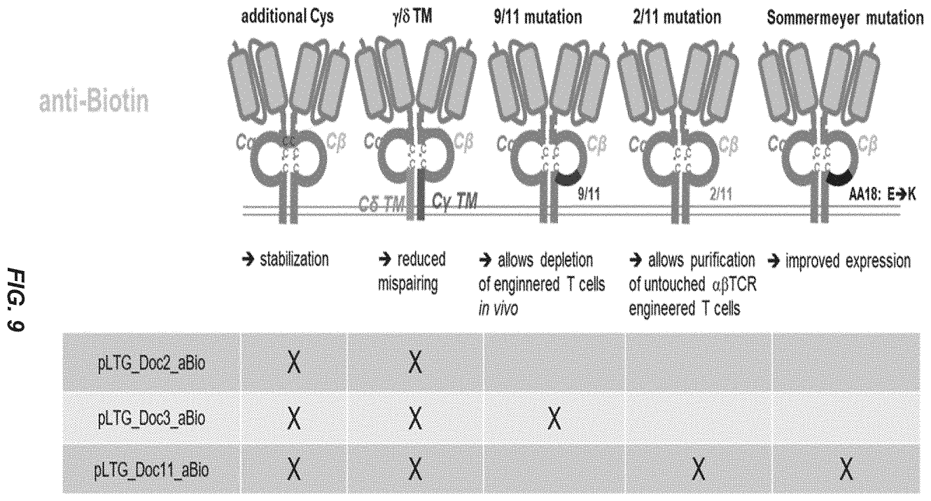

The expression of the exogenous immune receptors on the engineered immune cells was able to be enhanced by means of replacing the α-chain transmembrane domain and/or the β-chain transmembrane domain of the human αβTCR by the δ-chain transmembrane domain and/or the γ-chain transmembrane domain of the human γδTCR counterpart. Also, the optional introduction of an additional Cys bridge was found to lead to enhanced expression and avoid mispairing with endogenous TCR chains. Accordingly, an extracellular and transmembrane frame is provided that not only competes with the endogenous TCR, but also allows formation of a complex with CD3 that will allow cytoplasmic signaling. See e.g., FIGS. 7 and 8.

Additionally, it was found that the specifically murinized residues as referred to above improve expression of the exogenous immune receptor even further.

Finally, it was considered that the combination of the (humanized) anti-αβTCR antibody and its binding epitope, i.e., the extended mutated region of specifically nine amino acids in the exogenous TCR β chain constant domain, allows for the use of an adapter concept, wherein an immune cell expressing an immune receptor comprising the antigen binding domain of the anti-αβTCR antibody is combined with a polypeptide that can specifically bind a target, for example, a cancer cell, and wherein the polypeptide comprises the extended mutated region of specifically nine amino acids in the exogenous TCR β chain constant domain, such that it can be recognized by the immune cell. See e.g., FIG. 8.

Results

Anti-Human αβTCR Binds an Epitope on the TCRβ Chain of Human αβT Cells

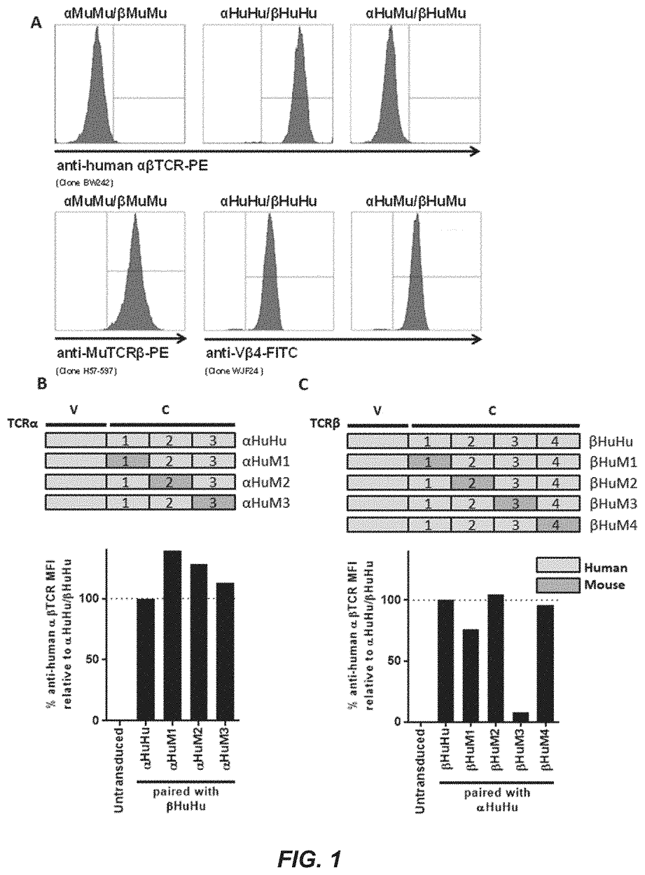

The GMP-grade anti-human αβT cell receptor (TCR) monoclonal antibody clone BW242/412 (from now on referred to as anti-human aαβTCR) recognizes a common determinant of the human TCRα/β-CD3 complex, which has not been characterized yet. In order to allow further epitope mapping, the antibody's ability to bind to murine αβTCRs was first tested. Therefore, Jurma T cells, a TCR-deficient T cell line, were transduced with human αβTCRs directed against the cancer/testis antigen NY-ESO-1₁₅₇₋₁₆₅ (23) or with a murine nonsense αβTCR composed of the TCRα chain of an MDM2-specific αβTCR (24) and the TCRβ chain of a p53-specific αβTCR (25). Specific binding of anti-human αβTCR was only observed to the human (αHuHu/βHuHu) but not the murine (αMuMu/βMuMu) TCR transduced Jurma cells (FIG. 1A). To exclude that parts of the human variable domain of the used anti-human αβTCR are involved in binding, the human NY-ESO-1 αβTCR variable domain was grafted on the murine constant domain to create a chimeric αβTCR (αHuMu/βHuMu). Replacing only the human TCRα and TCRβ constant domains by murine equivalents completely abrogated binding of anti-human αβTCR, to levels resembling binding to a fully murine αβTCR (αMuMu/βMuMu). This indicates that the human constant domain contains the binding epitope. Comparable transgenic expression of murine and human TCRs was confirmed by anti-MuTCRβ and anti-Vβ4 respectively (FIG. 1A). Infusion of T cells expressing TCRs with complete murine constant domains into patients can generate immunogenic effects and lead to a decreased persistence of the engineered cells in vivo (26). To minimize these undesirable effects, it was aimed to map the minimal amount of murine residues needed to disrupt binding of anti-human αβTCR, by making use of previously described chimeric-TCRα and (3 chains, with mutational blocks covering all amino acid differences between the constant regions of human and mouse αβTCRs (23). Three NY-ESO-1 TCRα chain variants and four NY-ESO-1 TCRβ chain variants were tested, each containing one murine domain flanked by complete human amino acid sequences. Every TCRα chain was paired with the fully human TCRβ chain (βHuHu) (FIG. 1B) and every TCRβ chain was paired with the fully human TCRα chain (αHuHu) (FIG. 1C) and introduced into Jurma cells, after which binding of anti-human αβTCR was determined by flow cytometry. Transduction efficiency of the constructs was measured by anti-Vβ4 and was comparable in all conditions. Antibody binding was significantly impaired in T cells expressing the αβTCR that includes murine domain 3 (βHuM3), while none of the other chimeric αβTCRs substantially impaired anti-human αβTCR binding (FIGS. 1B and C). These results indicate that domain 3 of the TCRβ chain (αβHuM3) dictates the binding of anti-human αβTCR.

Anti-Human αβTCR Binding Can be Abrogated by Mutating 2 Residues

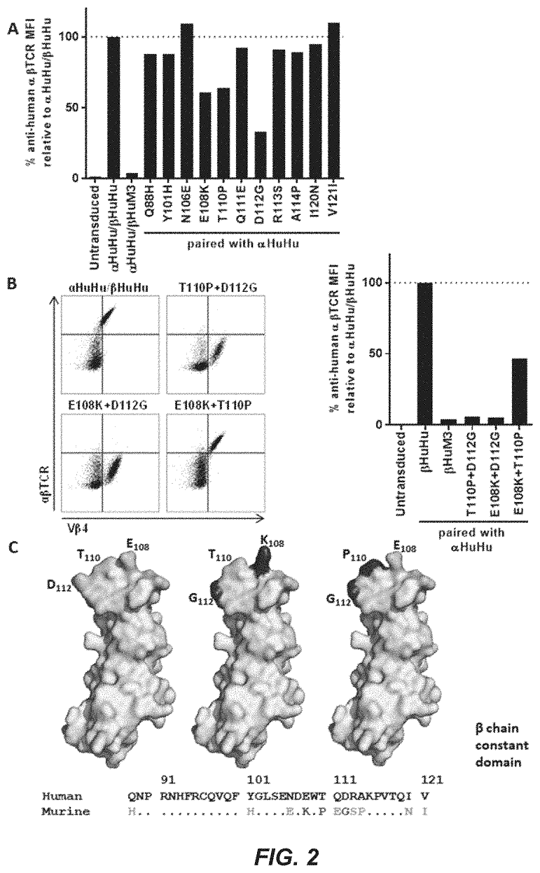

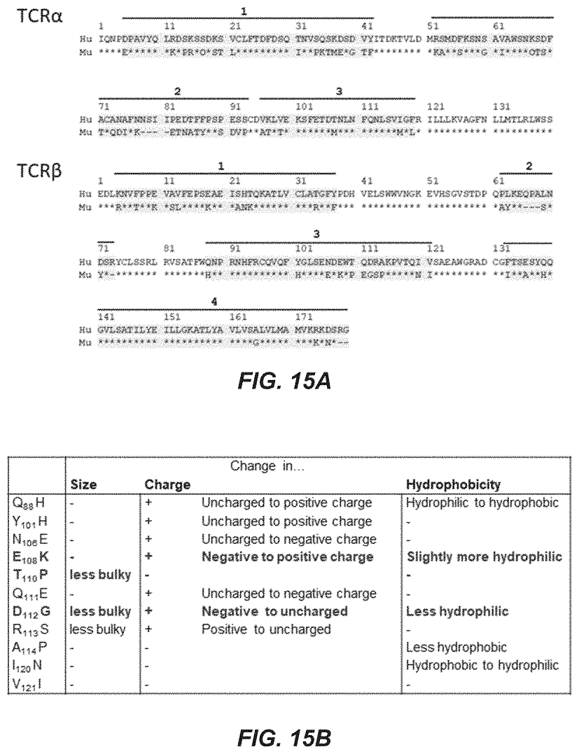

Analysis of the sequence of domain 3 of the TCRβ chain constant domain revealed eleven residues that are non-homologous between murine and human species (FIG. 16C). To determine which residues are essential for anti-human αβTCR binding, eleven variants of the TCRβ chain were constructed in which each one of the non-homologous amino acids was replaced by the murine counterpart. These eleven constructs were paired with the completely human αTCR chain (αHuHu), introduced in Jurma cells and tested for binding by the anti-human αβTCR antibody. Of the eleven generated mutants, the substitutions of ‘human’ glutamic acid (E108) to the ‘murine’ lysine (K), ‘human’ threonine (T110) to the ‘murine’ proline (P), and ‘human’ aspartic acid (D112) to the ‘murine’ glycine (G) showed a substantial abrogation of anti-human αβTCR binding (FIG. 2A). However, none of these substitutions was sufficient to induce total abrogation as shown by the TCR consisting of αHuHu/βHuM3. Therefore, TCRβ chains were constructed with a combination of the aforementioned mutations. The TCRβ chains with a D112G mutation combined with E108K or T110P were both effective in abrogating binding of the anti-human αβTCR antibody (FIG. 2B), which can be explained by a substantial decrease in bulkiness, thus a decrease in size of these residues (FIG. 2C and FIG. 15B). For further engineered T cell experiments, the combination of T110P and D112G murinization was selected.

Purification of αβTCR Engineered T Cells Using Anti-Human αβTCR MACS

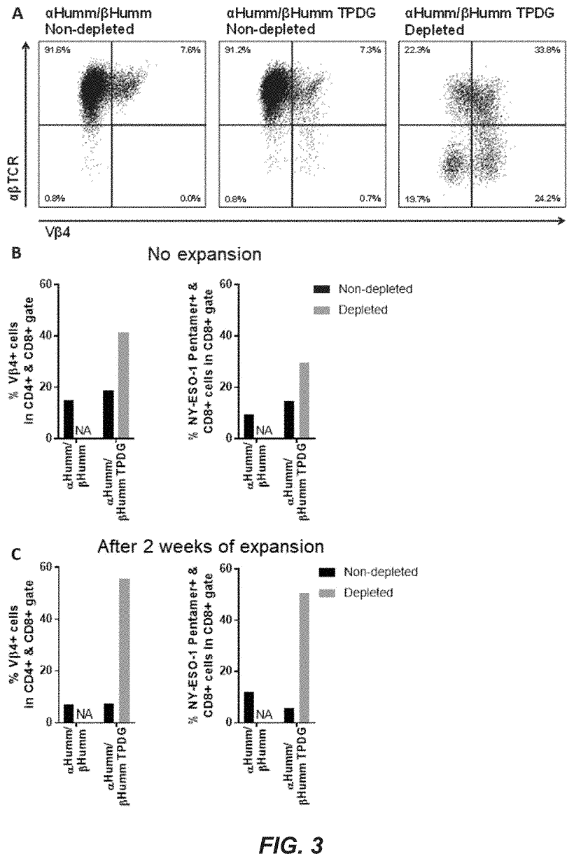

Due to the competition of introduced αβTCR chains with endogenous αβTCR chains in primary T cells, the introduction of foreign αβTCRs is frequently impaired when compared to αβTCR deficient Jurma cells. Murine αβTCRs, or residues derived from murine αβTCRs introduced into human αβTCRs, and expressed in human T cells, have been reported to outcompete endogenous human TCR chains (27-29). Furthermore, these murine and murinized αβTCRs preferentially pair with each other, thereby decreasing the occurrence of mispairing with endogenous human αβTCRs. Therefore, single murine amino acids were utilized to enhance the expression of introduced TCRs. These “minimally murinized” constant domain variants (from now on referred to as mm) contain murine amino acids that are both critical and sufficient to improve pairing between the two chains. Next, the above-identified murine residues (T110P+D112G) were introduced in the TCRβ chain constant domain in order to test whether this indeed was sufficient to disrupt the binding of anti-human αβTCR in human primary T cells. To test this concept, healthy donor T cells were transduced with mm NY-ESO-1 specific αβTCRs as a negative control or mm NY-ESO-1 specific αβTCRs, including the two identified mutations T110P+D112G. The αβTCR and Vβ4 expression after transduction were assessed by flow cytometry. The fraction of cells positive for anti-Vβ4, but negative for anti-human αβTCR is the fraction of interest (FIG. 3A, middle plot). Magnetic-activated cell sorting (MACS) depletion using anti-human αβTCR resulted in a significant increase of the Vβ4 engineered T cell fraction that was still visible after 2 weeks of expansion (FIG. 3A, right plot, right quadrants). The T cell fraction present in the upper left quadrant are αβTCR positive and Vβ4 negative, likely due to the re-expression of the endogenous TCR. All surviving residual non-T cells that are still present at the moment of MACS depletion are not removed by this method and therefore visible in the lower left quadrant. A comparison of purity directly after isolation, and after two weeks of expansion, and investigation of not only the introduced Vβ4 chain but also of pairing, thus the specificity of introduced chains by anti-Vβ4 and NY-ESO-1₁₅₇₋₁₆₅ HLA*02:01 pentamer staining, demonstrated that the introduced new mutations do not interfere with the used mm-pairing strategy (FIG. 3B) and that purity of the engineered cells is maintained two weeks after expansion (FIG. 3C).

Enrichment Strategy Within the Context of Alternative Stabilization Procedures

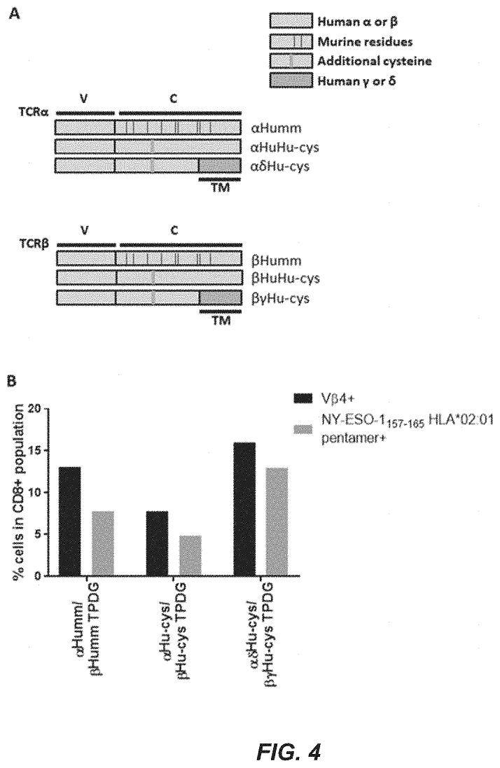

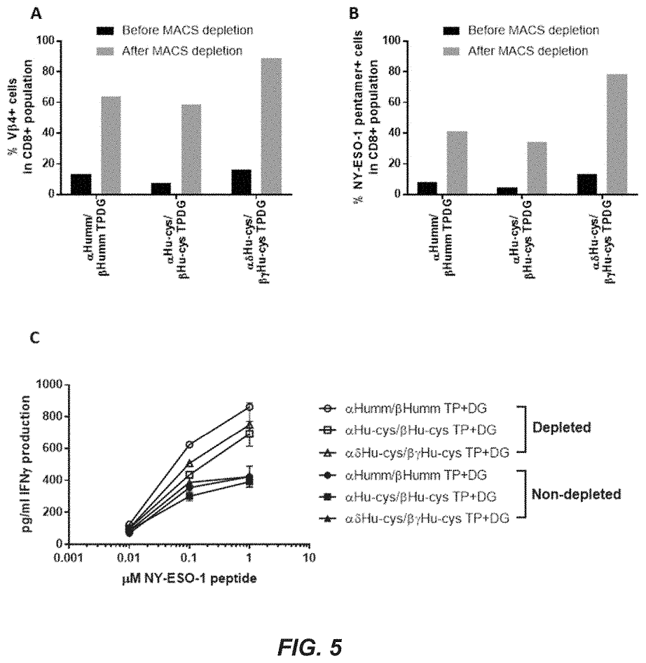

Multiple alternative strategies to prevent αβTCR chain mispairing and thereby increasing the expression of the introduced tumor specific αβTCR have been reported. E.g., adding an additional cysteine residue, to introduce a disulfide bridge between the α and β chains, has been shown to increase expression and decrease mispairing (30). Also, human γδTCRs introduced in human T cells do not pair with endogenous αβTCRs (31), therefore it is attractive to use γδTCR constant domains for engineering αβT cells in a similar way. The present enrichment strategy was tested to determine if it could also be combined with these alternative pairing solutions. Firstly, an NY-ESO-1 specific TCR was constructed with an additional disulfide bridge by the mutation of one specific residue in each chain; T48C in TCRCα and S57C in TCRCβ. Secondly, an NY-ESO-1 specific TCR was constructed with the same additional disulfide bridge and with a human γδTCR trans-membrane domain. A schematic representation of all three approaches is displayed in FIG. 4A. To later make use of the αβTCR depletion method, the mutations T110P+D112G were introduced in the β chains, and then assessed the expression of the different TCRs in primary T cells by measuring the percentage of Vβ4+ and NY-ESO-1₁₅₇₋₁₆₅ HLA*02:01 pentamer+ cells within the CD8+ population (FIG. 4B). All three conditions resulted in a NY-ESO-1₁₅₇₋₁₆₅ HLA*02:01 pentamer+ CD8+ fraction almost as big as the Vβ4+ CD8+ fraction, indicating that all TCRs are preferentially paired. The three different conditions were αβTCR depleted in the same way as before, and the percentage of Vβ4+ cells (FIG. 5A) and NY-ESO-1₁₅₇₋₁₆₅ HLA*02:01 pentamer+ cells within the CD8+ population (FIG. 5B) was measured by flow cytometry. All three described methods were suitable for creating preferential pairing and subsequent purification by this αβTCR depletion method. Thus, partial murinization or stabilization through cysteines are equally potent and adding γ and δ domains may enhance expression or pairing.

Augmented In Vitro Tumor Cell Recognition by Purified Engineered T Cells.

To determine if purified NY-ESO-1₁₅₇₋₁₆₅ αβTCR engineered T cells were superior in target cell recognition compared to non-purified cells, T2 cells were pulsed with multiple concentrations of NY-ESO-1₁₅₇₋₁₆₅ peptide. Purified engineered T cells showed a stronger response to the peptide loaded T2 cells than the non-purified cells. However, no substantial differences between the three pairing strategies could be observed (FIG. 5C). In further engineered T cell experiments, the mm approach was used to prevent mispairing and increase expression.

Developing an Antibody Recognizing the Introduced Mutated Region

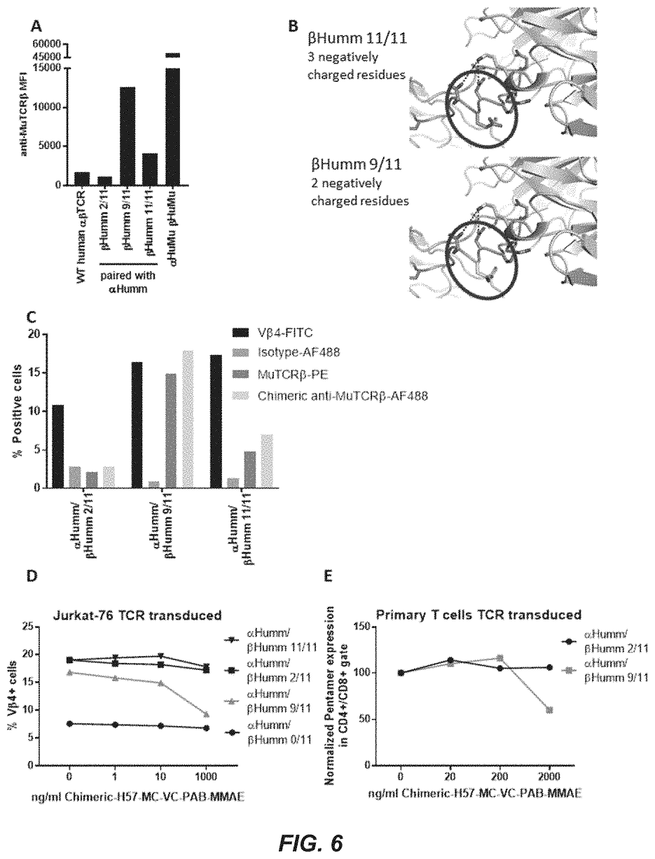

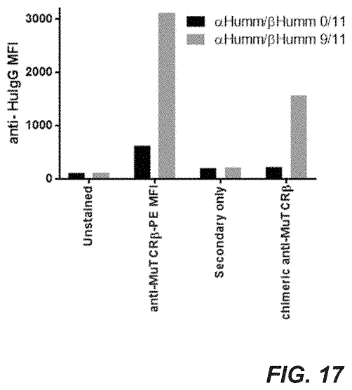

The infusion of engineered T cells can potentially be toxic, due to the occurrence of cytokine release syndrome (13) or off-target toxicity of the receptor used (14). To be able to deplete infused engineered T cells in vivo when deemed necessary, it was aimed to raise an antibody specific for the T110P+D112G murinized variant of the αβTCR by immunizing three Wistar rats with a human-mouse chimeric peptide. Despite the fact that antibodies were formed against the chimeric peptide (FIG. 16A), no antibody binding against surface-expressed αβTCRs could be detected (FIG. 16B). Therefore, the commercially available anti-murine TCRβ chain antibody clone H57-597 (from now on referred to as anti-MuTCRβ) was assessed to determine if it was able to bind the murinized αβTCRs on Jurkat-76 cells generated so far. Jurkat-76 cells expressing the T110P+D112G murinized variant of the αβTCR (indicated by βHumm 2/11; two out of the eleven non-homologous amino acids in the 3^{rd} domain are murinized) were not bound by anti-MuTCRβ, however, Jurkat-76 cells expressing the βHummM3 murinized variant of the αβTCR (indicated by βHumm 11/11; all eleven non-homologous amino acids in the 3^{rd} domain are murinized) were bound by anti-MuTCRβ. To limit the amount of murine amino acids introduced, a variant was constructed in which 9/11 non-homologous amino acids in the 3^{rd} domain are murinized (FIG. 16C). Both 11/11 and 9/11 non-homologous murine amino acids in β chain of domain 3 were sufficient to reestablish binding of anti-MuTCRβ, however, not to the same extent as the complete murine αβTCR (FIG. 6A), while fewer murinized mutants, including the T110P+D112G (2/11) mutations did not allow binding of anti-MuTCRβ. Surprisingly, 9/11 caused a higher MFI than 11/11. Structural analyses suggested that this differential binding could be a consequence of the fact that 9/11 contains one less negatively charged residue and therefore results in a more focused electrostatic potential to attract the lysine on CDR1 of anti-MuTCRβ (FIG. 6B). Since the clone of anti-MuTCRβ antibody is of Armenian Hamster origin and presumably induces severe side effects once administered to humans, like anti-thymocyte globulin (32), it was aimed to generate a humanized variant of anti-MuTCRβ. Therefore chimeric variants of anti-MuTCRβ (H57-597, PDB entry code: 1NFD) were generated by exchanging the hamster IgG2 constant domain for the human IgG1 constant domain (referred to as chimeric anti-MuTCRβ). The binding of this newly constructed antibody in engineered Jurkat-76 cells was tested, which resulted in specific antibody binding to the 9/11 murinized TCRβ chain expressed on Jurkat-76 (FIG. 17). To determine the capacity of the chimeric anti-MuTCRβ antibody to bind to primary T cells expressing the murinized αβTCRs, this antibody and an isotype control was conjugated to Alexa Fluor 488 (AF488) and determined binding by flow cytometry. The chimeric anti-MuTCRβ antibody was able to bind both 9/11 and 11/11 murinized TCRs and, as observed in FIG. 6A, the binding to 9/11 was stronger than to 11/11 (FIG. 6C).

Depletion of Engineered Immune Cells Through a Mutation-Specific Antibody

To assess if the chimeric variant of anti-MuTCRβ was able to selectively deplete engineered T cells in vitro, the antibody was coupled to monomethyl auristatin E (MMAE), a cell cycle inhibitor, using the protease cleavable linker VC-PAB (33), to create an antibody-drug conjugate (ADC). Jurkat-76 cells transduced with different murinized TCRs were incubated with multiple concentrations of the ADC. The highest concentration of chimeric H57-MC-VC-PAB-MMAE led to a decrease of Vβ4 positivity in the 9/11 condition only (FIG. 6D). This specific decrease indicates that the ADC is able to selectively deplete 9/11, and not 11/11 αβTCR engineered Jurkat-76 in vitro, most likely due to the weaker binding of the engineered antibody to the 11/11 αβTCR (FIG. 6C). To assess whether this mechanism is also effective if introduced TCRs need to compete with endogenous TCRs, primary T cells transduced with the 2/11 and 9/11 murinized αβTCRs were αβTCR depleted with the antibody selectively recognizing wild type αβTCR, expanded using this REP protocol and incubated with the ADC for 24 hours. As observed for αβTCR transduced Jurkat-76 cells, the 9/11 murinized αβTCR engineered cells were selectively depleted, as indicated by a substantial decrease in NY-ESO-1₁₅₇₋₁₆₅ HLA*02:01 pentamer positivity (FIG. 6E). Although the concentrations of chimeric H57-MC-VC-PAB-MMAE needed to be effective in vitro are higher than one would expect from an MMAE-ADC, this is potentially irrelevant in vivo due to additional cleavage of the VC-PAB linker by extracellular proteases (34).

Findings

Replacing only two amino acids within the constant domain of the TCR β chain allows for the purification of αβTCR engineered T cells by negative selection with GMP-ready tools, which are currently used in daily clinical practice for purification of hematopoietic transplants from αβT cells (35). The very same region on the TCR β chain can also serve as target for antibodies, which can deplete engineered immune cells. This select-kill mechanism is a novel and unique strategy for increasing purity and augmenting safety of αβTCR engineered T cells with minor engineering steps, after transfer into patients.

A sufficient down-regulation of the endogenous αβTCR chains by the introduced αβTCR chains is preferred for this method. Therefore, strategies interfering with endogenous αβTCRs or utilizing knock out of the α or β locus to enhance expression of introduced αβTCRs (36) will benefit from this strategy. However, engineering of T cells via ZFN, CRISPR or TALENs (37) require additional engineering steps and therefore are an additional hurdle for GMP grade production. Dominance of the introduced receptors was accomplished by using a previously described method where human residues are replaced by key murine counterparts (23). Furthermore, it was successfully assessed whether the introduction of an additional disulfide bridge or the exchange of the human αβTCR transmembrane domain for the human γδTCR counterpart could also lead to enhanced expression. Thus, in line with a recently published solution for TEGs (20), an elegant and minimalistic strategy to purify αβTCR engineered T cells was found. This is particularly important in the light of the current practice that infused engineered products harbor only between 15-55% of engineered immune cells (39, 40). The lack of purity can become a major clinical obstacle in terms of efficacy (20) as well as toxicity (13, 41).

Many tumor-associated tumor antigens targeted by αβTCR gene therapy are not exclusively expressed on tumor cells (42). Thus, depending on the type of the antigen targeted by the introduced αβTCR, depletion strategies can be useful. This is illustrated by multiple clinical trials, which have led to devastating results caused by off-target or on-target but off-tumor toxicities (3, 14). Preclinical strategies to predict off-target toxicities by affinity enhanced TCRs provide an important tool to minimize these risks (43). However, these strategies are not infallible, and therefore it is extremely valuable to be able to deplete engineered immune cells with affinity matured receptors, or when targeting novel antigens or antigens that are also partially expressed on healthy tissues. Methods described so far for introducing a safety switch in engineered T cell products rely on the introduction of additional genes for the expression of (truncated) targetable proteins, the introduction of inducible caspase proteins (44) or sensitivity to ganciclovir in the case of the widely used HSV-TK suicide gene (16). The method described here, using minimal murine amino acid substitutions, is not only suitable for creating an untouched population of purified T cells, but also allows for in vivo depletion when needed. The identified two murine amino acids to enable αβTCR depletion can be expanded with an additional seven, to create a chimeric TCR β chain with a total of nine murine amino acids. The major advantage of this strategy, as compared to strategies using e.g., myc-tags introduced into the TCR α chain (17), is its combined property as a selection and safeguard system, as well as the usage of natural αβTCR domains that do most likely not affect signaling or impair pairing.

In conclusion, the murinization of two specific residues in the TCRβ constant domain allows for the untouched isolation of αβTCR engineered T cell products. When a safeguard of engineered immune cells is required, mutating additional seven human amino acids to murine residues in the TCRβ constant domain allows binding of an antibody, which then selectively recognizes engineered T cells. Ultimately, this chimeric receptor design and subsequent purification can be rapidly implemented in any engineering procedure for TCRs used for targeting hematological or solid malignancies. This will allow for further enhancement, efficacy and reduction of adverse effects caused by non- and poorly-engineered T cells. With the additional safety switch, engineered T cells can be depleted at a later time point.

Materials and Methods

Cells and Cell Lines

Phoenix-Ampho cells (CRL-3213) were obtained from ATCC and cultured in DMEM (Thermo Fisher Scientific, Breda, The Netherlands) containing 1% Pen/Strep (Invitrogen) and 10% FCS (Bodinco, Alkmaar, The Netherlands). The TCRβ−/− Jurma cell line (a derivate of Jurkat J.RT3-T3.5 cells (45)), a kind gift from Erik Hooijberg (VU Medical Center, Amsterdam, The Netherlands), TCRβ−/− Jurkat-76, a kind gift from Edite Antunes (Johannes Gutenberg-University, Mainz, Germany) and the T2 cell line (ATCC CRL-1992) were cultured in RPMI 1640+GlutaMAX (Thermo Fisher Scientific) containing 1% Pen/Strep and 10% FCS. Cell lines were authenticated by short tandem repeat profiling/karyotyping/isoenzyme analysis. All cells were passaged for a maximum of 2 months, after which new seed stocks were thawed for experimental use. In addition, all cell lines were routinely verified by growth rate, morphology, and/or flow cytometry and tested negative for mycoplasma using MycoAlert Mycoplasma Kit (Lonza, Breda, The Netherlands). Peripheral Blood Mononuclear Cells (PBMCs) were obtained from Sanquin Blood Bank (Amsterdam, the Netherlands) and isolated by Ficoll-Paque (GE Healthcare, Eindhoven, The Netherlands) from buffy coats. PBMCs were cultured using the previously described Rapid Expansion Protocol (REP; (31)) in RPMI containing 5% non-typed human serum (Sanquin Blood Bank), 1% Pen/Strep, and 50 μM β-Mercaptoethanol (collectively called HuRPMI).

Cloning of TCR Chains into Single Retroviral Vectors

The “minimally murinized” Vα16.1 and Vβ4.1 chains from an NY-ESO1₁₅₇₋₁₆₅/HLA*02 specific TCR, respectively named M2.2.3 and M1.KA,4.1, were generated as previously described (27). Additional partially murinized (regions or single residues) TCR chains were ordered from GeneArt (Thermo Fisher Scientific) or constructed via mutagenesis PCR. Cysteine modified chains were designed as reported previously (30). Variants of chimeric αβ/γδ TCRs were composed using the IMGT database (46). Sequences were codon optimized and ordered in an industrial resistance-gene harboring vector or as DNA strings (Geneart Life Technologies). DNA strings were processed using the TA TOPO cloning kit (Thermo Fisher Scientific) and cloned into the pCR™2.1-TOPO® vector, according to the manufacturer's protocol. All TCR chains were cloned separately into the retroviral vector pMP71 between the EcoRI and NotI restriction sites, using the indicated restriction enzymes and T4 DNA ligase (all from New England Biolabs, Ipswich Mass., United States). Transformation of ligated constructs was performed in JM109 competent E. Coli (Promega, Leiden, The Netherlands), and subsequent plasmid DNA isolation was conducted using Nucleobond® PC500, according to the manufacturer's protocol (Macherey-Nagel, Düren, Germany).

Retroviral Transduction of Primary T Cells and T Cell Lines

Phoenix-Ampho packaging cells were transfected using Fugene-HD (Promega) with env (pCOLT-GALV), gagpol (pHIT60), and separate pMP71 constructs containing α or β chains from an NY-ESO1₁₅₇₋₁₆₅/HLA-A*02 specific TCR (isolated from clone ThP2 (47)) kindly provided by Wolfgang Uckert (23), or containing TCRγ(G115)-T2A-TCRδ(G115)LM1 (20). PBMCs (preactivated with 50 IU/ml IL-2 (Proleukin, Novartis, Arnhem, The Netherlands) and 30 ng/ml anti-CD3 (clone OKT-3, Miltenyi Biotec, Bergisch Gladbach, Germany)), Jurma or Jurkat-76 cells were transduced twice within 48 hours with viral supernatant in 6-well plates (4×10\hat{ }6 cells/well) in the presence of 50 IU/ml IL-2 (PBMCs only) and 6 μg/ml polybrene (Sigma-Aldrich). After transduction, primary T cells were expanded by the addition of 50 μl/well anti-CD3/CD28 Dynabeads (Thermo Fisher Scientific) and 50 IU/ml IL-2.

Purification of Engineered T Cells by MACS Depletion of Poorly and Non-Engineered Immune Cells

Transduced primary T cells were incubated with biotin-labeled anti-human αβTCR antibody (clone BW242/412; Miltenyi Biotec), followed by incubation with an anti-biotin antibody coupled to magnetic beads (anti-biotin MicroBeads; Miltenyi Biotec) (20). Next, the cell suspension was applied to an LD column in a QuadroMACS™ Separator. αβTCR-positive T cells were depleted by MACS cell separation according to the manufacturer's protocol (Miltenyi Biotec).

In Silico TCR Modelling

The structure of different murinized constant domains was predicted using SWISS-MODEL (48) on the modeled template of the β chain of the human JKF6 T-cell receptor (PDB entry code: 4ZDH). The structure of the murinized constant domains when binding H57-597 was modeled on the template of the β chain of the murine N15 T-cell receptor (PDB entry code: 1NFD) (49). Structure visualizations were performed using PyMol Molecular Graphics System (50).

Chimeric Antibody Production and Purification

Hamster-human (IgG1) chimeric H57-597 antibody was generated using Lonza expression vectors (pEE14·4-kappaLC, pEE14·4-IgG1) (51, 52). The antibody was produced by transient transfection of HEK293F cells with the heavy chain coding plasmid, the light chain coding plasmid and pAdVAntage (Accession Number U47294; Promega), using 293fectin transfection reagent (Invitrogen) following the manufacturer's instructions. Antibody-containing supernatant was harvested 4 days after transfection and purified by affinity chromatography using HiTrap Protein G HP antibody purification columns (GE Healthcare).

Sequencing

DNA sequences of cloning intermediates and final constructs in pMP71 were verified by Barcode Sequencing (Baseclear, Leiden, The Netherlands). 75 μg plasmid DNA and 25 pmol primer specific for the pCR™2.1-TOPO® vector or pMP71 vector were premixed in a total of 20 μl and sent to Baseclear for Sanger sequencing.

Flow Cytometry

Cells were stained with Vβ4-FITC (TRBV29-1, clone WJF24; Beckman Coulter), αβTCR-PE (clone BW242/412; Miltenyi Biotec), CD3-PB (clone UCHT1; BD), CD4-PeCy7 (clone RPA-T4; eBioscience, Thermo Fisher Scientific), CD8-APC (clone RPA-T8; BD), CD8-PB (clone SK1; Biolegend), or RPE-conjugated NY-ESO-1₁₅₇₋₁₆₅ HLA*02:01 (SLLMWITQV) pentamer (ProImmune, Oxford, United Kingdom). Samples were fixed using 1% PFA in PBS, measured on a FACSCanto-II flow cytometer (BD), and analyzed using FACSDiva (BD) or FlowJo (Three Star Inc.) software.

ELISA

Effector and target cells (E:T 50,000:50,000) were incubated for 16 hours after which supernatant was harvested. IFNγ ELISA was performed using ELISA-ready-go! Kit (eBioscience) following the manufacturer's instructions.

MMAE ADC Construction

Chimeric H57-MC-VC-PAB-MMAE was constructed using a kit from CellMosaic, (Woburn, Mass., United States) following the manufacturer's instructions.

### REFERENCES

1. Chabannon C, Kuball J, Bondanza A, Dazzi F, Pedrazzoli P, Toubert A, et al. Hematopoietic stem cell transplantation in its 60s: A platform for cellular therapies. Sci Transl Med. 2018; 10(436).

2. de Witte M A, Kierkels G J, Straetemans T, Britten C M, Kuball J. Orchestrating an immune response against cancer with engineered immune cells expressing alphabetaTCRs, CARs, and innate immune receptors: an immunological and regulatory challenge. Cancer Immunol Immunother. 2015; 64(7):893-902.

3. Morgan R A, Chinnasamy N, Abate-Daga D, Gros A, Robbins P F, Zheng Z, et al. Cancer regression and neurological toxicity following anti-MAGE-A3 TCR gene therapy. J Immunother. 2013; 36(2):133-51.

4. Abate-Daga D, Hanada K, Davis J L, Yang J C, Rosenberg S A, Morgan R A. Expression profiling of TCR-engineered T cells demonstrates overexpression of multiple inhibitory receptors in persisting lymphocytes. Blood. 2013; 122(8):1399-410.

5. Gnjatic S, Nishikawa H, Jungbluth A A, Gure A O, Ritter G, Jager E, et al. NY-ESO-1: review of an immunogenic tumor antigen. Adv Cancer Res. 2006; 95:1-30.

6. Orlando D, Miele E, De Angelis B, Guercio M, Boffa I, Sinibaldi M, et al. Adoptive immunotherapy using PRAME-specific T cells in medulloblastoma. Cancer Res. 2018.

7. Rapoport A P, Stadtmauer E A, Binder-Scholl G K, Goloubeva O, Vogl D T, Lacey S F, et al. NY-ESO-1-specific TCR-engineered T cells mediate sustained antigen-specific antitumor effects in myeloma. Nat Med. 2015; 21(8):914-21.

8. de Witte M A, Jorritsma A, Kaiser A, van den Boom M D, Dokter M, Bendle G M, et al. Requirements for effective antitumor responses of TCR transduced T cells. J Immunol. 2008; 181(7):5128-36.

9. Abad J D, Wrzensinski C, Overwijk W, De Witte M A, Jorritsma A, Hsu C, et al. T-cell receptor gene therapy of established tumors in a murine melanoma model. J Immunother. 2008; 31(1):1-6.

10. Fehse B, Richters A, Putimtseva-Scharf K, Klump H, Li Z, Ostertag W, et al. CD34 splice variant: an attractive marker for selection of gene-modified cells. Mol Ther. 2000; 1(5 Pt 1):448-56.

11. Orchard P J, Blazar B R, Burger S, Levine B, Basso L, Nelson D M, et al. Clinical-scale selection of anti-CD3/CD28-activated T cells after transduction with a retroviral vector expressing herpes simplex virus thymidine kinase and truncated nerve growth factor receptor. Hum Gene Ther. 2002; 13(8):979-88.

12. Lamers C H, Willemsen R, van E P, van Steenbergen-Langeveld S, Broertjes M, Oosterwijk-Wakka J, et al. Immune responses to transgene and retroviral vector in patients treated with ex vivo-engineered T cells. Blood. 2011; 117(1):72-82.

13. Neelapu S S, Tummala S, Kebriaei P, Wierda W, Gutierrez C, Locke F L, et al. Chimeric antigen receptor T-cell therapy—assessment and management of toxicities. Nat Rev Clin Oncol. 2018; 15(1):47-62.

14. Linette G P, Stadtmauer E A, Maus M V, Rapoport A P, Levine B L, Emery L, et al. Cardiovascular toxicity and titin cross-reactivity of affinity-enhanced T cells in myeloma and melanoma. Blood. 2013; 122(6):863-71.

15. Traversari C, Marktel S, Magnani Z, Mangia P, Russo V, Ciceri F, et al. The potential immunogenicity of the TK suicide gene does not prevent full clinical benefit associated with the use of TK-transduced donor lymphocytes in HSCT for hematologic malignancies. Blood. 2007; 109(11):4708-15.

16. Traversari C, Marktel S, Magnani Z, Mangia P, Russo V, Ciceri F, et al. The potential immunogenicity of the TK suicide gene does not prevent full clinical benefit associated with the use of TK-transduced donor lymphocytes in HSCT for hematologic malignancies. Blood. 2007; 109(11):4708-15.

17. Kieback E, Charo J, Sommermeyer D, Blankenstein T, Uckert W. A safeguard eliminates T cell receptor gene-modified autoreactive T cells after adoptive transfer. Proc Natl Acad Sci USA. 2008; 105(2):623-8.

18. Kuball J, Hauptrock B, Malina V, Antunes E, Voss R H, Wolfl M, et al. Increasing functional avidity of TCR-redirected T cells by removing defined N-glycosylation sites in the TCR constant domain. JExpMed. 2009; 206(2):463-75.

19. Introna M, Barbui A M, Bambacioni F, Casati C, Gaipa G, Borleri G, et al. Genetic modification of human T cells with CD20: a strategy to purify and lyse transduced cells with anti-CD20 antibodies. Hum Gene Ther. 2000; 11(4):611-20.

20. Straetemans T, Grunder C, Heijhuurs S, Hol S, Slaper-Cortenbach I, Bonig H, et al. Untouched GMP-Ready Purified Engineered Immune Cells to Treat Cancer. Clinical cancer research: an official journal of the American Association for Cancer Research. 2015; 21(17):3957-68.

21. Straetemans T, Kierkels G J J, Doorn R, Jansen K, Heijhuurs S, Dos Santos J M, et al. GMP-Grade Manufacturing of T Cells Engineered to Express a Defined gammadeltaTCR. Front Immunol. 2018; 9:1062.

22. Schumm M, Lang P, Bethge W, Faul C, Feuchtinger T, Pfeiffer M, et al. Depletion of T-cell receptor alpha/beta and CD19 positive cells from apheresis products with the CliniMACS device. Cytotherapy. 2013; 15(10):1253-8.

23. Sommermeyer D, Uckert W. Minimal amino acid exchange in human TCR constant regions fosters improved function of TCR gene-modified T cells. J Immunol. 2010; 184(11):6223-31.

24. Stanislawski T, Voss R H, Lotz C, Sadovnikova E, Willemsen R A, Kuball J, et al. Circumventing tolerance to a human MDM2-derived tumor antigen by TCR gene transfer. Nat Immunol. 2001; 2(10):962-70.

25. Kuball J, Schmitz F W, Voss R H, Ferreira E A, Engel R, Guillaume P, et al. Cooperation of human tumor-reactive CD4+ and CD8+ T cells after redirection of their specificity by a high-affinity p53A2.1-specific TCR. Immunity. 2005; 22(1):117-29.

26. Davis J L, Theoret M R, Zheng Z, Lamers C H, Rosenberg S A, Morgan R A. Development of human anti-murine T-cell receptor antibodies in both responding and nonresponding patients enrolled in TCR gene therapy trials. Clin Cancer Res. 2010; 16(23):5852-61.

27. Sommermeyer D, Uckert W. Minimal amino acid exchange in human TCR constant regions fosters improved function of TCR gene-modified T cells. J Immunol. 2010; 184(11):6223-31.

28. Voss R H, Kuball J, Theobald M. Designing TCR for Cancer Immunotherapy. Methods MolMed. 2005; 109:229-56.

29. Voss R H, Kuball J, Engel R, Guillaume P, Romero P, Huber C, et al. Redirection of T cells by delivering a transgenic mouse-derived MDM2 tumor antigen-specific TCR and its humanized derivative is governed by the CD8 coreceptor and affects natural human TCR expression. ImmunolRes. 2006; 34(1):67-87.

30. Kuball J, Dossett M L, Wolfl M, Ho W Y, Voss R H, Fowler C, et al. Facilitating matched pairing and expression of TCR chains introduced into human T cells. Blood. 2007; 109(6):2331-8.

31. Marcu-Malina V, Heijhuurs S, van Buuren M, Hartkamp L, Strand S, Sebestyen Z, et al. Redirecting alphabeta T cells against cancer cells by transfer of a broadly tumor-reactive gammadeltaT-cell receptor. Blood. 2011; 118(1):50-9.

32. Admiraal R, Nierkens S, de Witte M A, Petersen E J, Fleurke G J, Verrest L, et al. Association between anti-thymocyte globulin exposure and survival outcomes in adult unrelated haemopoietic cell transplantation: a multicentre, retrospective, pharmacodynamic cohort analysis. Lancet Haematol. 2017; 4(4):e183-e91.

33. Caculitan N G, Dela Cruz Chuh J, Ma Y, Zhang D, Kozak K R, Liu Y, et al. Cathepsin B Is Dispensable for Cellular Processing of Cathepsin B-Cleavable Antibody-Drug Conjugates. Cancer Res. 2017; 77(24):7027-37.

34. Gebleux R, Stringhini M, Casanova R, Soltermann A, Neri D. Non-internalizing antibody-drug conjugates display potent anti-cancer activity upon proteolytic release of monomethyl auristatin E in the subendothelial extracellular matrix. Int J Cancer. 2017; 140(7):1670-9.

35. Locatelli F, Merli P, Pagliara D, Li Pira G, Falco M, Pende D, et al. Outcome of children with acute leukemia given HLA-haploidentical HSCT after alphabeta T-cell and B-cell depletion. Blood. 2017; 130(5):677-85.

36. Provasi E, Genovese P, Lombardo A, Magnani Z, Liu P Q, Reik A, et al. Editing T cell specificity towards leukemia by zinc finger nucleases and lentiviral gene transfer. NatMed. 2012; 18(5):807-15.

37. Gaj T, Gersbach C A, Barbas C F, III. ZFN, TALEN, and CRISPR/Cas-based methods for genome engineering. Trends Biotechnol. 2013.

38. Kuball J, Dossett M L, Wolfl M, Ho W Y, Voss R H, Fowler C, et al. Facilitating matched pairing and expression of TCR chains introduced into human T cells. Blood. 2007; 109(6):2331-8.

39. Lock D, Mockel-Tenbrinck N, Drechsel K, Barth C, Mauer D, Schaser T, et al. Automated Manufacturing of Potent CD20-Directed Chimeric Antigen Receptor T Cells for Clinical Use. Hum Gene Ther. 2017; 28(10):914-25.

40. Hodi F S, O'Day S J, McDermott D F, Weber R W, Sosman J A, Haanen J B, et al. Improved survival with ipilimumab in patients with metastatic melanoma. N Engl J Med. 2010; 363(8):711-23.

41. Smith M, Zakrzewski J, James S, Sadelain M. Posttransplant chimeric antigen receptor therapy. Blood. 2018.

42. Schmitt T M, Stromnes I M, Chapuis A G, Greenberg P D. New Strategies in Engineering T-cell Receptor Gene-Modified T cells to More Effectively Target Malignancies. Clinical cancer research: an official journal of the American Association for Cancer Research. 2015; 21(23):5191-7.

43. Bijen H M, van der Steen D M, Hagedoorn R S, Wouters A K, Wooldridge L, Falkenburg J H F, et al. Preclinical Strategies to Identify Off-Target Toxicity of High-Affinity TCRs. Mol Ther. 2018.

44. Di Stasi A, Tey S K, Dotti G, Fujita Y, Kennedy-Nasser A, Martinez C, et al. Inducible apoptosis as a safety switch for adoptive cell therapy. N Engl J Med. 2011; 365(18): 1673-83.

45. Aarnoudse C A, Kruse M, Konopitzky R, Brouwenstijn N, Schrier P I. TCR reconstitution in Jurkat reporter cells facilitates the identification of novel tumor antigens by cDNA expression cloning. Int J Cancer. 2002; 99(1):7-13.

46. IMGT®, the international ImMunoGeneTics information system®. www.imgt.org.

47. Kronig H, Hofer K, Conrad H, Guilaume P, Muller J, Schiemann M, et al. Allorestricted T lymphocytes with a high avidity T-cell receptor towards NY-ESO-1 have potent anti-tumor activity. Int J Cancer. 2009; 125(3):649-55.

48. Bertoni M, Kiefer F, Biasini M, Bordoli L, Schwede T. Modeling protein quaternary structure of homo- and hetero-oligomers beyond binary interactions by homology. Sci Rep. 2017; 7(1):10480.

49. Wang J, Lim K, Smolyar A, Teng M, Liu J, Tse A G, et al. Atomic structure of an alphabeta T cell receptor (TCR) heterodimer in complex with an anti-TCR fab fragment derived from a mitogenic antibody. EMBO J. 1998; 17(1):10-26.

50. Schrodinger, L L C. The PyMOL Molecular Graphics System, Version 1.8. 2015.

51. Boross P, Lohse S, Nederend M, Jansen J H, van Tetering G, Dechant M, et al. IgA EGFR antibodies mediate tumour killing in vivo. EMBO Mol Med. 2013; 5(8):1213-26.

52. Meyer S, Evers M, Jansen J H M, Buijs J, Broek B, Reitsma S E, et al. New insights in Type I and II CD20 antibody mechanisms-of-action with a panel of novel CD20 antibodies. Br J Haematol. 2018; 180(6):808-20.

### EXAMPLE 2

Construct Design

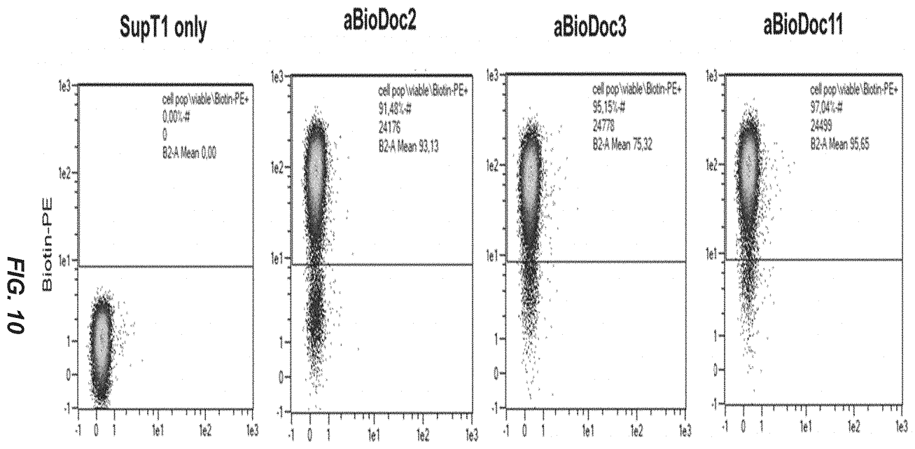

In order to develop Biotin-specific Adapter-TCRs (Adapter TCR) recognizing biotin as well as parts of the linker moiety connecting biotin to an antibody recognizing the target cell epitope CD20, a scFv (single chain variable fragment) was designed based on the variable heavy (VH) and variable light (VL) chains of an anti-biotin antibody in VH-VL orientation (SEQ ID NO:37). This scFv was used to replace the variable domains of the alpha and beta T cell receptor (TCR) chain, respectively. Thus, Biotin-specific Adapter-TCRs consist of T cell receptor alpha constant (TRAC) (Uniprot ID: P01848) and T cell receptor beta constant 1 or 2 (Uniprot ID: P01850 or A0A5B9) or modifications thereof including additional Cysteines in both chains as described by Kuball et al. (Blood. 2007 Mar. 15; 109(6):2331-2338), the exchange of TRAC transmembrane domain (TM) against the TM derived from T cell receptor delta constant (TRDC) (Uniprot ID: B7Z8K6) and/or the exchange of the beta constant TM against the TM derived from T cell receptor gamma constant 1 (TRGC1) (Uniprot ID: P0CF51) or the exchange of glutamic acid against lysine at position 18 in the beta chain as described by Sommermeyer et al. (J Immunol 2010; 184:6223-6231). In addition, some constructs contained either the 2/11 or the 9/11 mutation. All Uniprot-derived sequences used have a sequence identity of at least 70% to the original sequence. A graphical representation of different constructs tested is given in FIG. 9.

Production of Lentiviral Particles

Lentiviral particles were produced using standard protocols known by the skilled person. After production, LV particles were resuspended in TexMACS medium (100× concentration) and directly frozen at −80° C.

Determination of Lentiviral Titer Using SupT1 Cells

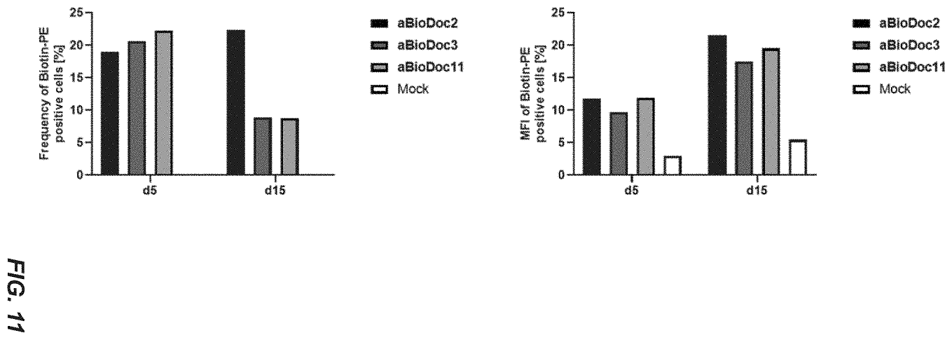

3E5 SupT1 cells were transduced with 10 μl of LV particles encoding the Adapter TCRs (FIG. 10). The transduction efficiency was measured 3 days post transduction by flow analysis using the viability dye 7-AAD and Biotin-PE (Miltenyi Biotec) allowing to calculate the titer.

Assessing the Functionality of Adapter TCRs In Vitro

Transduction of T Cells:

PBMCs were isolated from blood samples from healthy donors by centrifugation on Pancoll (PANBiotech). Subsequently, PAN T cells were isolated using the PAN T cell isolation Kit (Miltenyi Biotec). For the activation and expansion, T cells were seeded in 24-well plates at a concentration of 1E6 cells/mL in 2 ml of TexMACS medium containing TransAct supplemented with IL-7 (12.5 ng/mL) and IL-15 (12.5 ng/mL) (Miltenyi Biotec). 2E6 activated T cells were transduced with an MOI of ˜4 on day 1 after activation. On day 5, T cells were stained with the viability dye 7-AAD and Biotin-PE (Miltenyi Biotec) to measure the transduction efficacy by flow analysis. T cells were then continuously expanded in TexMACS medium with IL-7 (12.5 ng/mL) and IL-15 (12.5 ng/m) (Miltenyi Biotec). The Adapter TCR-expression was determined again on day 15 (FIG. 11).

Flow-Based Killing Assay:

On day 15, two different mono-biotinylated adapters namely Rituxifab (a CD20-specific Fragment antigen binding (fab)) and Rituximab (a CD20-specific monoclonal antibody (mab)) were used to assess the functionality of Biotin-specific Adapter TCRs using a flow-based killing assay. Therefore GFP-expressing Raji cells were used as target cell line. For the co-culture, Adapter TCR-positive T cells or Mock T cells (negative control) were co-cultured with GFP-expressing Raji cells with or without the addition of Rituximab or Rituxifab. For this, 5E5 target cells and 5E5 transduced effector cells (E:T ratio of 1:1) were co-cultured in a 96-well round bottom plate in TexMACS medium with Rituximab (1 μg/ml) or Rituxifab (1 μg/ml) in respective wells for 18 h at 37° C. and 5% CO₂. In addition, Adapter TCR-expressing T cells were co-cultured with biotinylated GFP positive Rajis (Bio-Raji) as described above. For biotinylation, GFP-expressing Rajis were incubated with NHS-LCLC-Biotin (20 ng/μl) in PBS, washed twice with PBS-glycine and cultivated in RPMI+2 mM L-glutamine+10% FCS.

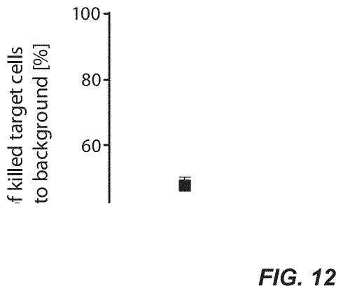

Finally, co-cultures were analyzed using flow cytometry, in which, after exclusion of dead cells, GFP-expression inversely correlated with target cell death as well-known in the art (FIG. 12).

Intracellular Cytokine Production Assay:

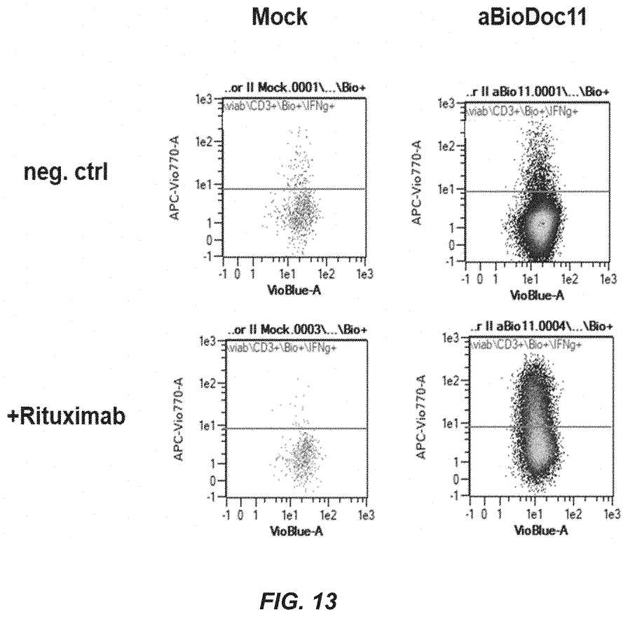

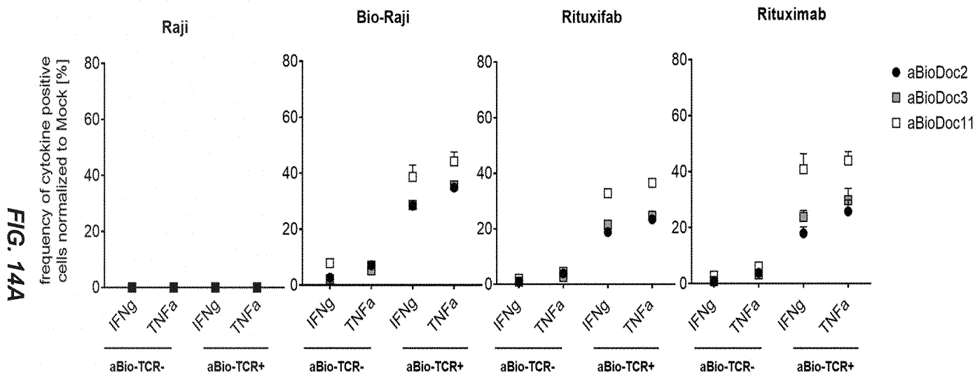

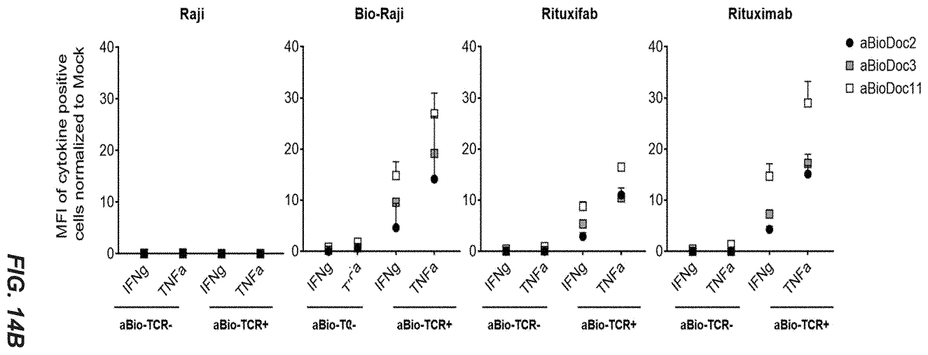

To analyze the cytokine production of Adapter TCR-positive T cells, a co-culture of 2.5E5 effector cells and 5E4 target cells (E:T 0.25:1) was set-up as described above. Adapter TCR-expressing T cells or Mock T cells (negative control) were co-cultured with Bio-Rajis or with Rajis in the absence or presence of Rituximab or Rituxifab (1 μg/ml, respectively). In addition, Brefeldin A (1 μg/ml) was added to each well to disrupt cytokine release. After 4 hours of co-culture at 37° C. and 5% CO₂, intracellular cytokine staining was performed using Inside fix, Inside Perm, Viability dye-Viogreen, CD3-VioBlue, Biotin-PE, TNFa-APC and IFNg-APC-Vio770 (Miltenyi Biotec) according to the manufacturer's instructions. Intracellular cytokine expression was measured by flow analysis as representatively shown in FIG. 13. FIG. 14 summarizes both, frequencies of IFNg- and TNFa-positive cells (FIG. 14A) as well as MFI (FIG. 14B) of the respective populations.

Discussion/Conclusion

The potential of genetically engineered T cells for the treatment of advanced hematopoietic malignancies, solid cancer as well as infectious diseases has been demonstrated in several clinical trials. Especially CAR T cells have shown remarkable success in several clinical studies against B cell malignancies. Here, however, a novel technology has been developed in which the variable domain of both TCR chains was replaced against Biotin-specific scFv's allowing to redirect genetically engineered T cells using biotinylated binders. Consequently, this Adapter approach combines the potential of CARs, the flexibility of an adapter approach and the endogenous and powerful T cell activating cascade. Therefore, three different Adapter TCR versions namely aBioDoc2, aBioDoc3 and aBioDoc11 were cloned, lentiviral particles produced and used to genetically engineer both SupT1 cells as well as T cells. After confirming the transgenic expression of all versions, the cytolytic potential of these T cells was assessed in vitro. Finally, the data confirmed the possibility to specifically kill either biotinylated target cells or cells labeled with biotinylated binders for instance Rituximab or Rituxifab. Mock cells co-cultured with (biotinylated) target cells, although in the presence of Rituximab or Rituxifab, did not show a comparable cytolytic potential. This data was further confirmed by intracellular cytokine stainings performed after 4 h co-culture assays in which a specific release of IFNg and TNFa could be shown for aBio-TCR+ T cells only in the presence of biotinylated cells or when using biotinylated binders. In conclusion, the novel Adapter TCR concept specifically activates T cells only in the presence of biotinylated target cells (directly biotinylated or labeled with biotinylated binders) resulting in a respective tumor cell lysis as shown for all Adapter TCR versions tested aBioDoc2, aBioDoc3 and aBioDoc11.

1. An immune cell expressing an immune receptor, the immune cell comprising:
i) a polypeptide having
an antigen binding domain;
a TCR α-chain constant domain; and
a TCR δ-chain transmembrane domain or γ-chain transmembrane domain; and/or
ii) a polypeptide having
an antigen binding domain;
a TCR β-chain constant domain; and
a TCR γ-chain transmembrane domain or δ-chain transmembrane domain.

2. The immune cell of claim 1, wherein the polypeptide under i) and the polypeptide under ii) are linked by a Cysteine bridge.

3. The immune cell of claim 1, wherein
the antigen binding domain under i) is an epitope binding peptide, preferably an scFv, V-Nar, or VhH; and/or
the antigen binding domain under ii) is an epitope binding peptide, preferably an scFv, V-Nar or VhH.

4. The immune cell of claim 1,
wherein the TCR α-chain constant domain is replaced by a TCR δ-chain constant domain or TCR γ-chain constant domain and/or
wherein the TCR β-chain constant domain is replaced by a TCR δ-chain constant domain or γ-chain constant domain.

5. The immune cell of claim 1, wherein the antigen binding domain under i) and/or the antigen binding domain under ii) comprises
an amino acid sequence having at least 70% sequence identity with SEQ ID NO:1; and/or
an amino acid sequence having at least 70% sequence identity with SEQ ID NO:2.

6. The immune cell of claim 1, wherein
the TCR α-chain constant domain comprises an amino acid sequence having at least 70% sequence identity with SEQ ID NO:3;
the TCR δ-chain transmembrane domain comprises an amino acid sequence having at least 70% sequence identity with SEQ ID NO:4;
the TCR β-chain constant domain comprises an amino acid sequence having at least 70% sequence identity with SEQ ID NO:5; and/or
the TCR γ-chain transmembrane domain comprises an amino acid sequence having at least 70% sequence identity with SEQ ID NO:6.

7. The immune cell of claim 1, wherein the polypeptide under i) and/or the polypeptide under ii) further has a cytoplasmic signaling domain.

8. The immune cell of claim 1, wherein the immune cell expressing the immune receptor is in combination with a tagged polypeptide, and wherein the antigen binding domain under i) of the immune receptor and/or the antigen binding domain under ii) of the immune receptor is specific for a tag of the tagged polypeptide.

9. The immune cell of claim 8, wherein the tag comprises an amino acid sequence having at least 70% sequence identity with SEQ ID NO:36, characterized in that the amino acid sequence comprises:
a Histidine or conservative substitution thereof at a position corresponding to position 88 as shown in SEQ ID NO:10;
a Histidine or conservative substitution thereof at a position corresponding to position 101 as shown in SEQ ID NO:10.
a Lysine or conservative substitution thereof at a position corresponding to position 108 as shown in SEQ ID NO:10;
an amino acid other than Threonine at a position corresponding to position 110 as shown in SEQ ID NO: 9N0:9 and/or SEQ ID NO:10;
a Glutamic acid or conservative substitution thereof at a position corresponding to position 111 as shown in SEQ ID NO:10;
an amino acid other than Aspartic acid at a position corresponding to position 112 as shown in SEQ ID NO: 9N0:9 and/or SEQ ID NO:10.
a Serine or conservative substitution thereof at a position corresponding to position 113 as shown in SEQ ID NO:10;
an Asparagine or conservative substitution thereof at a position corresponding to position 120 as shown in SEQ ID NO:10; and/or
an Isoleucine or conservative substitution thereof at a position corresponding to position 121 as shown in SEQ ID NO:10.

10. The immune cell of claim 1, wherein the polypeptide under i), the polypeptide under ii), and/or the tag according to claim 8 comprises an amino acid sequence having at least 70% sequence identity with SEQ ID NO:36, characterized in that the amino acid sequence comprises:
an Histidine or conservative substitution thereof at a position corresponding to position 88 as shown in SEQ ID NO:10;
an Histidine or conservative substitution thereof at a position corresponding to position 101 as shown in SEQ ID NO:10.
a Lysine or conservative substitution thereof at a position corresponding to position 108 as shown in SEQ ID NO:10;
an amino acid other than Threonine at a position corresponding to position 110 as shown in SEQ ID NO:9 and/or SEQ ID NO:10;
a Glutamic acid or conservative substitution thereof at a position corresponding to position 111 as shown in SEQ ID NO:10;
an amino acid other than Aspartic acid at a position corresponding to position 112 as shown in SEQ ID NO:9 and/or SEQ ID NO:10.
a Serine or conservative substitution thereof at a position corresponding to position 113 as shown in SEQ ID NO:10;
an Asparagine or conservative substitution thereof at a position corresponding to position 120 as shown in SEQ ID NO:10; and/or
an Isoleucine or conservative substitution thereof at a position corresponding to position 121 as shown in SEQ ID NO:10.

11. The immune cell of claim 9, wherein
the amino acid other than Threonine at a position corresponding to position 110 is Proline or conservative substitution thereof; and/or
the amino acid other than Aspartic acid at a position corresponding to position 112 is Glycine or conservative substitution thereof.

12. The immune cell of claim 9, wherein the amino acid sequence having at least 70% sequence identity with SEQ ID NO:36 further comprises:
an Asparagine or conservative substitution at a position corresponding to position 106 as shown in SEQ ID NO:10; and/or
an Alanine or conservative substitution at a position corresponding to position 114 as shown in SEQ ID NO:10.

13. The immune cell of claim 1, wherein the immune cell is selected from the group consisting of an human immune cell, a human T cell, human NK cell, and a human NK T cell.

14. A method of using the immune cell or immune cells of claim 1, wherein the method comprises
administering the immune cell(s) expressing to a subject in need thereof.

15. The method according to claim 14, wherein the subject has cancer.

16. The method according to claim 14, wherein the immune cell(s) are administered separately.

17. The immune cell of claim 3, wherein the antigen binding domain under i) and the antigen binding domain of ii) are the same.

18. The immune cell of claim 3, wherein the antigen binding domain under i) and the antigen binding domain of ii) are different.

19. The immune cell of claim 7, wherein the polypeptide under i) and/or the polypeptide under ii) has a CD3 signaling domain.

20. The immune cell of claim 8, wherein the tagged polypeptide can specifically bind an antigen on a target cell.

21. The immune cell of claim 9, wherein the tagged polypeptide comprises an antibody or antigen binding fragment thereof.

22. The immune cell of claim 10, wherein
the amino acid other than Threonine at a position corresponding to position 110 is Proline or a conservative substitution thereof; and/or
the amino acid other than Aspartic acid at a position corresponding to position 112 is Glycine or a conservative substitution thereof.


SEQUENCE LISTING (37 sequences)

SEQ ID NO: 1 - protein, 107 aa, Artificial Sequence
  Light chain variable region of humanized anti-MuTCR-beta antibody
YELIQPSSASVTVGETVKITCSGDQLPKNFAYWFQQKSDKNILLLIYMDNKRPSGIPERFSGSTSGTTATLTISGAQPEDEAAYYCLSSYGDNNDLVFGSGTQLTVL

SEQ ID NO: 2 - protein, 120 aa, Artificial Sequence
  Heavy chain variable region of humanized anti-MuTCR-beta antibody
EVYLVESGGDLVQPGSSLKVSCAASGFTFSDFWMYWVRQAPGKGLEWVGRIKNKPNNYATEYADSVRGRFTISRDDSRNSIYLQMNRLRVDDTAIYYCTRAGRFDHFDYWGQGTMVTVSS

SEQ ID NO: 3 - protein, 115 aa, Artificial Sequence
  TCR alpha-chain constant domain
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLS

SEQ ID NO: 4 - protein, 23 aa, Artificial Sequence
  TCR delta-chain transmembrane domain
LGLRMLFAKTVAVNFLLTAKLFF

SEQ ID NO: 5 - protein, 179 aa, Artificial Sequence
  TCR beta-chain constant domain
EDLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 6 - protein, 23 aa, Homo sapiens
YYMYLLLLLKSVVYFAIITCCLL

SEQ ID NO: 7 - protein, 34 aa, Homo sapiens
QNPRNHFRCQVQFYGLSENDEWTQDRAKPVTQIV

SEQ ID NO: 8 - protein, 34 aa, Artificial Sequence
  Murine TCR-beta chain Domain 3
HNPRNHFRCQVQFHGLSEEDKWPEGSPKPVTQNI

SEQ ID NO: 9 - protein, 179 aa, Artificial Sequence
  Human TCR-beta chain comprising Domain 3 with 2 murinized AA residues
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWPQGRAKPVTQIVSAEAWGRADCGFTSESYQQGVLSATILYEILLGKATLYAVLVSALVLMAMVKRKDSRG

SEQ ID NO: 10 - protein, 179 aa, Artificial Sequence
  Human TCR-beta chain comprising Domain 3 with 9 murinized AA residues
EDLKNVFPPKVAVFEPSEAEISHTQKATLVCLATGFYPDHVELSWWVNGKEVHSGVSTDPQPLKEQPALNDSRYCLSSRLRVSATFWHNPRNHFRCQVQFHGLSENDKWPEGSAKPVTQNISAEAWGRADCGFTSESYQQGVLSATILYEILLGKATLYAVLVSALVLMAMVKRKDSRG

SEQ ID NO: 11 - protein, 486 aa, Artificial Sequence
  Complete CAR sequence
MALPVTALLLPLALLLHAARPYELIQPSSASVTVGETVKITCSGDQLPKNFAYWFQQKSDKNILLLIYMDNKRPSGIPERFSGSTSGTTATLTISGAQPEDEAAYYCLSSYGDNNDLVFGSGTQLTVLGGGGSGGGGSGGGGSEVYLVESGGDLVQPGSSLKVSCAASGFTFSDFWMYWVRQAPGKGLEWVGRIKNKPNNYATEYADSVRGRFTISRDDSRNSIYLQMNRLRVDDTAIYYCTRAGRFDHFDYWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 272 aa, Homo sapiens
MEKNPLAAPLLILWTHLDCVSILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 312 aa, Homo sapiens
MGIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - protein, 315 aa, Homo sapiens
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 15 - protein, 292 aa, Homo sapiens
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 16 - protein, 306 aa, Mus musculus
MATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 17 - protein, 306 aa, Mus musculus
MNKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGDWGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 18 - protein, 269 aa, Mus musculus
MVLALLPVLGIHFVLRDAQAQSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 19 - protein, 268 aa, Mus musculus
MKRLLCSLLGLLCTQVCWVKGQQVQQSPASLVLQEGENAELQCNFSSTATRLQWFYQRPGGSLVSLLYNPSGTKHTGRLTSTTVTKERRSSLHISSSQTTDSGTYFCATSSVNTGNYKYVFGAGTRLKVIAHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 20 - protein, 141 aa, Homo sapiens
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - protein, 72 aa, Homo sapiens
IQNPEPAVYQLKDPRSQDSTLCLFTDFDSQINVPKTMESGTFITDKTVLDMKAMDSKSNGAIAWSNQTSFTC

SEQ ID NO: 22 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 23 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 24 - protein, 172 aa, Homo sapiens
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 172 aa, Homo sapiens
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 26 - protein, 177 aa, Homo sapiens
PSYTGGYADKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 27 - protein, 152 aa, Homo sapiens
SQPPAKPSVFIMKNGTNVACLVKDFYPKEVTISLRSSKKIVEFDPAIVISPSGKYSAVKLGQYGDSNSVTCSVQHNSETVHSTDFEPYANSFNNEKLPEPENDTQISEPCYGPRVTVHTEKVNMMSLTVLGLRLLFAKTIAINFLLTVKLFF

SEQ ID NO: 28 - protein, 172 aa, Homo sapiens
KQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 29 - protein, 109 aa, Homo sapiens
KQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKT

SEQ ID NO: 30 - protein, 204 aa, Homo sapiens
KQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVTTVDPKYNYSKDANDVITMDPKDNWSKDANDTLLLQLTNTSAYYMYLLLLLKSVVYFAIITCCLLGRTAFCCNGEKS

SEQ ID NO: 31 - protein, 171 aa, Homo sapiens
KRLDADISPKPTIFLPSVAETNLHKTGTYLCLLEKFFPDVIRVYWKEKDGNTILDSQEGDTLKTNDTYMKFSWLTVPERAMGKEHRCIVKHENNKGGADQEIFFPSIKKVAVSTKPTTCWQDKNDVLQLQFTITSAYYTYLLLLLKSVIYLAIISFSLLRRTSVCGNEKKS

SEQ ID NO: 32 - protein, 166 aa, Homo sapiens
KRLDADISPKPTIFLPSVAETNLHKTGTYLCLLEKFFPDVIRVYWKEKNGNTILDSQEGDTLKTKGTYMKFSWLTVPERAMGKEHSCIVKHENNKGGADQEIFFPSIKKVATTCWQDKNDVLQFQFTSTSAYYTYLLLLLKSVIYLAIISFSLLRRTSVCGNEKKS

SEQ ID NO: 33 - protein, 171 aa, Homo sapiens
KKLDADISPKPTIFLPSVAETNLHKTGTYLCVLEKFFPDVIRVYWKEKKGNTILDSQEGDMLKTNDTYMKFSWLTVPERSMGKEHRCIVKHENNKGGADQEIFFPTIKKVAVSTKPTTCWQDKNDVLQLQFTITSAYYTYLLLLLKSVIYLAIISFSLLRRTSVCCNEKKS

SEQ ID NO: 34 - protein, 166 aa, Homo sapiens; identical to SEQ ID NO: 32

SEQ ID NO: 35 - protein, 34 aa, Artificial Sequence
  Human TCR-beta chain Domain 3 with 2 murinized amino acid residues
QNPRNHFRCQVQFYGLSENDEWPQGRAKPVTQIV

SEQ ID NO: 36 - protein, 34 aa, Artificial Sequence
  Human TCR-beta chain Domain 3 with 9 murinized amino acid residues
HNPRNHFRCQVQFHGLSENDKWPEGSAKPVTQNI

SEQ ID NO: 37 - protein, 249 aa, Artificial Sequence
  Single chain variable fragment derived from anti-biotin antibody
EVQLVESGGGLVQPGGSLRLSCAASGFTFSNYAMSWVRQAPGKGLEWVATITGGGSYTYYPDSVKGRFTISRDNSKNTLYLQMNSLRAEDTAVYYCVRQRVGDYVSSLLGYWGQGTLVTVSSGGGGSGGGGSGGGGSDVVMTQSPLSLPVTLGQPASISCRSSQSLVHSNGNTYLHWFLQRPGQSPRLLIYKVSNRFSGVPDRFSGSGSGTDFTLKISRVEAEDVGVYYCSQSTHVPYTFGQGTKLEIK